(19) United States
(12) Patent Application Publication
(10) Pub. No.: US 2025/0242036 A1
(43) Pub. Date: Jul. 31, 2025

(54) TRANS-MEMBRANE DELIVERY SYSTEMS AND USES THEREOF

(71) Applicant: APOSENSE LTD., Petach Tikva (IL)

(72) Inventors: Ilan ZIV, Kfar Saba (IL); Hagit GRIMBERG, Herzliya (IL); Joel Moshe VAN GELDER, Jerusalem (IL)

(73) Assignee: APOSENSE LTD., Petach Tikva (IL)

(21) Appl. No.: 19/113,605

(22) PCT Filed: Sep. 21, 2023
(86) PCT No.: PCT/IL2023/051030

§ 371 Date: Mar. 20, 2025

Related U.S. Application Data
(60) Provisional application No. 63/408,888, filed on Sep. 22, 2022.
(60) Provisional application No. 63/436,642, filed on Jan. 2, 2023.
(60) Provisional application No. 63/436,644, filed on Jan. 2, 2023.

(51) Int. Cl. A61K 47/54

(52) U.S. Cl. CPC A61K 47/554; A61K 47/549

(57) ABSTRACT

The present invention is directed to oligonucleotide conjugates, pharmaceutical compositions comprising same and to methods for use thereof, such as for the treatment and/or prevention of a genetic disease, or a viral disease in a subject.

### CROSS-REFERENCE TO RELATED APPLICATIONS

This application claims the benefit of priority of U.S. Provisional Patent Application No. 63/408,888, titled “TRANS-MEMBRANE DELIVERY SYSTEMS AND USES THEREOF”, filed Sep. 22, 2022; of U.S. Provisional Patent Application No. 63/436,644, titled “TRANS-MEMBRANE DELIVERY SYSTEMS AND USES THEREOF”, filed Jan. 2, 2023, and of U.S. Provisional Patent Application No. 63/436,642, titled “TRANS-MEMBRANE DELIVERY SYSTEMS AND USES THEREOF”, filed Jan. 2, 2023. The contents of which are incorporated herein by reference in their entirety.

### REFERENCE TO AN ELECTRONIC SEQUENCE LISTING

The contents of the electronic sequence listing (APSN-P-012-PCT.xml; size: 125,321 bytes; and date of creation: Sep. 21, 2023) is herein incorporated by reference in its entirety.

### BACKGROUND OF THE INVENTION

A major challenge in the implementation of macromolecular compounds, (including viral vectors, bacterial vectors, single-stranded or double-stranded oligonucleotide, natural or modified RNA or DNA molecules, or combinations thereof, siRNA (small interfering RNA), siRNA substrates for the Dicer enzyme (dsiRNA), microRNA (miRNA), messenger RNA (mRNA) drugs, DNA sequences designed to serve as antisense oligonucleotides (ASO) in clinical practice, mainly relates to intracellular delivery and optimization of their binding to plasma proteins, especially to albumin.

While indicating an enormous potential to advance medicine, all these potentially breakthrough genetic drugs share one major limitation: being built of oligonucleotides, either natural or modified, and with their site of action being inside the cell (the cytoplasm or the nucleus), all these therapeutic agents must pass through the hydrophobic barriers of cell membranes. Indeed, to date, this robust delivery barrier holds back the entire field of genetic therapies and precludes its implementation as the medical practice of the future. Moreover, oligonucleotide drugs are large molecules (for example siRNA has molecular weight of ≈15,000 Daltons), and each molecule harbors numerous negative charges (phosphates). Taken together, delivery of siRNA across cell membrane is associated with a very large energetic cost.

Currently, there are two major strategies for gene delivery across biological barriers: viral vectors, and non-viral vectors. Each strategy poses its own substantial limitations: the viral vector strategy is limited by low transfection rates, limited bio-distribution, and low safety/marked toxicity of the viral particles. Among the non-viral approaches, cationic lipids and related liposomes used for siRNA delivery are also markedly limited by toxicity. There is therefore a great unmet need for novel, efficient, and safer modes of delivery of genetic material across cell membranes, based on novel mechanisms of action, with optimal binding to plasma proteins.

### SUMMARY OF THE INVENTION

The invention thus provides a conjugate, having the structure of general Formula (I):

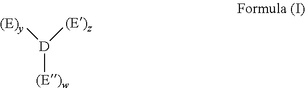

including pharmaceutically acceptable salts, hydrates, solvates and metal chelates thereof, wherein:
D is a compound selected from: single-stranded or double-stranded DNA, RNA, siRNA, dsiRNA, DNAzyme, ASO, a viral vector, a bacterial vector and any combinations thereof;
Each of y, z and w is an integer, independently selected from 0, 1, 2, 3 or 4, wherein at least one of y, z or w is different than 0;
E, E′, or E″ can be the same or different, each having independently a structure of general Formula (II)

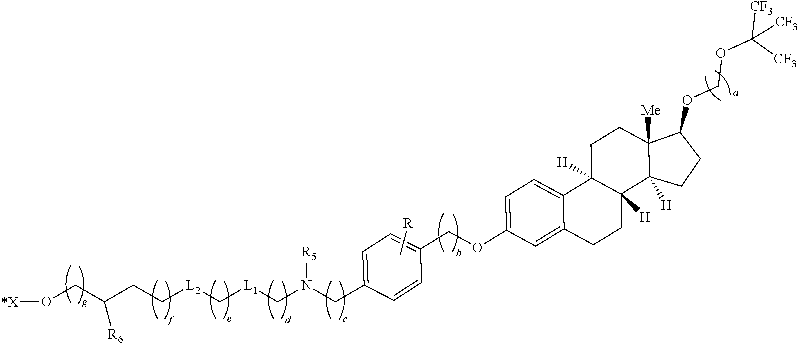

including pharmaceutically acceptable salts, hydrates, solvates and metal chelates thereof, wherein: X represents

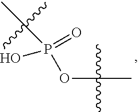 [SMILES: *OP(*)(=O)O]

a salt thereof, or both, or X is absent; each of a, b, c, d, e, f, g is an integer independently selected from 1, 2, 3, 4, 5, 6, 7, 8, 9, 10; R represents one or more substituents, each independently is selected from H, F, Cl, Br, I provided that at least one of the one or more substituents is F; R5 is H or a straight or branched C1-C5 alkyl; R6 is selected from H, hydroxy alkyl, and —(CH₂)ₙR′; wherein n is an integer selected from 1, 2, 3, 4, 5, 6, 7, 8, 9, 10, and wherein R′ is selected from a bond, H, and phosphate or a salt thereof; L1 is a linker selected from —NH—C(═O)—, —C(═O)NH—, —S—S— and —S—C(═O); L2 is a linker selected from —O—, —S—, —CH₂—; and * is the interaction/conjugation with D; Or wherein E, E′, or E″ can be the same or different, each having independently a structure of general Formula (III) including pharmaceutically acceptable salts, hydrates, solvates and metal chelates thereof, wherein: each of h, i, j, k is an integer independently selected from 1, 2, 3, 4, 5, 6, 7, 8, 9, 10; R7 is selected from H, hydroxy alkyl, and —(CH₂)ₙR′; wherein n is an integer selected from 1, 2, 3, 4, 5, 6, 7, 8, 9, 10, and wherein R′ is selected from a bond, H, and phosphate or a salt thereof; L3 is a linker selected from —NH—C(═O)—, —C(═O)NH—, —S—S— and —NH—C(═O); L4 is a linker selected from —O—, —S—, —CH₂—; * is the interaction/conjugation with D; and wherein X represents

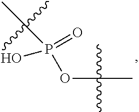 [SMILES: *OP(*)(=O)O]

a salt thereof, or both, or is absent

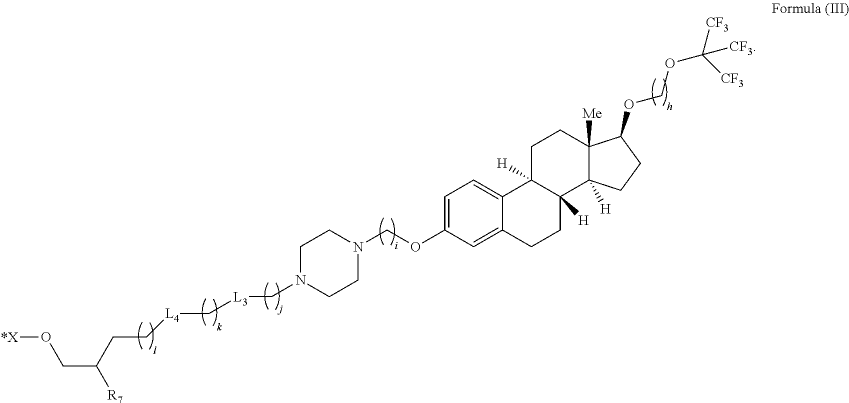

In some embodiments, R1-R4 is F. In some embodiments, R1, R2, R3 are H and R4 is F. In some embodiments, R1, R2, R4 are H and R3 is F. In some embodiments, R1, R3, R4 are H and R2 is F. In some embodiments, R2, R3, R4 are H and R1 is F. In some embodiments, R1 and R2 are H and R3 and R4 are F. In some embodiments, R3 and R4 are H and R1 and R2 are F. In some embodiments, R1, R2, R3 are F and R4 is H. In some embodiments, R1, R2, R4 are F and R3 is H. In some embodiments, R1, R3, R4 are F and R2 is H. In some embodiments, R2, R3, R4 are F and R1 is H.

In some embodiments, R5 is Me.

In some embodiments, R6 is —CH₂OH. In some embodiments, R6 is H.

In some embodiments, L1 is —S—S—. In some embodiments, L1 is —S—C(═O).

In some embodiments, L3 is —S—S—. In some embodiments, L3 is —NH—C(═O).

In some embodiments, R7 is —CH₂OH. In some embodiments, R7 is H.

In some embodiments, y is 0. In some embodiments, z is 0. In some embodiments, w is 0. In some embodiments, y and z are 0.

In some embodiments, D is a macromolecular drug. In some embodiments, D is an oligonucleotide drug comprising natural or modified oligonucleotide chains, and selected from siRNA, dsiRNA, mRNA, microRNA, DNAzyme, and ASO and any combinations thereof. In some embodiments, D is a viral vector. In some embodiments, D is a bacterial vector.

The invention further provides a pharmaceutical composition, comprising a conjugate as disclosed herein above and below.

The invention further provides a conjugate as disclosed herein above and below for use in gene therapy.

The invention further provides a conjugate as disclosed herein above and below for use in the treatment of hearing loss. The invention further provides a conjugate as disclosed herein above and below for use in the treatment of CNS diseases and disorders. The invention further provides a conjugate as disclosed herein above and below for use in cell and gene therapy. The invention further provides a conjugate as disclosed herein above and below for use in the treatment of cancer. The invention further provides a conjugate as disclosed herein above and below for use in inflammation bowel disease (IBD).

The invention further provides a precursor of a conjugate as disclosed herein above and below having general formula (II) wherein * is coupled to a protecting group.

The invention further provides a precursor of a conjugate as disclosed herein above and below having general formula (II) wherein OH is coupled to a protecting group.

In some embodiments, a precursor (i.e., one or more functional groups are connected to a protecting group) of the invention is a compound of the following structure:

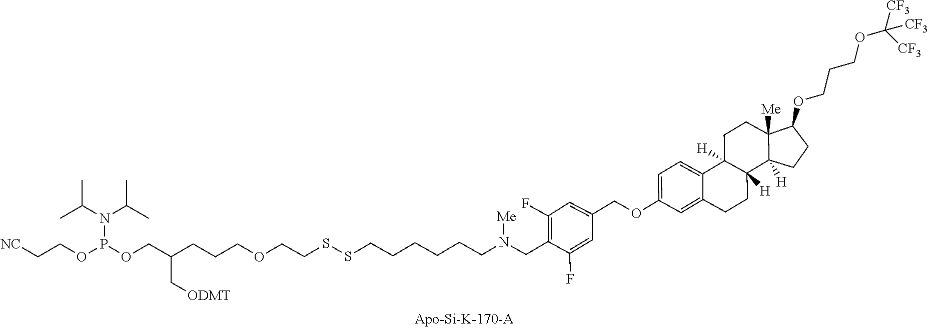

In some embodiments, a precursor (i.e., one or more functional groups are connected to a protecting group) of the invention is a compound of the following structure: Apo-Si-K170B, Apo-Si-K170C.

In some embodiments, a precursor (i.e., one or more functional groups are connected to a protecting group) of the invention is a compound of the following structure:

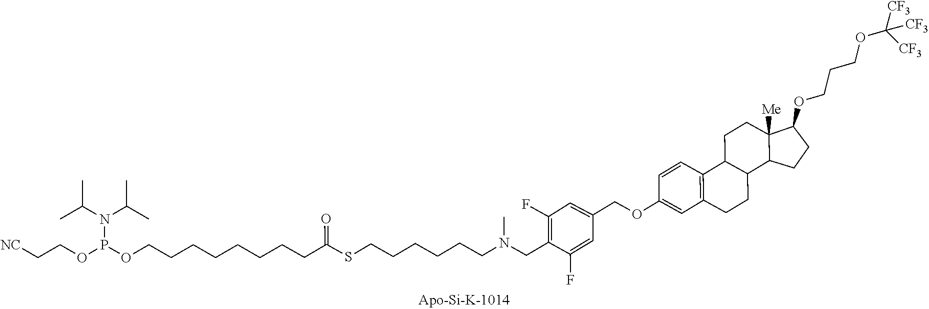 [SMILES: CC(C)N(C(C)C)P(OCCC#N)OCCCCCCCCC(=O)SCCCCCCN(C)Cc1c(F)cc(COc2ccc3c(c2)CCC2C3CC[C@@]3(C)C2CC[C@@H]3OCCCOC(C(F)(F)F)(C(F)(F)F)C(F)(F)F)cc1F]

In some embodiments, a precursor (i.e., one or more functional groups are connected to a protecting group) of the invention is a compound of the following structure:

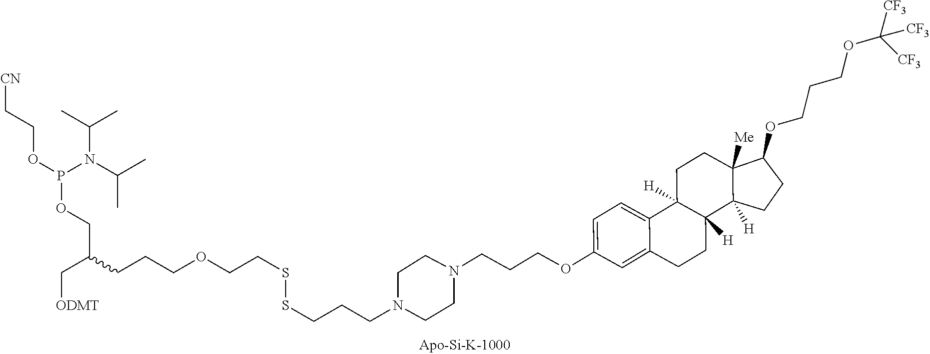

In some embodiments, a precursor (i.e., one or more functional groups are connected to a protecting group) of the invention is a compound of the following structure:

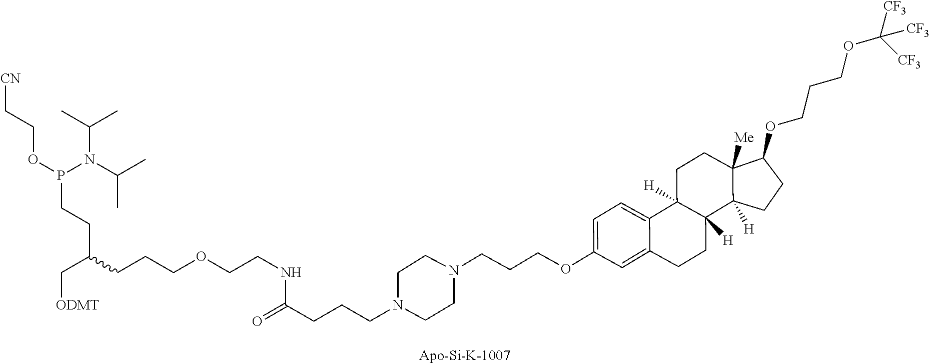

In some embodiments, a precursor (i.e.,one or more functional groups are connected to a protecting group) of the invention is a compound of the following structure:

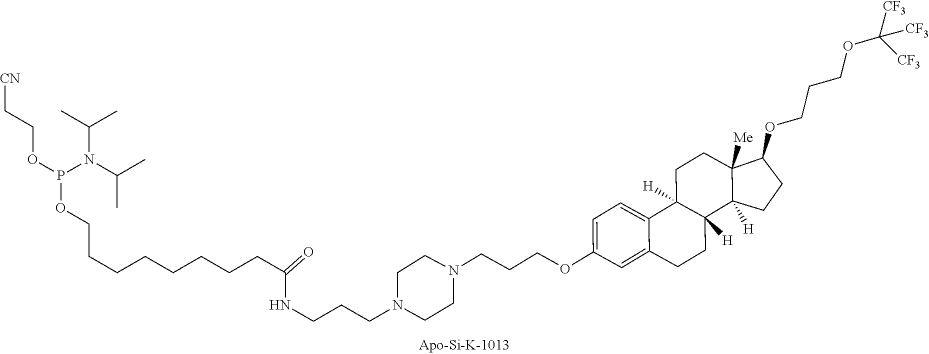 [SMILES: CC(C)N(C(C)C)P(OCCC#N)OCCCCCCCCC(=O)NCCCN1CCN(CCCOc2ccc3c(c2)CC[C@@H]2[C@@H]3CC[C@]3(C)[C@@H](OCCCOC(C(F)(F)F)(C(F)(F)F)C(F)(F)F)CC[C@@H]23)CC1]

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 demonstrates protein-free fraction of the exemplary conjugates of the invention and of a structurally similar analog (Apo-Si-S1) upon incubation with BSA.

FIG. 2 demonstrates that incubation of the Apo-Si-K-170-A, Apo-Si-K-170-B and Apo-Si-K-170-C conjugates with glutathione (GSH) (5 mM for 4 hours in 37° C.) led to a robust cleavage of the conjugates in the following order: Apo-Si-K-170-C>Apo-Si-K-170-A>Apo-Si-K-170-B>>Apo-Si-K-93-A.

FIGS. 3A-3D show EGFP silencing activity of Apo-Si-K-170-A, Apo-Si-K-170-B, Apo-Si-K-170-C and Apo-Si-K 941 conjugates in Hela-GFP cell line. FIG. 3A: 600 nM of the Apo-Si-K-170-A, Apo-Si-K-170-B and Apo-Si-K-170-C conjugates reduced EGFP expression to 49.9%, 83.1% and 70.8% of untreated control, respectively in the presence of complete medium, 10% serum in Hela-GFP cell line. FIGS. 3B-3D: dose dependent EGFP silencing activity of Apo-Si-K-170-A, Apo-Si-K-170-B, Apo-Si-K-170-C and Apo-Si-K 941 in different cell lines (serum free).

FIG. 4 shows that Apo-Si-K170A Conjugate significantly reduce the mRNA expression of epithelial sodium channel (ENaC) gene in the lungs, upon intratracheal (IT) administration to ICR mice in a dose response manner (100-200 ug/mice/dose).

FIG. 5 shows that the Apo-Si-K170A Conjugated to ENaC dsiRNA reduce the expression of ENaC gene in the mice lungs, specifically in comparison with Apo-Si-K170A Conjugated to control sequence, upon IT administration in mice.

FIG. 6 shows upon IT administration to mice, Apo-Si-K170A Conjugated to ENaC dsiRNA is local and didn't affect the ENaC expression in the kidney and the liver.

FIGS. 7A-7F show a comparison of Cy3 staining between Naked dsiRNA treated group following intracochlear (IC) administration at T+30 HOURS (Group 2)(FIGS. 7D-F) and Apo-Si-K170A dsiRNA conjugate treated group (IC) T+30 HOURS (Group 4) at the base of the cochlea (FIGS. 7A-C).

FIGS. 8A-8C are bar graphs showing down-regulation of PMP-22 target gene by Apo-Si-K1000 Construct. Cells were transfected with Apo-Si-K1000 Constructs. RNA was extracted from the cells 48 later and subjected to RT-qPCR analysis 8A. 3T3-NIH cells (N=5; n=2-10) 8B. Schwann cells S16 (N=2; n=2-4) 8C. HeLa cells (N=2; n=2-4).

FIG. 9 shows the results of the plaque assay in RSV infection mice model upon IT administration with Apo-Si-K170A Construct.

FIGS. 10A-10B show the oropharyngeal (10A) and BALF (10B) swab qRT-PCR results in AGM.

FIGS. 11A-11C show the SARS-CoV2 genomic qRT-PCR in AGM; Nasal swabs (11A), oropharyngeal (11B) and BALF (11C) results over time.

FIGS. 12A-12B show Apo-Si-K170A STAT6 down-regulates IL-4 (12A) & IL-13 (12B) cytokines in BALF of asthma mice model.

FIG. 13 shows the effect of Apo-Si-K170A STAT6 on total IgE in plasma of Asthma mice model.

FIG. 14 shows SPARC silencing efficiency of Apo-Si-K-170A-SPARC (leading conjugate) in vitro, compared to a non-related negative control (dsiDyn1i2 #1).

FIG. 15 shows Down-regulation of ENaC target gene at various concentrations of Apo-Si-K-170A-SPARC, as compared to a non-related negative control (K170A-Dyn1i2).

FIGS. 16A-16D show dose-dependent inhibition of Influenza A Virus in MDCK cells by Apo-Si-K170A-dsiRNA constructs. FIG. 16A: K170A-MF03-PB1, FIG. 16B: K170A-MF43-PB2, FIG. 16C: K170A-MF13-PB1, FIG. 16D: K170A-MF45-PB2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one aspect of the invention, there is provided a conjugate, having the structure of general Formula (I):

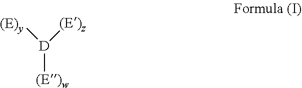

including pharmaceutically acceptable salts, hydrates, solvates and metal chelates thereof, wherein D is a polynucleic acid; each of y, z and w is an integer, independently selected from 0, 1, 2, 3 or 4, wherein at least one of y, z or w is different than 0; E, E′, or E″ can be the same or different, each having independently a structure of general Formula (I):

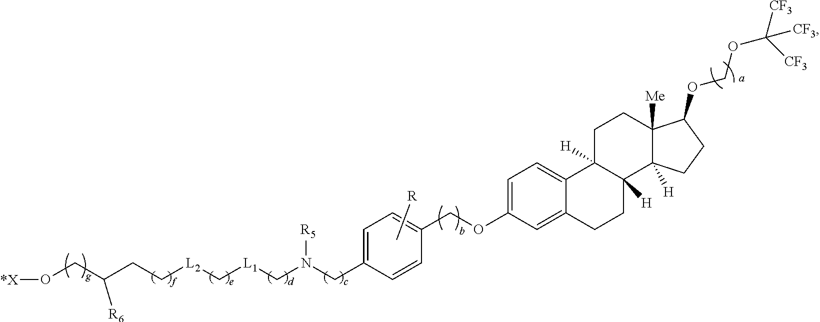

including pharmaceutically acceptable salts, hydrates, solvates and metal chelates thereof, wherein X represents

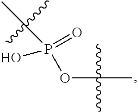 [SMILES: *OP(*)(=O)O]

a salt thereof, or both, or X is absent; each of a, b, c, d, e, f, g is an integer independently selected from 1, 2, 3, 4, 5, 6, 7, 8, 9, 10, or 1-10; R represents one or more substituents, each independently is selected from H, F, Cl, Br and I, provided that at least one of the one or more substituents is F; R5 is H or a straight or branched C1-C5 alkyl; R6 is selected from H, —(CH₂)ₙOH, hydroxy alkyl, —(CH₂)ₙR′, and —(CH₂)ₙO*; wherein n is an integer selected from 1, 2, 3, 4, 5, 6, 7, 8, 9, 10 or 1-10; and wherein R′ is selected from a bond, H, and phosphate and/or a salt thereof, L1 is a linker selected from (i) —X—C(═X)—, wherein each X independently represents N, NH, S, or O; and (ii) —NH—C(═O)—, —C(═O)NH—, S—C(═O)—, —S—S— and —S—C(═O); L2 is absent or is a linker selected from —O—, —S—, —CH₂—; and wherein * represents an attachment point or conjugation point to D. In some embodiments, R represents at least 2 substituents each independently selected from F, Cl, Br and I. In some embodiments, R represents 2 or 3 substituents, each independently selected from F, Cl, Br and I. In some embodiments, R represents 2 or 3 fluoro substituents.

In some embodiments, any one of E, E′, and E″ is independently bound to (i) a terminal site of the sequence of D (e.g. 3′ or 5′ of one or more polynucleotides), or to (ii) an interior site of the sequence of D. In some embodiments, the conjugate of the invention has a plurality of E, E′, or E″ moieties bound to a terminal site of the sequence of D. In some embodiments, the conjugate of the invention further comprises at least one of E, E′, or E″ bound to an interior site of the sequence of D.

In some embodiments, the conjugate of the invention is represented by general Formula (I), wherein D is a polynucleotide; wherein at least two of y, z or w is different than 0; and wherein R6 of at least one of E, E′, and E″ is —(CH₂)ₙO* (i.e. having two attachment points to D). In some embodiments, the conjugate of the invention is as described herein; wherein D is a polynucleotide; and the conjugate comprises two or more of E, E′, and E″ (e.g. 2, 3, or 4); wherein R6 of at least one of E, E′, and E″ is —(CH₂)ₙO* (i.e. is bound in the middle of the sequence of D), and wherein R6 of at least one additional E, E′, or E″ is selected from H, —(CH₂)ₙOH, hydroxy alkyl, and —(CH₂)ₙR′ (i.e. is bound to 3′ or 5′ terminus of the sequence of D). In some embodiments, the conjugate of the invention is as described herein; wherein D is a polynucleotide, and wherein the conjugate of the invention comprises two of E, E′, and E″ bound to 3′ or 5′ terminus of the sequence of D.

In some embodiments, the conjugate of the invention comprises 2 E, E′, or E″ moieties bound to D. In some embodiments, the conjugate of the invention comprises 3 E, E′, or E″ moieties bound to D. In some embodiments, the conjugate of the invention comprises 4 E, E′, or E″ moieties bound to D.

In some embodiments, each of E, E′, or E″ is independently represented by Formula (II1):

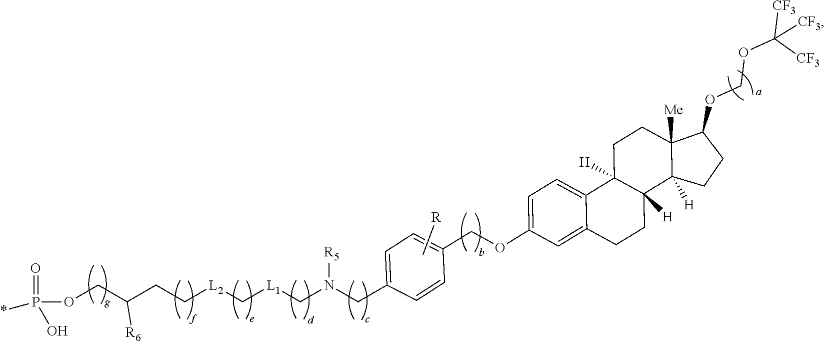

including any salt thereof; wherein a, b, c, d, e, f, g, R, R5, and R6 are as described hereinabove.

In some embodiments, each of E, E′, or E″ is independently represented by Formula (IIa):

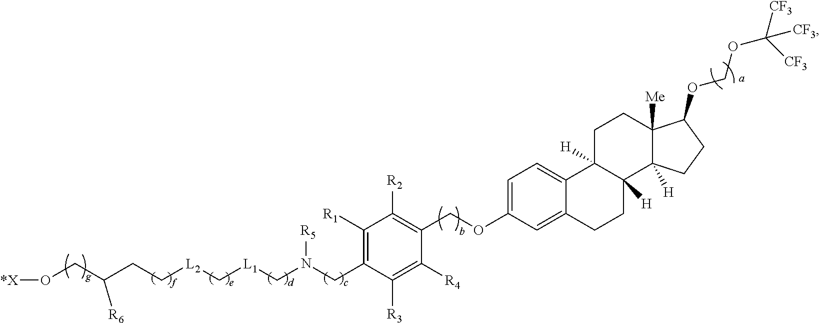

wherein each R1-R4 is independently selected from H, F, Cl, Br, I; wherein at least one of R1-R4 is not H. In some embodiments, each of R1, R2, R3, and R4 is independently selected from H, F, Cl, Br, I; wherein at least one of R1, R2, R3, and R4 is F.

In some embodiments, each of E, E′, or E″ is independently represented by Formula (IIa1):

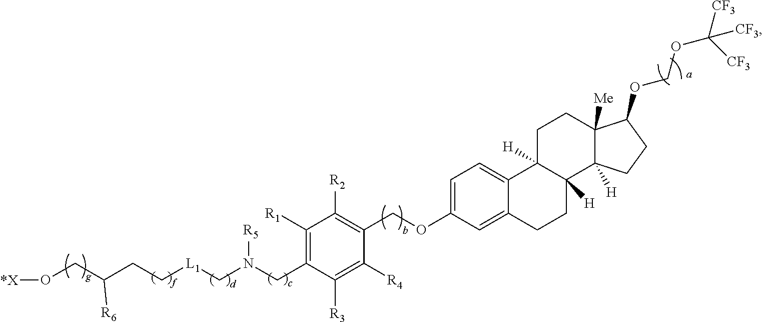

wherein each R1-R4 is independently selected from H, F, Cl, Br, and I; wherein at least one of R1-R4 is not H, wherein R5 is an alkyl; wherein X is absent or represents

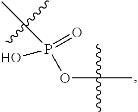 [SMILES: *OP(*)(=O)O]

a salt thereof, or both; wherein a is 3; wherein b is between 1 and 5; c is between 1 and 3; and each of d, e, f and g is between 1 and 10; and wherein X, R6, and L1 are as described hereinabove.

In some embodiments, each of E, E′, or E″ is independently represented by any one of Formulae (II)-(IIa1), wherein L1 is —X—C(═X)—, and wherein L2 is —O—, —S— or is absent. In some embodiments, each of E, E′, or E″ is independently represented by any one of Formulae (II)-(IIa1), wherein L1 is —X—C(═X)—; wherein a is 3; wherein b is 1; c is 1; and each of d, e, f and g is between 1 and 10; and wherein L2 is absent. In some embodiments, each of E, E′, or E″ is independently represented by any one of Formulae (II)-(IIa1), wherein L1 is selected from —NH—C(═O)—, —C(═O)NH—, S—C(═O)— and —S—C(═O); wherein a is 3; wherein b is 1; c is 1; and each of d, e, f and g is between 1 and 10; and wherein L2 is absent.

In some embodiments, the conjugate of the invention comprises one or more E, E′, or E″ moieties, wherein each of E, E′, and E″ is independently represented by any one of Formulae (II)-(IIa1), wherein L1 is selected from —NH—C(═O)—, —C(═O)NH—, S—C(═O)— and —S—C(═O); wherein a is 3; wherein b is 1; c is 1; and each of d, e, f and g is between 1 and 10; and wherein L2 is absent; and wherein the conjugate is for systemic administration (e.g. in a form of a pharmaceutical composition formulated for systemic administration.) In some embodiments, the conjugate of the invention comprises at least 2 E, E′, or E″ moieties (e.g. 2 or 3), wherein each of E, E′, and E″ is independently represented by any one of Formulae (II)-(IIa1), wherein L1 is selected from —NH—C(═O)—, —C(═O)NH—, S—C(═O)— and —S—C(═O); wherein a is 3; wherein b is 1; c is 1; and each of d, e, f and g is between 1 and 10; and wherein L2 is absent.

and wherein the conjugate is for systemic administration (e.g. in a form of a pharmaceutical composition formulated for systemic administration.) In some embodiments, each of E, E′, or E″ is independently represented by Formula (II), Formula (II1), Formula (IIa), or by Formula (IIa1), wherein L1 is —S—S—, and wherein L2 is —O—, or —S—.

In some embodiments, each of E, E′, or E″ is independently represented by Formula (IIb):

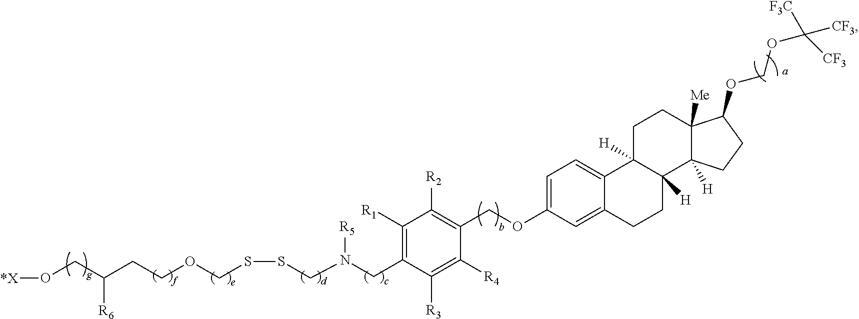

wherein: a is between 1 and 5; b is between 1 and 3; c is between 1 and 3; and each of d, e, f and g is between 1 and 10. In some embodiments, each of a, b and c is between 1 and 3, and each of d, e, f and g is between 1 and 10.

In some embodiments, each of E, E′, or E″ is independently represented by Formula (IIb), wherein a is 3; wherein b is 1; c is between 1 and 3; and each of d, e, f and g is between 1 and 10. In some embodiments, the conjugate of the invention comprises one or more E, E′, or E″ moieties, wherein each of E, E′, and E″ is independently represented by Formula (IIb), wherein a is 3; wherein b is 1; c is 1; and each of d, e, f and g is between 1 and 10; and wherein the conjugate is for local administration (e.g. in a form of a pharmaceutical composition formulated for local administration). In some embodiments, the conjugate of the invention comprises at least 2 E, E′, or E″ moieties (e.g. 2 or 3), wherein each of E, E′, and E″ is independently represented by Formula (IIb), wherein a is 3; wherein b is 1; c is 1; and each of d, e, f and g is between 1 and 10; and wherein the conjugate is for local administration (e.g. in a form of a pharmaceutical composition formulated for local administration.)

In some embodiments, R1 and R3 are F. In some embodiments, R1 and R3 are F.

In some embodiments, R2 and R4 are F. In some embodiments, R1, R3 and one of R4 and R2 are F. In some embodiments, RI and R4 are F. In some embodiments, R2 and R3 are F.

In some embodiments, each of E, E′, or E″ is independently represented by Formula (IIc):

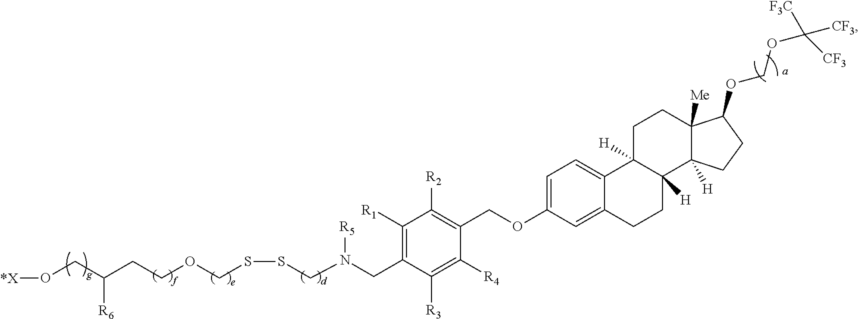

wherein a is between 1 and 5, and each of d, e, f and g is between 1 and 10.

In some embodiments, each of E, E′, or E″ is independently represented by Formula IId:

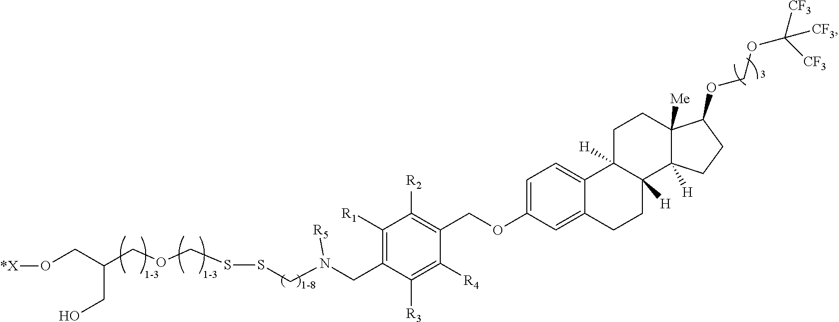

### by Formula IIe:

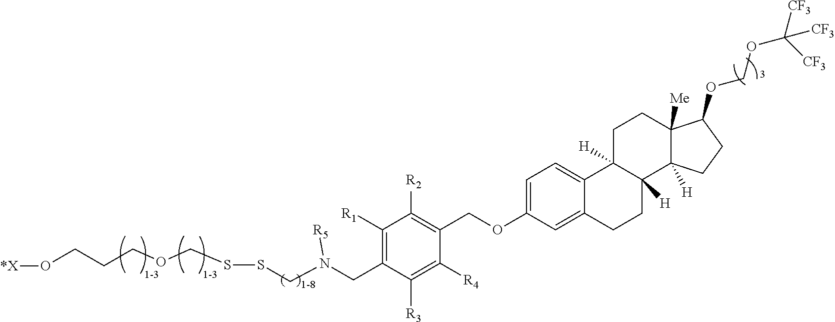

### or by Formula IIf:

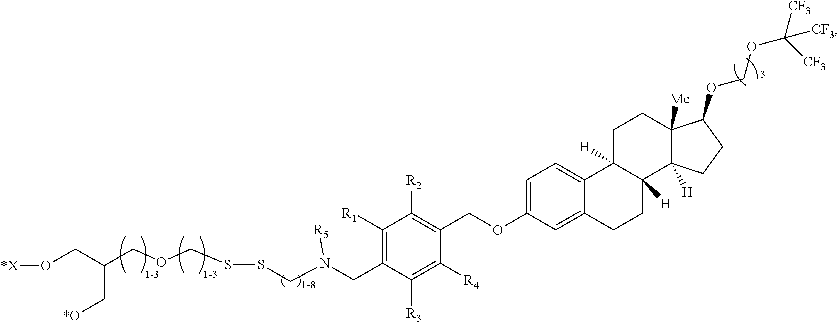

wherein R1-R4 are as described hereinabove, and wherein R5 is a C1-C5 alkyl.

In some embodiments, each of E, E′, or E″ is independently represented by Formula (IIIa):

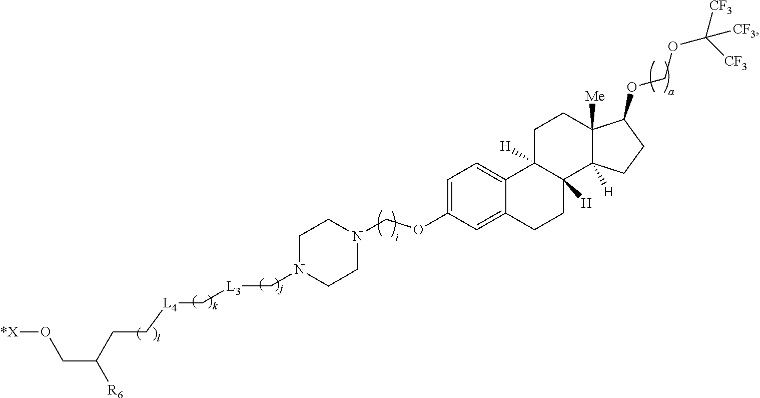

including pharmaceutically acceptable salts, hydrates, solvates and metal chelates thereof, wherein: each of i, j, k and l is an integer independently selected from 1, 2, 3, 4, 5, 6, 7, 8, 9, 10; L3 is a linker selected from (i) —X—C(═X)—, wherein each X independently represents N, NH, S, or O; and (ii) —NH—C(═O)—, —C(═O)NH—, S—C(═O)—, —S—S— and —S—C(═O); L4 is a linker selected from —O—, —S—, —CH₂— or is absent; wherein a, R6, L1 and L2 are as described hereinabove; and wherein * is the attachment point or conjugation point to D.

In some embodiments, each of E, E′, or E″ is independently represented by Formula IIIa1:

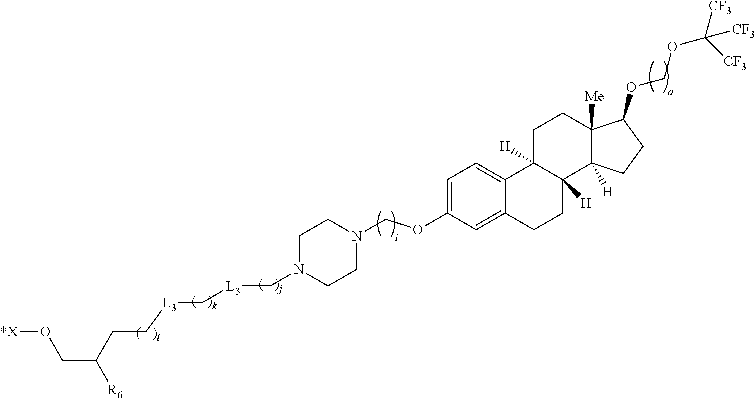

including pharmaceutically acceptable salts, hydrates, solvates and metal chelates thereof, wherein: each of i, j, k and l is an integer independently selected from 1, 2, 3, 4, 5, 6, 7, 8, 9, 10; each L3 is independently a linker selected from —O—, —S—, —CH₂—, —NH—C(═O)—, —C(═O)NH—, —S—S—, S—C(═O)— and —S—C(═O), optionally wherein one L3 is absent; wherein a, R6, L1 and L2 are as described hereinabove; and wherein * is the attachment point or conjugation point to D. The integers R6 and R7 are used herein interchangeably.

In some embodiments, each of E, E′, or E″ is independently represented by Formula (IIIb):

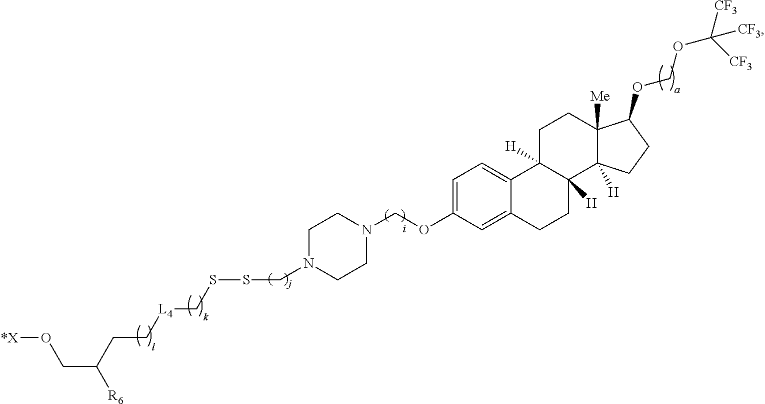

### or by Formula IIIc:

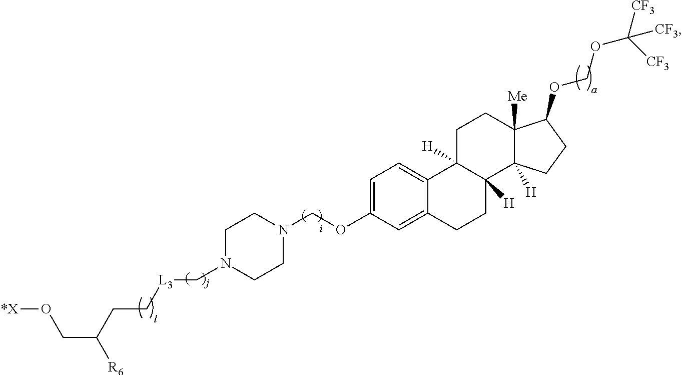

wherein i, j, k, l, a, R6, L3 are as described hereinabove. In some embodiments, each of E, E′, or E″ is independently represented by Formula (IIIa-c) wherein each of a, i, j and k is independently between 1 and 5, or between 1 and 3.

In some embodiments, each of E, E′, or E″ is independently represented by Formula (IIIb), wherein i is 1, wherein j and k is independently between 1 and 5; and wherein L4 is absent. In some embodiments, the conjugate of the invention comprises one or more E, E′, or E″ moieties, wherein each of E, E′, and E″ is independently represented by Formula (IIIb), wherein i is 1, wherein j and k is independently between 1 and 5; and wherein L4 is absent; and wherein the conjugate is for local administration (e.g. in a form of a pharmaceutical composition formulated for local administration). In some embodiments, the conjugate of the invention comprises at least 2 E, E′, or E″ moieties (e.g. 2 or 3), wherein each of E, E′, and E″ is independently represented by Formula (IIIb), wherein i is 1, wherein j and k is independently between 1 and 5; and wherein L4 is absent; and wherein the conjugate is for local administration (e.g. in a form of a pharmaceutical composition formulated for local administration.)

In some embodiments, each of E, E′, or E″ is independently represented by Formula (IIIc), wherein i is 1, wherein j and k is independently between 1 and 5; and wherein L3 is selected from —NH—C(═O)—, —C(═O)NH—, S—C(═O)— and —S—C(═O). In some embodiments, the conjugate of the invention comprises one or more E, E′, or E″ moieties, wherein each of E, E′, and E″ is independently represented by Formula (IIIc), wherein i is 1, wherein j and k is independently between 1 and 5; and wherein L3 is selected from —NH—C(═O)—, —C(═O)NH—, S—C(═O)— and —S—C(═O); and wherein the conjugate is for systemic administration (e.g. in a form of a pharmaceutical composition formulated for systemic administration.) In some embodiments, the conjugate of the invention comprises at least 2 E, E′, or E″ moieties (e.g. 2 or 3), wherein each of E, E′, and E″ is independently represented by Formula (IIIc), wherein i is 1, wherein j and k is independently between 1 and 5; and wherein L3 is selected from —NH—C(═O)—, —C(═O)NH—, S—C(═O)— and —S—C(═O); and wherein the conjugate is for systemic administration (e.g. in a form of a pharmaceutical composition formulated for systemic administration.)

In some embodiments, each of E, E′, or E″ is independently represented by Formula (IIId):

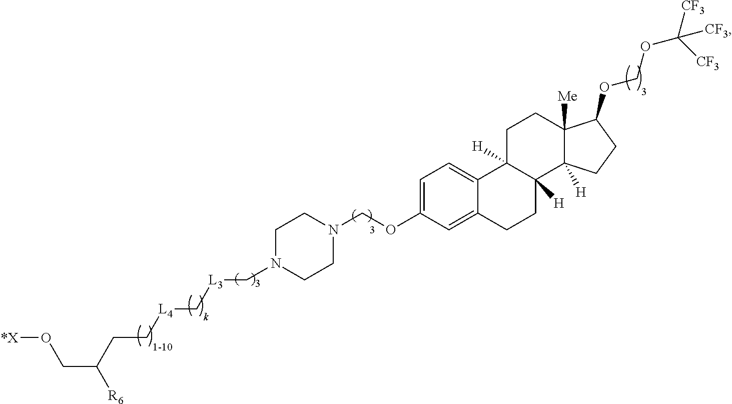

wherein L3, L4, X and R6 are as described above.

In some embodiments, each of E, E′, or E″ is independently selected from:

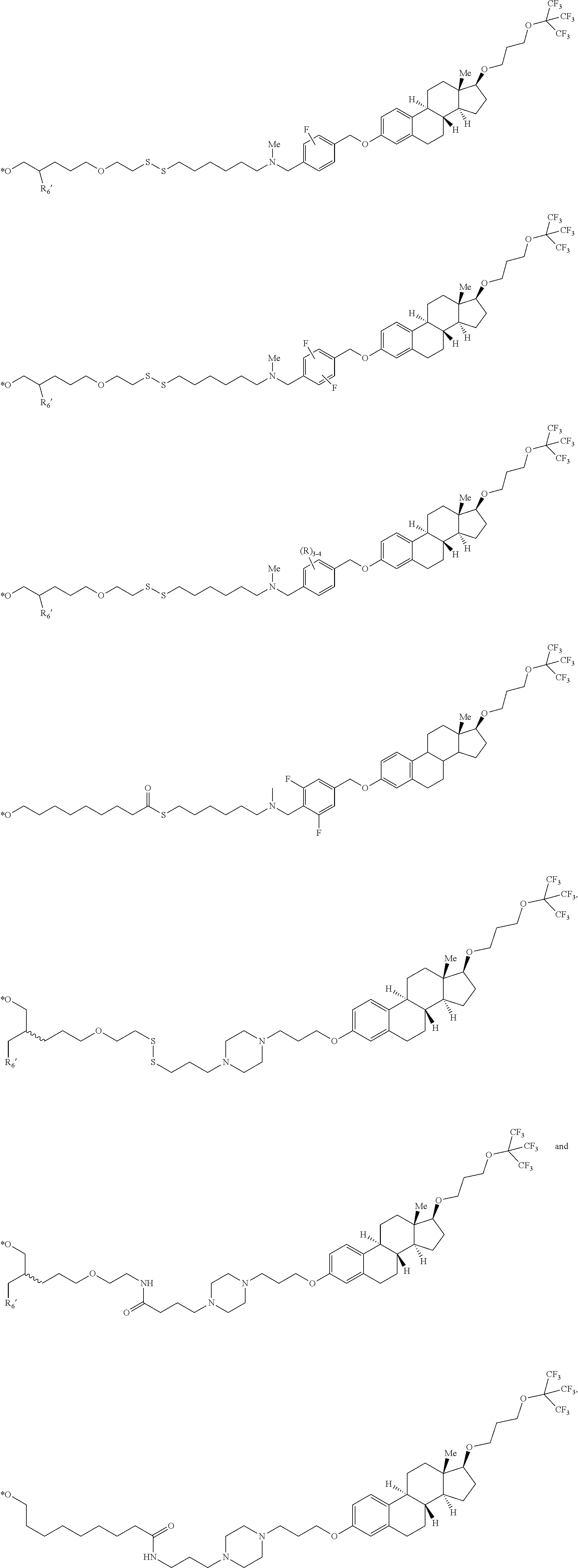

wherein R6′ is OH, phosphate or O*, and wherein* is the attachment point or conjugation point to D.

In some embodiments, D is a polynucleic acid molecule (polynucleotide). In some embodiments, D is DNA. In some embodiments, the D is RNA. In some embodiments, the polynucleic acid molecule is an oligonucleotide. In some embodiments, D is an aptamer. In some embodiments, D is a primer. In some embodiments, D is an antisense oligonucleotide. In some embodiments, D is a regulatory RNA. In some embodiments, D is plasmid. In some embodiments, D is an expression vector. In some embodiments, the vector is configured to expresses in a target cell. In some embodiments, D is gene therapy. In some embodiments, the polynucleic acid molecule comprises an open reading frame. In some embodiments, the open reading frame encodes a therapeutic protein. Methods of conjugating polynucleic acid molecules to chemical and amino acid linkers are well known in the art and any such method may be employed. In some embodiments, the polynucleic acid molecule comprises a nuclear localization signal (NLS).

The term “polynucleic acid” is well known in the art. A “polynucleic acid” as used herein will generally refer to a molecule (i.e., a strand) of DNA, RNA or a derivative or analog thereof, comprising a plurality (e.g. at least 2) nucleobases. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C), and further encompasses chemically modified nucleobases or nucleotides, such as O-methylated nucleotides, N-methylated nucleotides, phosphorothioated nucleotides, backbone modified nucleotides (LNA, morpholino).

The terms “polynucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating polynucleic acids, fragments of genomic DNA or RNA, degraded nucleic acids, ribozymes, viral RNA or DNA, polynucleic acids of infectious origin, amplification products, modified nucleic acids, plasmidical or organellar nucleic acids and artificial nucleic acids such as oligonucleotides.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

The mRNA, as provided herein, comprises at least one (one or more) ribonucleic acid (RNA) polynucleotide having an open reading frame encoding at least one polypeptide of interest.

In some embodiments, the polynucleotide is or comprises therapeutic polynucleotide. As used herein, the term “therapeutic polynucleotide” refers to a polynucleotide sequence that encodes a therapeutic protein, or is complementary to a sequence of interest (a mutant gene). A therapeutic polynucleotide having a complementary sequence to a mutant gene is also referred to herein as “an inhibitory nucleic acid”.

Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic polynucleotide may be useful for the treatment of the following diseases and conditions: bacterial infections, viral infections, parasitic infections, cell proliferation disorders, genetic disorders, and autoimmune disorders.

Thus, the polynucleotide of the invention can be used as therapeutic or prophylactic agents. They are provided for use in medicine. For example, the polynucleotide described herein can be administered to a subject, wherein the polynucleotides are translated in vivo to produce a therapeutic peptide.

In some embodiments, the therapeutic polynucleotide comprises an inhibitory nucleic acid. In some embodiments, the inhibitory nucleic acid is an antisense oligonucleotide.

As used herein, an “antisense oligonucleotide” refers to a nucleic acid sequence that is reversed and complementary to a DNA or RNA sequence.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory nucleic acid has one or more chemical modifications to the backbone or side chains. In some embodiments, the inhibitory nucleic acid has at least one locked nucleotide, and/or has a phosphorothioate backbone.

Non-limiting examples of inhibitory nucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acid is an RNA interfering molecule (RNAi). In some embodiments, the RNAi is or comprises double stranded RNA (dsRNA).

As used herein “an interfering RNA” refers to any double stranded or single stranded RNA sequence, capable-either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein. In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

As used herein, the terms “oligonucleotide” and “polynucleotide” refer to a polymer/oligomer containing nucleotides as repeating units. Usually, oligonucleotide contains 2-100 bases. Usually, polynucleotide contains 2-1000 bases. In another embodiment, the term “polynucleotide” refers to a molecule comprising 5-1000, 5-200, 5-300, 5-500, 5-700, 5-5000, 5-1000, 20-100, 20-1000, 50-200, 50-500, 50-1000, 50-100, bases, including any range between. In another embodiment, the term “oligonucleotide” refers to a molecule comprising 5-100 bases. In another embodiment, the term “oligonucleotide” refers to a molecule comprising 5-80 bases. In another embodiment, the term “oligonucleotide” refers to a molecule comprising 5-40 bases. In another embodiment, the term “oligonucleotide” refers to a molecule comprising 50-100 bases. In another embodiment, the term “oligonucleotide” refers to a molecule comprising 20-70 bases. In another embodiment, the term “oligonucleotide” refers to a molecule comprising 5-30 bases. In another embodiment, the term “oligonucleotide” refers to a molecule comprising 5-25 bases. In another embodiment, the term “oligonucleotide” refers to a molecule comprising 10-50 bases, 20-50 bases, 5-50 bases, 10-100 bases, including any range between.

The term “expression” as used herein refers to the biosynthesis of a gene product, including the transcription and/or translation of said gene product. Thus, expression of a nucleic acid molecule may refer to transcription of the polynucleic acid fragment (e.g., transcription resulting in mRNA or other functional RNA) and/or translation of RNA into a precursor or mature protein (polypeptide).

Expressing of a gene within a cell is well known to one skilled in the art and herein its delivery may be performed by a method of the invention or using a composition of the invention. In some embodiments, the gene is in an expression vector such as plasmid or viral vector. The vector may be a viral vector. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene or open reading frame is operably linked to a promoter or other regulatory element. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell by a method of the invention). In some embodiments, the regulatory element or promoter is active in a target cell.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

The term “bioactive” refers to a molecule or agent that exerts an effect on a cell or tissue. Representative examples of types of bioactive agents include therapeutics, vitamins, electrolytes, amino acids, peptides, polypeptides, proteins, enzymes, carbohydrates, lipids, polysaccharides, nucleic acids, nucleotides, polynucleotides, glycoproteins, lipoproteins, glycolipids, glycosaminoglycans, proteoglycans, growth factors, differentiation factors, hormones, neurotransmitters, prostaglandins, immunoglobulins, cytokines, and antigens. Various combinations of these molecules can be used. Examples of cytokines include macrophage derived chemokines, macrophage inflammatory proteins, interleukins, tumor necrosis factors. Examples of proteins include fibrous proteins (e.g., collagen, elastin) and adhesion proteins (e.g., actin, fibrin, fibrinogen, fibronectin, vitronectin, laminin, cadherins, selectins, intracellular adhesion molecules, and integrins). In various cases, the bioactive agent may be selected from fibronectin, laminin, thrombospondin, tenascin C, leptin, leukemia inhibitory factors, RGD peptides, anti-TNFs, endostatin, angiostatin, thrombospondin, osteogenic protein-1, bone morphogenic proteins, osteonectin, somatomedin-like peptide, osteocalcin, interferons, and interleukins. In some embodiments, the bioactive agent includes a growth factor, differentiation factor, or a combination thereof.

In some embodiments, the conjugate of the invention is characterized by any one of: increased gene expression modulation activity (e.g. gene downregulation or upregulation) within a subject, or within a cell; increased intracellular release of D (via cleavage of L1 or L3 moiety); increased free conjugate fraction (i.e. conjugate which is not bound to a protein, or any other biopolymer) in blood, serum or any other biological fluid; and recued serum protein binding. In some embodiments, the conjugate of the invention is characterized by increased gene expression modulation activity in-vitro in the presence of serum. The terms “increased” and “reduced” including any grammatical form thereof encompass at least 20%, 30%, 40%, 50%, at least 60%, at least 70%, at least 80%, at least 85%, at least 90%, at least 92%, at least 95%, at least 97%, at least 99%, at least 100%, at least 200%, at least 1000%, at least 10000%, or between 20 and 500%, between 50 and 1000% reduction or increase respectively, as compared to a control. In some embodiments, the control is a conjugate according to general Formula (I) comprising a scrambled sequence of D. In some embodiments, the control is a conjugate according to general Formula (I) comprising the same sequence of D; wherein E, E′, or E″ are structural analogs of E, E′, or E″ moieties disclosed herein; and wherein the structural analogs are not according to any one of the Formulae or structure presented herein. In some embodiments, the structural analog is Apo-Si-S1, or Apo-Si-K-93A. In some embodiments, the structural analog is Apo-K-160-A (see Examples section).

In some embodiments, the conjugate of the invention and/or a pharmaceutically acceptable salt thereof is formulated in a form of a pharmaceutical composition. In some embodiments, the pharmaceutical composition comprises one or more conjugate of the invention and a pharmaceutically acceptable carrier, excipient or adjuvant. In some embodiments, the pharmaceutical composition comprises a therapeutically effective amount of one or more conjugate of the invention.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, glycols, such as propylene glycol, polyols such as glycerin, sorbitol, mannitol, and polyethylene glycol; esters such as ethyl oleate and ethyl laurate, pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, stearic acid, magnesium stearate, calcium sulfate, polyols, pyrogen-free water, isotonic saline, phosphate buffer solutions, as well as other non-toxic pharmaceutically compatible substances used in other pharmaceutical formulations. Wetting agents and lubricants such as sodium lauryl sulfate, as well excipients, stabilizers, antioxidants, and preservatives may also be present. Any non-toxic, inert, and effective carrier may be used to formulate the compositions contemplated herein.

The carrier may comprise, in total, from about 0.1% to about 99.99999% by weight of the pharmaceutical compositions presented herein.

In some embodiments, the pharmaceutical composition comprises a therapeutically effective amount of the conjugate of the invention. In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to determine useful doses more accurately in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)]. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, there is provided a method for preventing or treating a genetic disease in a subject in need thereof and/or for reducing at least one symptom of a disease associated with a mutant gene, comprising administering to the subject a therapeutically effective amount of the pharmaceutical composition of the invention. In some embodiments, therapeutically effective amount is sufficient for reduction of at least one symptom, or for reduction in the severity and/or inhibition of the progression of a disease, disorder, or condition as described hereinabove. In some embodiments, therapeutically effective amount is sufficient to inhibit translation of the mutant gene. In some embodiments, therapeutically effective amount is sufficient to inhibit transcription of the mutant gene. In some embodiments, the genetic disease is associated with abnormal (e.g. increased) expression of a specific gene, as compared to a control. In some embodiments, abnormal expression comprises increased gene expression by at least 2 times, at least 5 times, at least 10 times, or more including any range between, compared to a healthy individual with normal expression of the specific gene.

In some embodiments, “substantially reduce” including any grammatical form thereof encompasses at least 20%, 30%, 40%, 50%, at least 60%, at least 70%, at least 80%, at least 85%, at least 90%, at least 92%, at least 95%, at least 97%, at least 100%, at least 200%, at least 1000%, at least 10000% reduction of transcription and/or translation of the mutant gene within the subject, compared to a control.

In some embodiments, therapeutically effective amount is sufficient to substantially reduce or completely inhibit any of the biological activity(s) of a cell comprising the mutant gene (e.g. cell proliferation, metabolism, etc.).

In some embodiments, therapeutically effective amount is sufficient to substantially reduce or completely eliminate viral load within the subject. In some embodiments, therapeutically effective amount is sufficient to substantially reduce or completely eliminate viral proliferation within the subject.

In some embodiments, “substantially reduce” including any grammatical form thereof encompasses at least 20%, 30%, 40%, 50%, at least 60%, at least 70%, at least 80%, at least 85%, at least 90%, at least 92%, at least 95%, at least 97%, at least 99%, reduction of the viral load within the subject, compared to a control.

In some embodiments, “substantially reduce” including any grammatical form thereof encompasses at least 20%, 30%, 40%, 50%, at least 60%, at least 70%, at least 80%, at least 85%, at least 90%, at least 92%, at least 95%, at least 97%, at least 99%, at least 100%, at least 200%, at least 1000%, at least 10000% reduction of the viral proliferation within the subject, compared to a control. In some embodiments, “substantially reduce” including any grammatical form thereof encompasses at least 20%, 30%, 40%, 50%, at least 60%, at least 70%, at least 80%, at least 85%, at least 90%, at least 92%, at least 95%, at least 97%, at least 99%, at least 100%, at least 200%, at least 1000%, at least 10000% reduction of the expression of at least one viral gene within the subject, compared to a control.

In some embodiments, the control comprises an untreated subject afflicted with the disease. In some embodiments, the control comprises an untreated subject having the mutant gene. In some embodiments, the control comprises an untreated subject afflicted with a disease or disorder associated with the mutant gene. In some embodiments, the control comprises an untreated subject having abnormal expression of the specific gene.

In some embodiments, administering comprises local or systemic administration.

In some embodiments, administering comprises intradermal, intravenous, intramuscular, intralesional, subcutaneous, parenteral, intraventricular, intrathecal, and any other mode of injection as known in the art. The route of administration of the pharmaceutical composition will depend on the disease or condition to be treated. Additional routes of administration include, but are not limited to buccal administration, oral administration, topical, rectal, vaginal, sublingual, nasal, ophthalmic, transdermal, subcutaneous, intramuscular, intraperitoneal, intrathecal, and pulmonary administration.

In some embodiments, the method comprises administering the pharmaceutical composition of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

In some embodiments, the method comprises administering the pharmaceutical composition of the invention to the subject at a daily dosage (e.g. once, twice or tree-times a day) of 0.05 to 50 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 0.8 to 25 mg/kg, 0.8 to 3 mg/kg, 0.8 to 10 mg/kg, 0.8 to 15 mg/kg, 0.8 to 5 mg/kg, 3 to 5 mg/kg, 3 to 10 mg/kg, 2 to 10 mg/kg, 1 to 2 mg/kg, 2 to 5 mg/kg, 5 to 10 mg/kg, 10 to 15 mg/kg, 15 to 20 mg/kg including any range or value therebetween. In some embodiments, the daily dose can be extrapolated from the in-vivo data, such as the results presented in Examples section (e.g. Example 4).

It should be apparent to one skilled in the art, that for example in-vitro and in-vivo assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the subject is afflicted with a disease or disorder comprising a viral disease, cancer, a genetic disease, a CNS disease, an inflammatory disease, a lung disease, or any combination thereof.

In some embodiments, the pharmaceutical composition is formulated for systemic administration. In some embodiments, the pharmaceutical composition is formulated for local administration. In some embodiments, the pharmaceutical composition is formulated for intravenous administration. In some embodiments, the pharmaceutical composition is formulated for administration to a subject.

As used herein, the term “pharmaceutically acceptable salt” refers to any non-toxic salt of the conjugate of the present invention that, upon administration to a subject, e.g., a human, is capable of providing, either directly or indirectly, a compound of this invention or a therapeutically active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases.

Non-limiting examples of pharmaceutically acceptable salts include but are not limited to: alkali metal salt, earth alkali metal salt, acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

Additional examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

In some embodiments, the conjugate of the invention is present in the pharmaceutical composition is of pharmaceutical grade purity, i.e., is characterized by a chemical purity of at least about 90%, at least about 95%, greater than 95%, or greater than 99%.

In some embodiments, the pharmaceutical composition is for use in the treatment of a disease or disorder in a subject in need thereof. In some embodiments, the pharmaceutical composition is for use in the reduction of at least one symptom associated with the disease or disorder. In some embodiments, the disease or disorder is a genetic disease.

The term “genetic disease” encompasses a disease associated with any abnormal expression and/or mutation of one or more genes.

In some embodiments, non-limiting examples of genetic diseases comprise but are not limited to a proliferative disease (e.g. cancer), an inflammatory disease (e.g. IBD, rheumatoid arthritis), CF, hearing loss, and CMT1A. In some embodiments, the conjugate of the invention and/or a pharmaceutically acceptable salt thereof is formulated in a form of a pharmaceutical composition for treatment or prevention of a respiratory viral disease in a subject in need thereof. In some embodiments, the pharmaceutical composition comprises one or more conjugate of the invention and a pharmaceutically acceptable carrier, excipient or adjuvant. In some embodiments, the pharmaceutical composition comprises a therapeutically effective amount of one or more conjugate of the invention. In some embodiments, the pharmaceutical composition is for use in the treatment or prevention of a disease or disorder associated with a respiratory viral infection, in a subject in need thereof.

In some embodiments, respiratory viruses are viruses such as Adenovirus, Coronavirus HKU1, Coronavirus NL63, Coronavirus 229E, Coronavirus OC43, Severe Acute Respiratory Syndrome Coronavirus 2 (SARS CoV 2), Human Metapneumovirus, Human Rhinovirus/Enterovirus, Influenza A, Influenza A/H, Influenza A/H3, Influenza A/H1-2009, Influenza B, Parainfluenza Viruses 1-4, Respiratory Syncytial Virus.

In some embodiments, there is provided a method for preventing or treating a respiratory viral infection in a subject in need thereof and/or for reducing at least one symptom of a disease associated with the respiratory viral infection, comprising administering to the subject a therapeutically effective amount of the pharmaceutical composition of the invention. In some embodiments, therapeutically effective amount is sufficient for reduction of at least one symptom, or for reduction in the severity and/or inhibition of the progression of a disease, disorder, or condition as described hereinabove. In some embodiments, therapeutically effective amount is sufficient to inhibit virulence of the respiratory virus. In some embodiments, therapeutically effective amount is sufficient to substantially reduce or completely inhibit any of the biological activity(s) of the respiratory virus, such as replication (e.g. RNA replication), transcription, translation, proliferation, etc.

In some embodiments, therapeutically effective amount is sufficient for reducing viral load within the subject, or for arresting viral proliferation, wherein the subject is afflicted with a respiratory viral disease.

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range e.g., “21-100”, is stated herein, it implies that the group, in this case the alkyl group, may contain 21 carbon atom, 22 carbon atoms, 23 carbon atoms, etc., up to and including 100 carbon atoms.

In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic, polycyclic (bi- or tri-cyclic), mixed- or fused-ring polycyclic (i.e. rings which share adjacent pairs of carbon atoms) groups having a completely conjugated pi-electron system. The aryl group may be substituted or unsubstituted, as indicated herein. The term “aryl” further encompasses heteroaromatic ring(s), such as monocyclic, polycyclic (bi- or tri-cyclic), mixed- or fused-ring polycyclic heteroaromatic rings.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine, or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “mercapto” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amino” describes a —NR′R″ group, with R′ and R″ as described herein.

The term “heterocyclyl” describes a monocyclic or fused ring group having in the ring(s) one or more atoms such as nitrogen, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” or “carboxylate” describes a —C(O)OR′ group, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(0)2R′ group, where R′ is as defined herein.

A “carbamyl” or “carbamate” group describes an —OC(O)NR′R″ group, where R′ is as defined herein and R″ is as defined for R′.

A “nitro” group refers to a —NO2 group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)—linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N—linking group, as these phrases are defined hereinabove, with R′ as defined hereinabove.

The term “guanidine” describes a —R′NC(N)NR″R′″ end group or a —R′NC(N) NR″—linking group, as these phrases are defined hereinabove, where R′, R″ and R′″ are as defined herein.

As used herein, the term “azide” refers to a —N3 group.

The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove.

The term “phosphinyl” describes a —PR′R″ group, with R′ and R″ as defined hereinabove.

The term “alkylaryl” describes an alkyl, as defined herein, which substituted by an aryl, as described herein. An exemplary alkylaryl is benzyl.

The term “heteroaryl” describes a monocyclic (e.g. C5-C6 heteroaryl ring) or fused ring (i.e. rings which share an adjacent pair of atoms) group having in the ring(s) one or more atoms, such as, for example, nitrogen, oxygen, and sulfur and, in addition, having a completely conjugated pi-electron system. In some embodiments, the terms “heteroaryl” and “C5-C6 heteroaryl” are used herein interchangeably. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine, and the like.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

The term “haloalkyl” describes an alkyl group as defined above, further substituted by one or more halide(s).

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure, or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/s interchangeable with “consisting”.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

### EXAMPLES

Generally, the nomenclature used herein, and the laboratory procedures utilized in the present invention include molecular, biochemical, and microbiological techniques. Such techniques are thoroughly explained in the literature.

### Example 1

### Synthetic Procedures

Non-limiting exemplary synthetic procedures of the conjugates of the invention are provided hereinbelow.

A typical synthesis is shown in scheme 1 with a well-established estrone protection and subsequent LiAlH/AlCl₃ ring-opening. This crude material could be alkylated selectively on the phenol using 3-5-difluorobenzyl bromide 4. Purification and subsequent Mitsunobu to install the perfluorinated motif gave compound 5 in good yield. An hydroxymethyl was introduced through reaction with DMF and LDA to provide the intermediate aldehyde which in turn was reduced in a one-pot procedure using sodium borohydride. The alcohol was activated using mesylchloride and subsequently treated with methylaminohexanol provided alcohol 7. Presumably an intermediate workup could be performed but the presence of the chloride-salt might substitute the mesylate with the chloride to provide a much less active benzylic chloride.

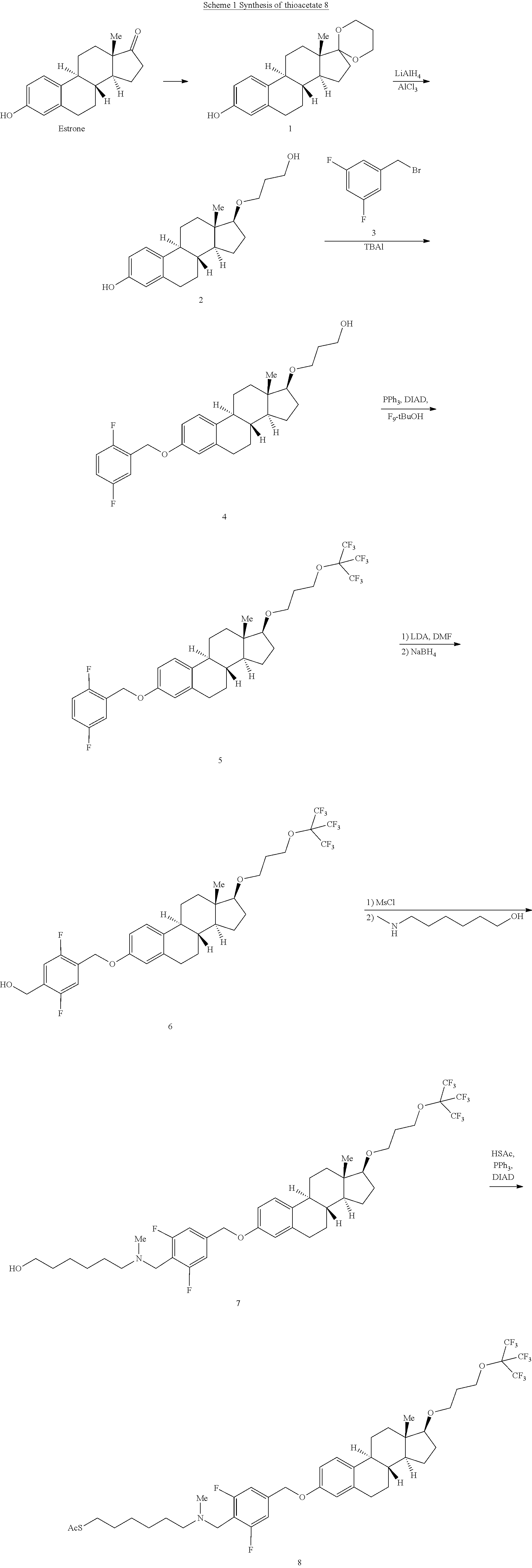

Using Mitsunobu conditions the alcohol was transformed to thioacetate 8.

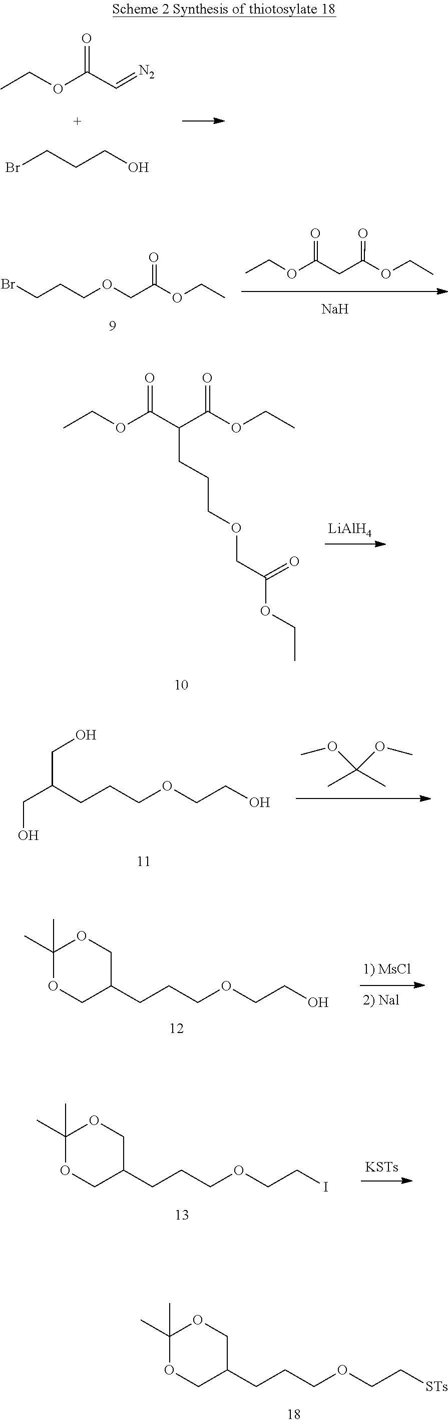

The synthesis of thiotosylate 18 started with the coupling of ethyl diazoacetate with 3-bromoprapan-1-ol to provide ether 9. Diethyl malonate was alkylated with bromide 9 to obtain triester 10. Reduction of the esters using LiAlH₄ provided triol 11. The acetonide moiety was installed by treatment with dimethoxypropane to provide acetonide 12. The alcohol was converted to first the mesylate and then using sodium iodide compound 13. Alternative methods based on triphenylphosphine and NBS generally gave rise to many impurities and low yields. Finally, the halogen was substituted with potassium thiotosylate to provide the desired building block thiotosylate 18.

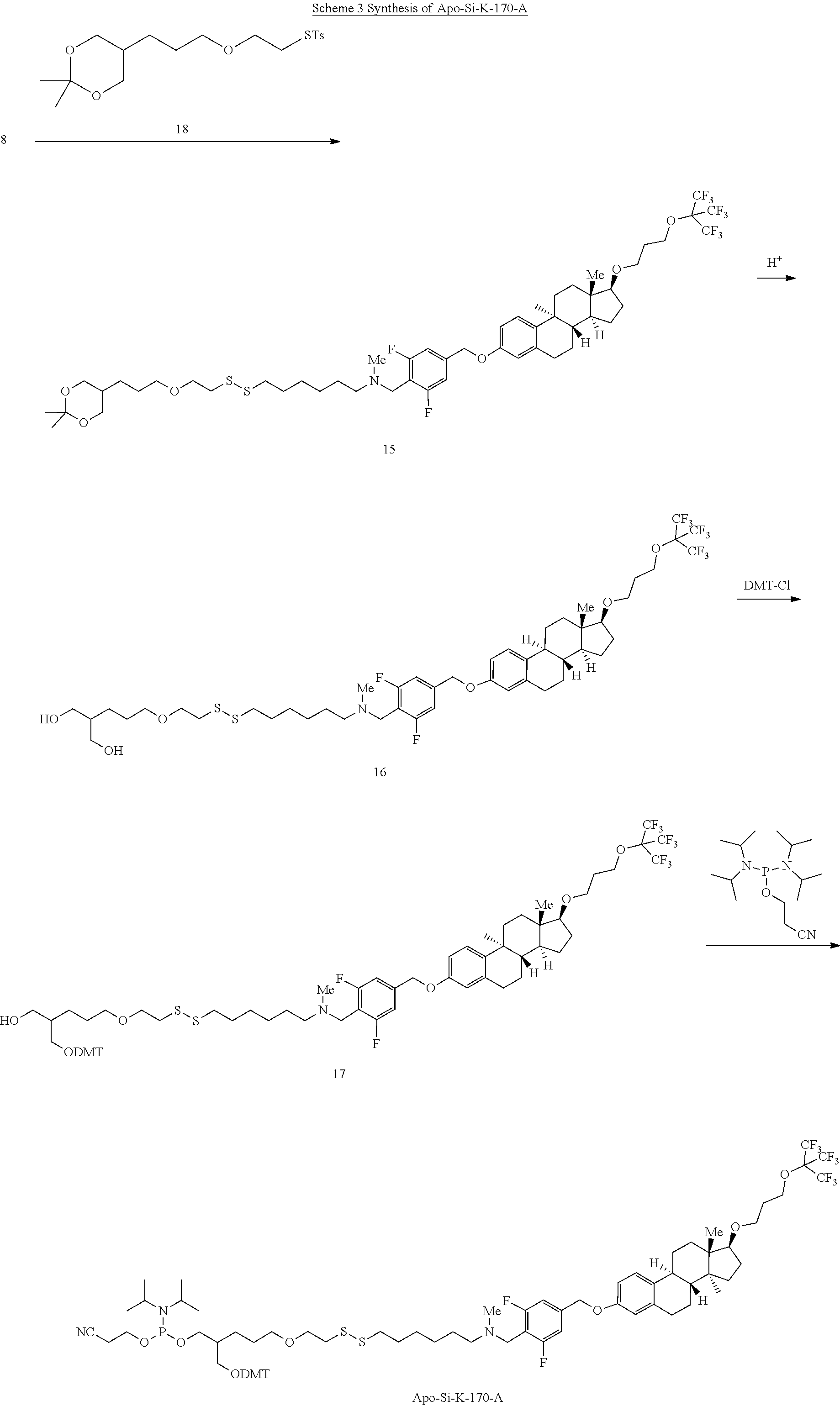

Thioacetate 8 was in-situ deprotected using basic conditions, which also allows for a nucleophilic attack on thiotosylate 18 to form disulfide 15. It should be noted that during the exposure of the acetonide on silica it might spontaneously fall off and the deprotected diol sticks to the column. Further eluting the column with up to 100% acetone could provide the diol in reasonable purity and quantity. After purification the material was submitted for the acetonide-removal using acidic protic conditions.

After crude workup the material was directly suitable for selective dimethoxytrityl mono-protection, to provide 17. The final installment of the phosphoramidate is again well-established chemistry and Apo-Si-K-170-A was obtained in good yield.

Synthesis of thioacetate 8: (8R,9S,13S,14S)-13-Methyl-6,7,8,9,11,12,13,14,15,16-decahydrospiro[cyclopenta[a]phenanthrene-17,2′-[1,3]dioxan]-3-ol (1)

To a suspension of estrone (252 gram, 0.93 mol) in toluene (1.5 L) were added trimethoxymethane (297 g, 350 mL, 2.80 mol), propane-1,3-diol (213 g, 250 mL, 2.80 mol) and pTsOH (2 g 10 mmol). The mixture was warmed to 60° C. and stirred for 16 h. Added triethylamine (6 mL) and water (600 mL) and continued stirring for 1 additional hour. Phases were separated and the organic layer was washed with water (3×400 mL) and brine. The mixture was dried over Na₂SO₄ and partially concentrated to ca 1 L. The mixture was poured into heptane (4 L) and the white solids were filtered off, washed with heptane, and dried in vacuo. Compound 1 (271 gram, 825 mmol) was isolated as a white solid in 88.5% yield.

(8R,9S,13S,14S,17S)-17-(3-Hydroxypropoxy)-13-methyl-7,8,9,11,12,13,14,15,16,17-decahydro-6H-cyclopenta[a]phenanthren-3-ol (2)

To a solution of (13S)-13-methyl-6,7,8,9,11,12,13,14,15,16-decahydrospiro[cyclopenta[a]phenanthrene-17,2′-[1,3]dioxan]-3-ol (45 g, 140 mmol) in THF (1 L) at 0° C. was added carefully lithium aluminum hydride (6.2 g, 0.16 mol), then an additional amount of THF (1.5 L) was added. While still at 0° C., the addition of aluminum chloride (73 g, 0.55 mol)(very exothermic!) was done. Stirred 15 min at 0° C., then warmed to 60° C. Stirred 2 h at 60° C. (take care with clogging), then cooled to 0° C. and started quenching dropwise with NH₄Cl (aq) (500 mL). Stirred 16h at room temperature. The phases were separated, and the organic layer was washed with brine, concentrated. A white solid was obtained, which was contaminated with estradiol (ca 15%).

This reaction was repeated in identical fashion once more using similar quantities. After NMR-analysis, both portions were combined, providing 95-gram crude material.

3-(((13S,17S)-3-((4-(2,2-dimethoxyethyl)-3,5-difluorobenzyl)oxy)-13-methyl-7,8,9,11,12,13,14,15,16,17-decahydro-6H-cyclopenta[a]phenanthren-17-yl)oxy)propan-1-ol (3)

To a suspension of crude phenol 7 (95 gram, assume 0.22 mol) potassium carbonate (76 g, 0.55 mol) in acetone (1.5 L) and MeOH (200 mL) was added 3,5-difluorobenzyl bromide (100 g, 0.49 mol) and TBAI (5.1 gram, 14 mmol). The resulting mixture was stirred for 16 hours at 65° C. The mixture was cooled to room temperature and filtered, and the filtrate was concentrated. Water (500 mL) was added, and the mixture was extracted with EtOAc (3×500 ml) and the combined organic layers were dried over sodium sulfate and concentrated.

The crude material was fully converted to the corresponding benzylic phenol.

(13S,17S)-3-((4-(2,2-dimethoxyethyl)-3,5-difluorobenzyl)oxy)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthrene (5)

To a solution of crude alcohol 8 in THF (700 mL) were added triphenylphosphine (86 g, 0.33 mol) and 1,1,1,3,3,3-hexafluoro-2-(trifluoromethyl)propan-2-ol (100 gram, 0.41 mol), followed by DIAD (59 mL, 0.3 mol) and the resulting mixture was stirred for 1 hours at room temperature. To the mixture was added heptane (0.5 L) and the mixture was partially concentrated. More heptane (0.3 L) was added, and the mixture was stirred for 5 min. The solids were filtered off and All organics were washed with aqueous 5% hydrogen peroxide (3×100 mL), twice with brine and concentrated. Further purification using column chromatography (gradient 5% to 10% EtOAc/heptane) gave compound 5 (137 gram, 0.14 mol, 74% (4 steps) as white crystalline solid.

(2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)phenyl)methanol (6)

To a solution of (13S,17S)-3-((3,5-difluorobenzyl)oxy)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthrene (73 g, 0.11 mol) in THF (1.3 L anhyd) was added at −78° C. nBuLi (45 mL, 2.5 molar, 0.11 mol). A dark color was observed, cleared up after 10 min. The resulting yellow/red mixture was stirred at for 1.5 hours at −78° C. DMF (25 mL, 0.32 mol) (anhydrous) was added dropwise and stirred for 30 minutes while warming to room temperature. Added MeOH (100 mL) and added sodium borohydride (6.1 g, 0.16 mol)(carefully) and continued stirring for 0.5. Added water (100 mL) and stirred continued for 16 h, then partially concentrated in vacuo. Added EtOAc (1 L) and washed with brine and concentrated. Further purification using flash chromatography (large column, 15% to 25% EtOAc in heptane) provided the desired alcohol (23.6 gram, 33.5 mmol) in 31% yield as a clear oil. The remainder was unreacted starting material.

6-((2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexan-1-ol (7)

To a solution of compound 10 (23.6 g, 33.5 mmol) and triethylamine (14.0 mL, 100 mmol) in DCM (250 mL) was added mesyl-C1 (3.0 mL, 38.5 mmol) and the resulting mixture was stirred for 1 hour. To the solution was added 6-(methylamino)hexan-1-ol (9 g, 68.5 mmol) were added. The resulting mixture was stirred at room temperature for 16 hours.

The mixture was diluted with dichloromethane (200 mL) and the mixture was washed with aqueous saturated sodium bicarbonate and brine, dried over Na₂SO₄ and concentrated. The crude material was purified using column chromatography (10-20% acetone in heptane +1% NEt₃) to provide alcohol 7 (15.5 g, 59.1%) as a white solid.

S-(6-((2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl) ethanethioate (8)

Two batches were setup but eventually combined prior to purification.

To a solution of alcohol 7 (23.9 g, 29.2 mmol) (15.5 g, 19.0 mmol) in THF (800 mL) were added triphenylphosphine (10.7 g, 40.9 mmol) (7.0 g, 26.5 mmol) and DIAD (7.4 mL, 38 mmol) (4.8 mL, 24.6 mmol) and the mixture was stirred for 5 minutes. Then, thioacetic acid (5.3 mL, 38.0 mmol) (3.4 mL, 47.4 mmol) was added and the mixture was stirred for 2 h. The mixtures were combined and concentrated. The crude material was purified using column chromatography (gradient 5% to 10% acetone in heptane+1% Et₃N) to provide thioacetate 8 (39.3 g, 93%).

Synthesis of Thiotosylate 18: Ethyl 2-(3-bromopropoxy)acetate (9)

To a solution of ethyl 2-diazoacetate (100 g, 0.74 mol) and 3-bromopropan-1-ol (0.10 kg, 74 mL, 0.74 mol) in DCM (100 mL) was added at 0° C. BF₃·OEt₂ (1.1 g, 0.94 mL, 7.4 mmol) was added. The reaction was stirred at 0° C. for 15 min and at room temperature for 3 hours until no more gas development was observed. The mixture was diluted with DCM (500 mL) and the mixture was washed with H₂O (500 mL) and brine (500 mL) and dried over Na₂SO₄. The solvents were removed in vacuo to provide ethyl 2-(3-bromopropoxy)acetate (9, 180 g, 0.80 mol, 110%) as a clear yellow oil.

diethyl 2-(3-(2-ethoxy-2-oxoethoxy)propyl)malonate (10)

To an ice-cooled suspension of sodium hydride (8.9 g, 0.22 mol) in DMF (600 mL) was added diethyl malonate (53 g, 51 mL, 0.33 mol) slowly. The resulting mixture was stirred for 45 minutes at room temperature. Bromide 9 (50 g, 0.22 mol) was added at 0° C. and the mixture was stirred at 0° C. for 10 minutes and at room temperature overnight. The mixture was partially concentrated. Then, water (1 L) was added, and the mixture was extracted with EtOAc/heptane (1:1, 3×500 mL). The combined organic layers were washed with brine, dried over Na₂SO₄, and concentrated. The crude material was purified by column chromatography (20% EtOAc/heptane) to provide triester 10 (45 g, 0.15 mol, 67%) as a clear oil.

2-(3-(2,2-dimethyl-1,3-dioxan-5-yl)propoxy)ethan-1-ol (12)

To an ice-cooled suspension of LiAlH₄ (25 g, 0.66 mol) in THF (200 mL) was added a solution of triester 10 (36 g, 118 mmol) in THF slowly. The mixture was allowed to warm to room temperature and stirred for 1 hours. At 0° C. KOH (aq. 20%, 106 mL (160 mL/mol LiAlH₄)) was added slowly and the resulting mixture was stirred at room temperature for 1 h min. The mixture was filtered over celite and dried over Na₂SO₄ and concentrated to provide triol 11.

The material was dissolved in DCM (400 mL) and 2,2-dimethoxypropane (15 g, 17 mL, 141 mmol) and 4-methylbenzenesulfonic acid hydrate (2.2 g, 12 mmol) were added and the resulting mixture was stirred for 30 minutes. The mixture was concentrated partially. The residue was dissolved in EtOAc (550 mL) and was washed with NaHCO₃ (300 mL) and brine (300 mL), dried over Na₂SO₄ and concentrated to provide acetonide 12 (8.9 g, 41 mmol, 35%) as a clear yellow oil.

5-(3-(2-bromoethoxy)propyl)-2,2-dimethyl-1,3-dioxane (18)

To a solution of alcohol 16 (32 g, 147 mmol) and triethylamine (30 mL, 220 mmol) in DCM (120 mL) was added MsCl (13.7 mL, 176 mmol) and the resulting mixture was stirred at room temperature for 30 min. The mixture was washed with NaHCO₃ (300 mL), dried over Na₂SO₄, and concentrated. The crude intermediate was taken up in acetone (600 mL) and sodium iodide (43.9 gram, 293 mmol) was added. The resulting mixture was refluxed for 16 h. The mixture was cooled to room temperature and concentrated. The mixture was diluted with dichloromethane and washed with water to remove all salts. The organic layer was dried over sodium sulfate and concentrated. All material was dissolved in acetone (600 ml) and potassium 4-methylbenzenesulfonothioate (49.8 g, 220 mmol) and the resulting mixture was stirred for 16 h at 60° C. The mixture was cooled to room temperature, concentrated and diluted with EtOAc and washed with aqueous saturated sodium bicarbonate, brine, dried over sodium sulfate and concentrated.

### Synthesis of Apo-Si-K-170-A

N-(2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)-6-((2-(3-(2,2-dimethyl-1,3-dioxan-5-yl)propoxy)ethyl)disulfaneyl)-N-methylhexan-1-amine (15)

This reaction was performed in two portions:

To a solution of thioacetate 8 (19.6, 1 Eq, 22.6 mmol) and thiotosylate 18 (9.2 g, 1.5 Eq, 29.4 mmol) in DCM (500 mL) and MeOH (25 mL) was added 5.4 M of sodium methoxide in MeOH (10.5 mL, 2 Eq, 56.5 mmol) and the resulting mixture was stirred at room temperature for 1 hours. The mixture was diluted with DCM (200 mL), washed with NaHCO₃ and brine, dried over Na₂SO₄, and concentrated.

The two portions were combined and further purified by column (gradient 10-40% EtOAc/heptane+1% NEt₃) to provide disulfide 15 (29.6 g, 61%) as a yellowish oil.

The much more polar fractions contained the material without the acetal as a diol 16 (11.2 gram, 10.9 mmol, 24%).

2-(3-(2-((6-((2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl)disulfaneyl)ethoxy)propyl)propane-1,3-diol (16)

To a solution of disulfide 15 (21.2 g, 19.9 mmol) in DCM (40 mL) and MeOH (100 ml) was added p-TosOH (4.16 g, 1.2 Eq, 21.9 mmol) and the resulting mixture was stirred for 16 hours at room temperature. The reaction was quenched by addition of NEt₃ (10 mL) and the mixture was washed with aqueous saturated sodium bicarbonate, dried over sodium sulfate and concentrated to provide diol 16 (18.0 g, 88%) as an off-white solid.

2-((bis(4-methoxyphenyl)(phenyl)methoxy)methyl)-5-(2-((6-((2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl)disulfaneyl)ethoxy)pentan-1-ol (17)

To a solution diol 16 (18.0 g, 18.0 mmol) and DMAP (0.21 g, 1.8 mmol) and triethylamine (3.2 mL, 23 mmol) in DCM (350 mL) was added 4,4′-(chloro(phenyl)methylene)bis(methoxybenzene) (5.9 g, 18.0 mmol) and the resulting mixture was stirred at room temperature for 16 hours. The mixture was washed with aqueous saturated sodium bicarbonate, dried over sodium sulfate, and concentrated. The crude material was purified using column chromatography (12% acetone in heptane+1% NEt₃, using silica deactivated by pretreatment with NEt₃) to provide alcohol 17 (15.3 g, 66%) as a yellow oil.

2-((bis(4-methoxyphenyl)(phenyl)methoxy)methyl)-5-(2-((6-((2,6-difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl)disulfaneyl)ethoxy)pentyl (2-cyanoethyl) diisopropylphosphoramidite (Apo-Si-K-170-A)

To a solution of alcohol 17 (15.2 g, 11.5 mmol) in DCM (300 mL) were added 3-((bis(diisopropylamino)phosphaneyl)oxy)propanenitrile (5.5 mL, 18.0 mmol) and a solution of N-methylmorpholine (1.75 g, 17.3 mmol) and TFA (985 mg, 8.7 mmol) (0.5 M NMM and 0.25M TFA) in DCM (34.5 mL) and the resulting mixture was stirred at room temperature for 1.5 hour. The mixture was washed with aqueous saturated sodium bicarbonate, dried over sodium sulfate and concentrated. The crude material was purified by column chromatography (10% Acetone heptane+1% NEt₃, pretreated silica with NEt₃) to provide Apo-Si-K-170-A (11.8 g 67%) as a colorless oil.

### Synthetic Scheme of Apo-Si-K-170-B:

[chemical structure]

### Synthetic Scheme of Apo-Si-K-170-C:

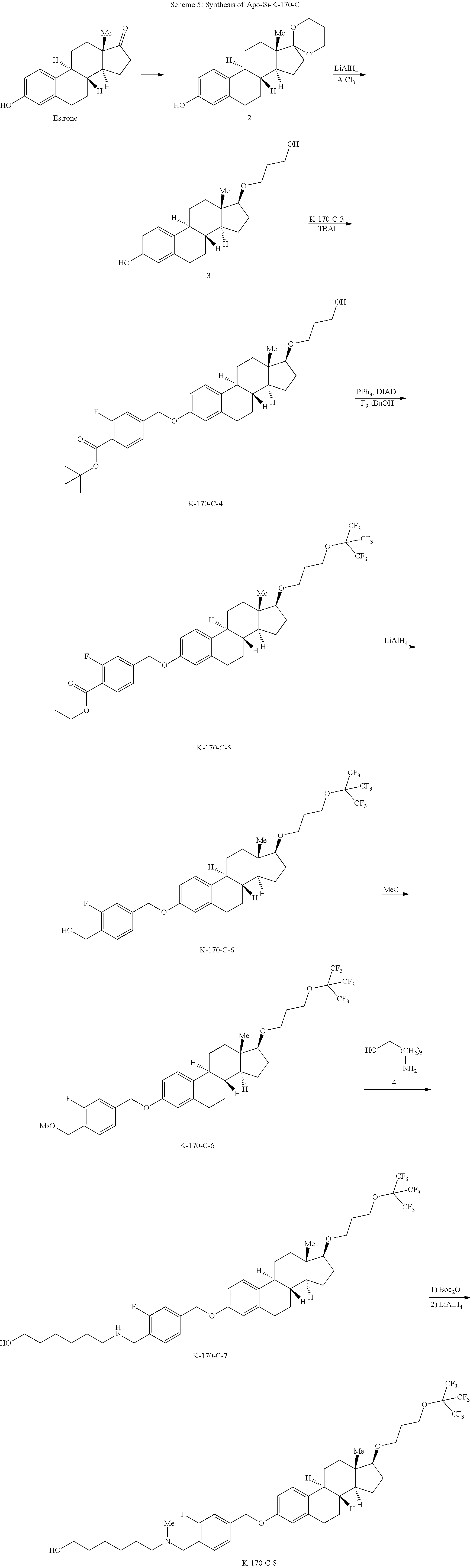

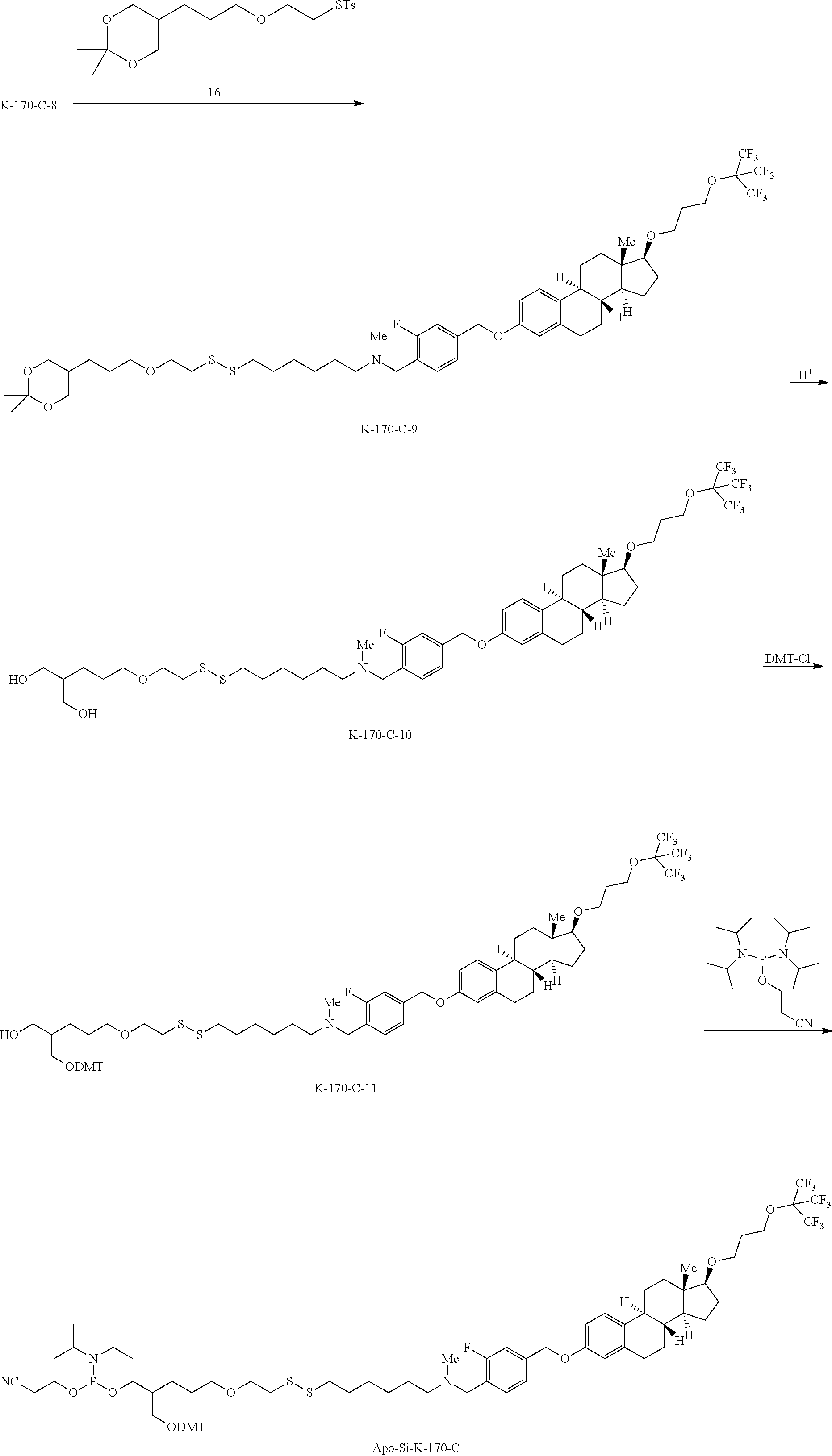

### Synthetic Scheme of Apo-Si-K-941:

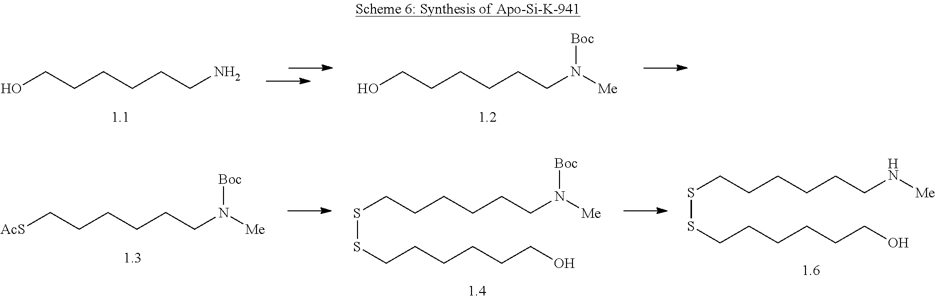

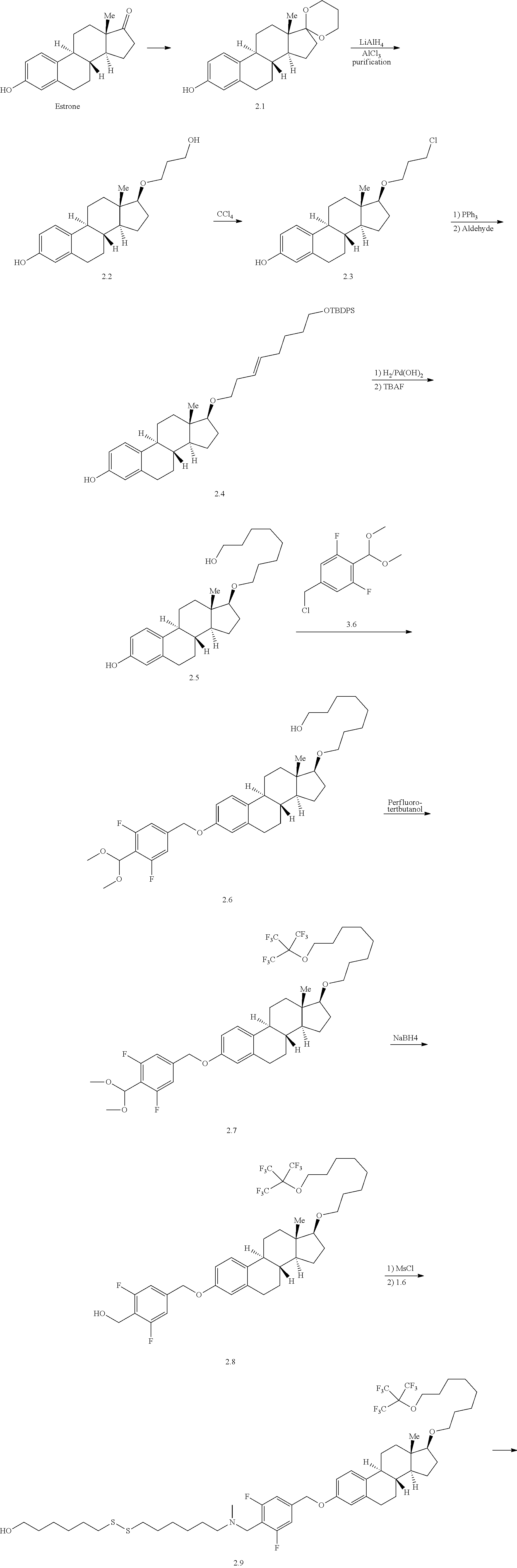

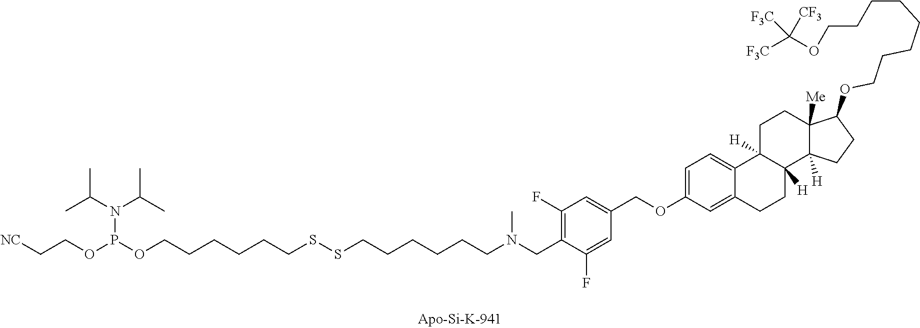 [SMILES: CC(C)N(C(C)C)P(OCCC#N)OCCCCCCSSCCCCCCN(C)Cc1c(F)cc(COc2ccc3c(c2)CC[C@@H]2[C@@H]3CC[C@]3(C)[C@@H](OCCCCCCCCOC(C(F)(F)F)(C(F)(F)F)C(F)(F)F)CC[C@@H]23)cc1F]

### Synthesis of Apo-Si-K-1014:

Initial synthetic routes toward Apo-Si-K-1014 were focused on the formation of the thiol (viz compound 4) and further functionalization. This route starts with chloride 2 which is an advanced intermediate from the Apo-Si-K-170A synthesis and can be easily converted to thioacetate 3. Also, for another Apo sense project the liberation of the thiol was required and investigated. While its liberation can be readily achieved, sulfide oxidation is a very serious concern. Upon workup solely the disulfide was isolated. Looking into the structure, the tertiary amine creates a local basic environment and sulfide oxidations are greatly enhanced in such environment. By using oxygen-free conditions for the liberation and subsequent acidification allowed a workup to obtain free thiol 4 as a HCl-salt which was found very stable. (The HCl-salt also creates a local acidic environment which might block the sulfide oxidation). Having established free thiol 4 condensations with acid 5 were found to be a logical choice but eventually such attachment was found to be very hard. Activation of the acid with DCC or Pybop failed to provide any conversion whereas an acyl chloride was found to result in very poor yields.

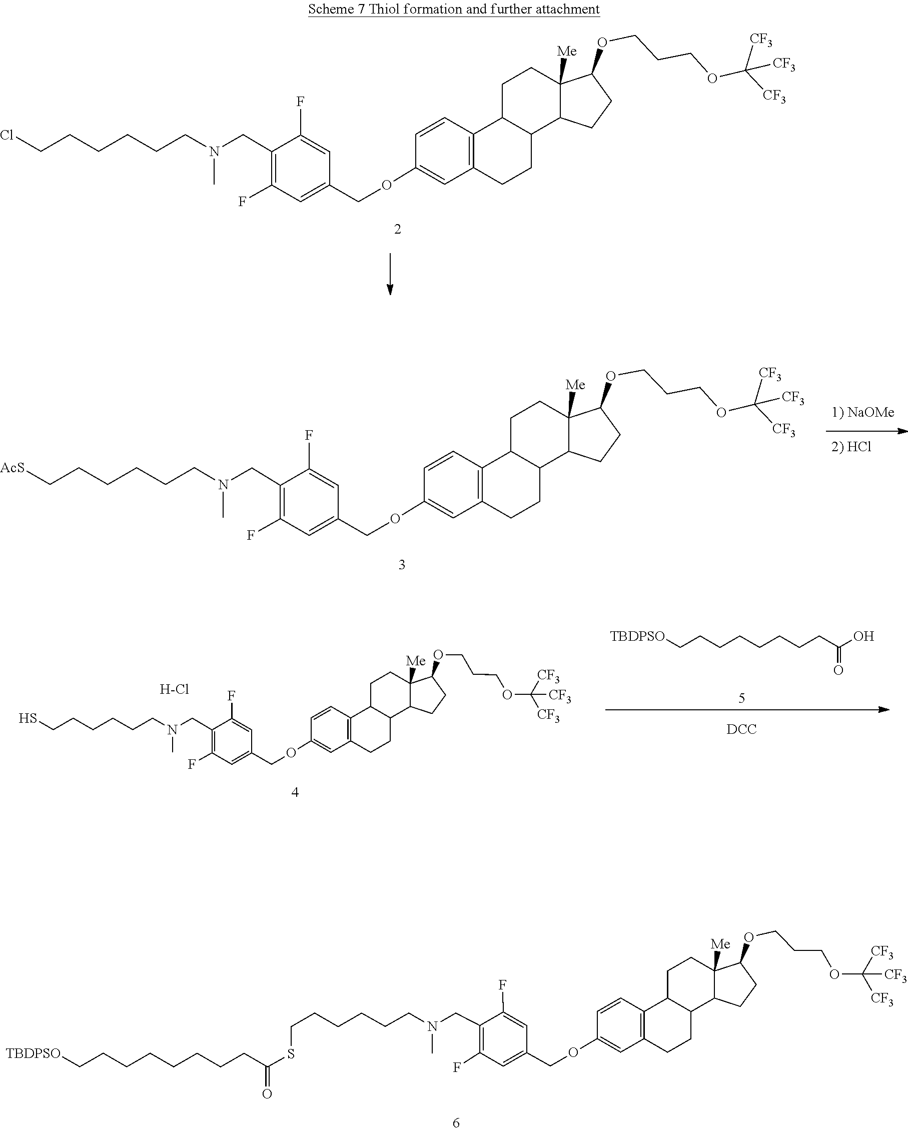

### Results and Discussion

When the thioester formation via condensation failed to provide results the notion that introduction of the thioacetate was not lost. Instead of having a laborious introduction of the thiol the attachment of a thioacid (viz compound 7) should shorten the route and provide the thioester, as shown in scheme 3.

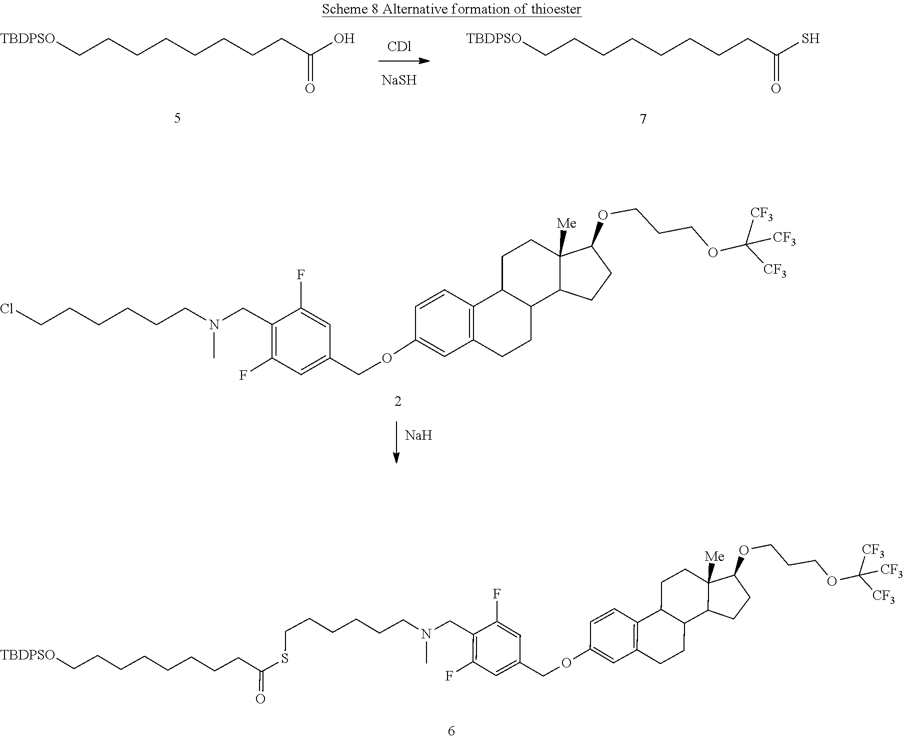

Conversion of compound 5 to the corresponding thioacid 7 was achieved by treatment with CDI and subsequently with NaSH. After acidic workup compound 7 could be isolated in almost quantitative yield and reasonable purity (a lower purity as shown by ¹H-NMR might be attributed to resonance-structures). Treating the crude material with sodium hydride was expected to form the sodium salt which in turn could be reacted with chloride 2 (this was pretreated with sodium hydride to remove the HCl-salt). Attachment of compound 7 to compound 2 was successfully performed and the thioester was found more stable than anticipated.

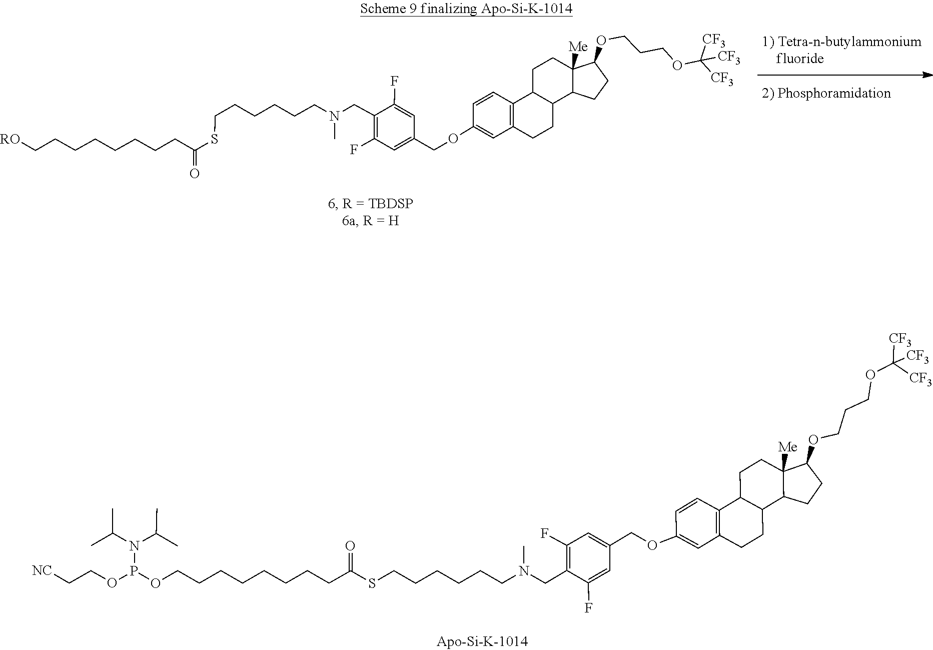

Having compound 6 in hands, treatment with TBAF liberated the alcohol and subsequent attachment of the phosphimidate using diisopropyl tetrazole. Purification provided compound Apo-Si-K-1014.

9-((tert-Butyldiphenylsilyl)oxy)nonanethioic S-acid (7). A solution of 9-((tert-butyldiphenylsilyl)oxy)nonanoic acid (2.01 g, 4.87 mmol) in dichloromethane (125 mL) at ° C. was treated with CDI (2.37 g, 14.6 mmol) and stirring continued for 1 h. Then sodium hydrogensulfide hydrate (1.08 g, 14.6 mmol) was added in one shot and stirring continued at room temperature for 16 h. The mixture was washed with 2 M HCl, dried over sodium sulfate and concentrated.

Crude NMR revealed full and relative clean conversion and the material was used as such in the next step.

S-(6-((2,6-Difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl) 9-((tert-butyldiphenylsilyl)oxy)nonanethioate (6). To a solution of compound 7 (1.95 g, 4.56 mmol) in DMF (10 mL) was added sodium hydride (326 mg, 60% Wt, 8.14 mmol). To a solution of compound 2 (2.84 g, 3.26 mmol) in DMF (1.5 mL) was added sodium hydride (163 mg, 60% Wt, 1.25 Eq, 4.07 mmol). After 5 min, both solutions were combined. Added TBAI (10 mg), warmed to 80° C. and continued for 3h. Cooled to room temperature and added heptane (350 mL) and washed with water (2×50 mL) and brine, dried over sodium sulfate and concentrated. The crude material was used as such for deprotection.

S-(6-((2,6-Difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl) 9-hydroxynonanethioate (6a) Dissolved all crude material of compound 6 in THF (75 mL) and added a 1M solution of TBAF (2.55 g, 9.77 mL, 1 molar, 9.77 mmol) in THF and continued stirring for 16 h. The mixture was concentrated and further purified using gradient flash chromatography (25% to 40% EtOAc in heptane+1% Et₃N). Compound 6a (925 mg, 0.75 mmol) was isolated as a sticky oil in 23% yield, (traces of TBDPS are still present).

S-(6-((2,6-Difluoro-4-((((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)methyl)benzyl)(methyl)amino)hexyl) 9-(((2-cyanoethoxy) (diisopropylamino)phosphaneyl)oxy)nonanethioate (Apo-Si-K-1014) To a solution of compound 6a (925 mg, 0.94 mmol) in dichloromethane (50 mL) were added diisopropyl ammonium Tetrazolide (241 mg, 1.5 1.41 mmol) and 3-((bis(diisopropylamino)phosphaneyl)oxy)propanenitrile (849 mg, 2.82 mmol). Continued stirring for 16 h at room temperature. The mixture was concentrated TLC and further purified using flash chromatography (20% to 40% ethyl acetate in heptane (very slow gradient (+1% Et₃N in all))). Apo-Si-K-1014 (825 mg, 0.69 mmol) was isolated as a sticky oil in 73% yield.

### The Synthesis of Apo-Si-K-1007

The starting point would be the usage of phenol 1 which was synthesized several times. For the sake of clarity its synthesis is included in scheme 1. First benzylation on the phenol of estradiol was performed upon which the C-17 alcohol was further functionalized with allyl bromide to provide compound 4. The olefin was hydroborated and upon oxidative workup alcohol 5 was isolated. Using Mitsunobo-conditions the alcohol was perfluorinated to provide the very apolar compound 6. The material could be readily purified by filtration over silica with heptane and further purified by recrystallization from acetonitrile. After hydrogenation and filtration phenol 1 could be obtained in significant quantity.

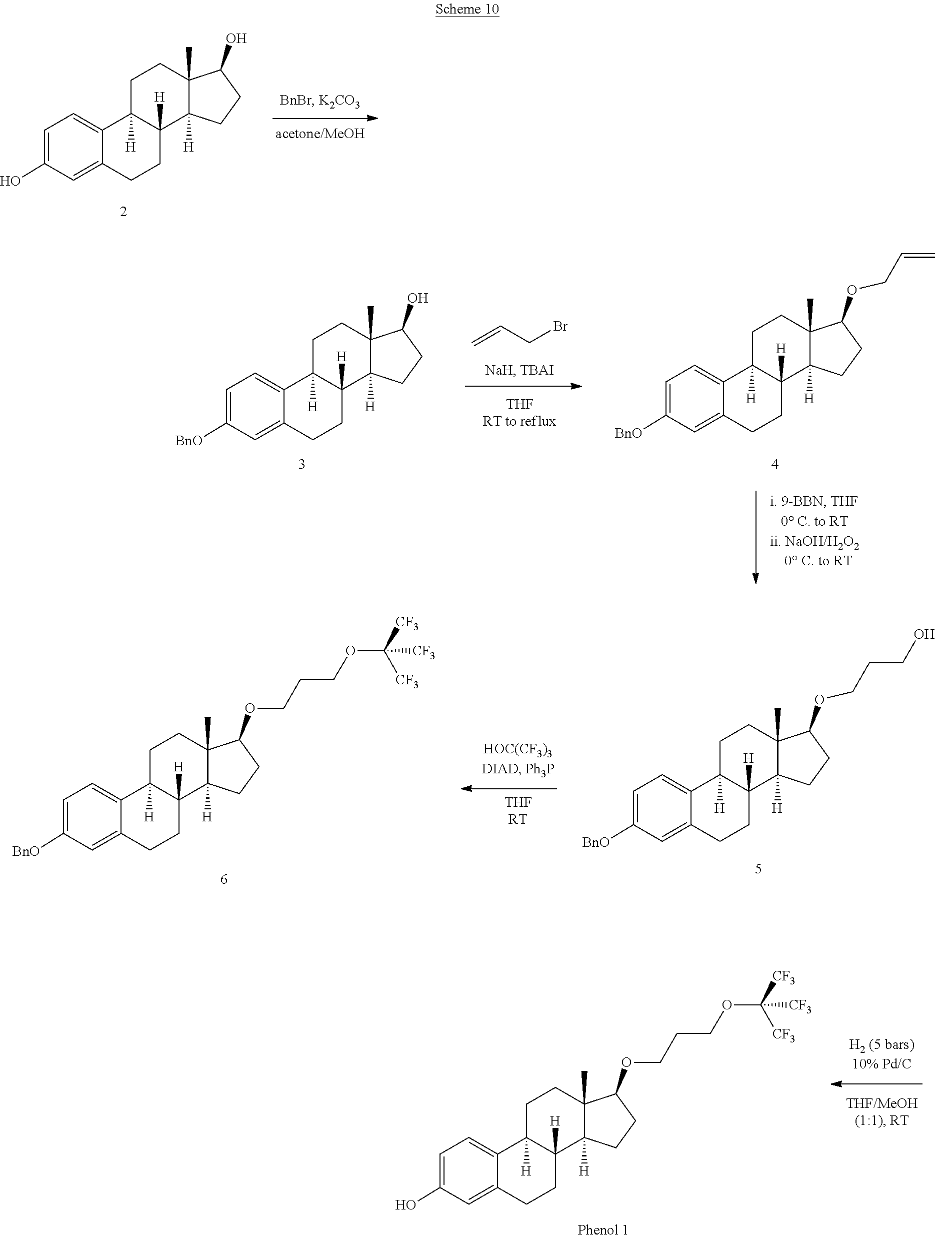

Functionalization of the free phenol can be readily performed using Mitsunobu-conditions and a suitable alcohol. We synthesized compound 7 by combining bromide 10 with functionalized piperazine 11 which resulted in compound 7. When the Mitsunobu-conditions were applied, compound 9 could be readily obtained. While Mitsunobu-reactions with phenol 1 are readily performed the piperazine motive of compound 7 somehow gave more difficulties to achieve full (and relative clean) conversion.

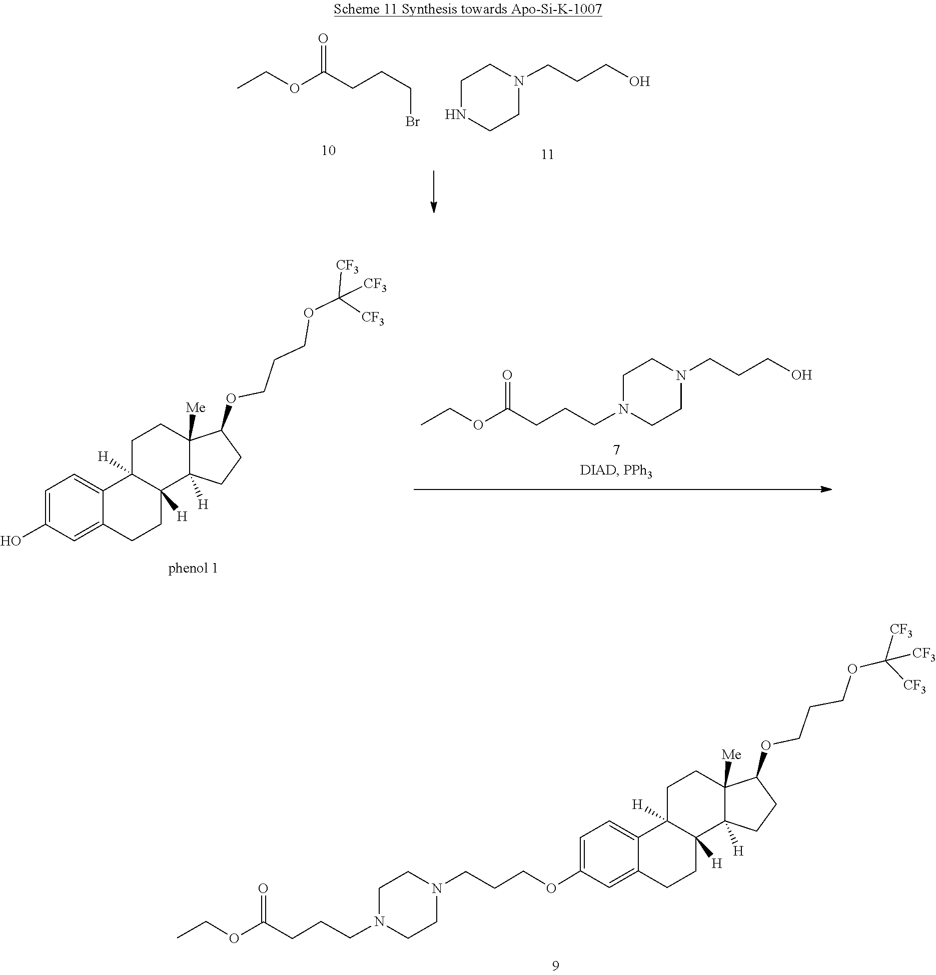

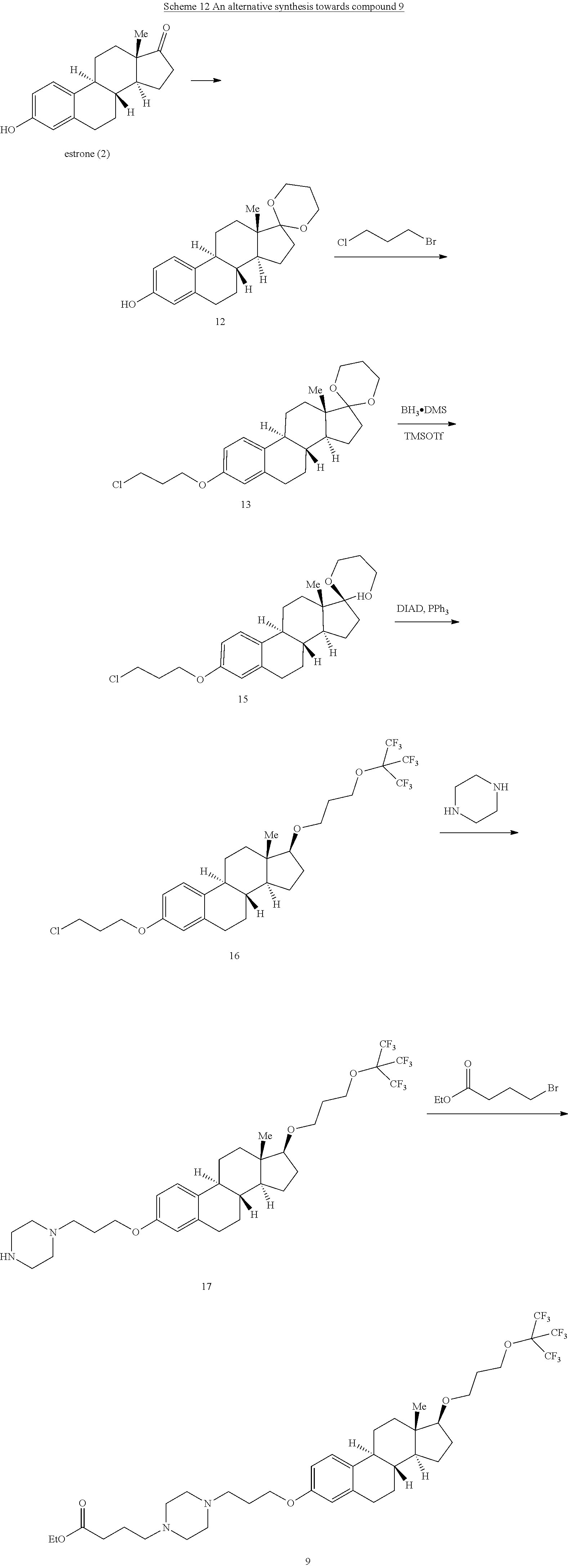

Having compound 9 in hands, liberation of the ester would provide a Zwitter-ionic species (viz compound 12) which is virtually impossible to handle. Initially we attempted to have a tert-butyl ester which could be liberated using acidic conditions such as 4 M HCl in dioxane but due to the very basic piperazine-motive the ester seems to be shielded, preventing its liberation. Therefor an ethyl ester was employed instead which could be hydrolyzed using sodium hydroxide and concentrated as such. That the handling of compound 12 and its derivatives is very difficult can be considered an understatement.

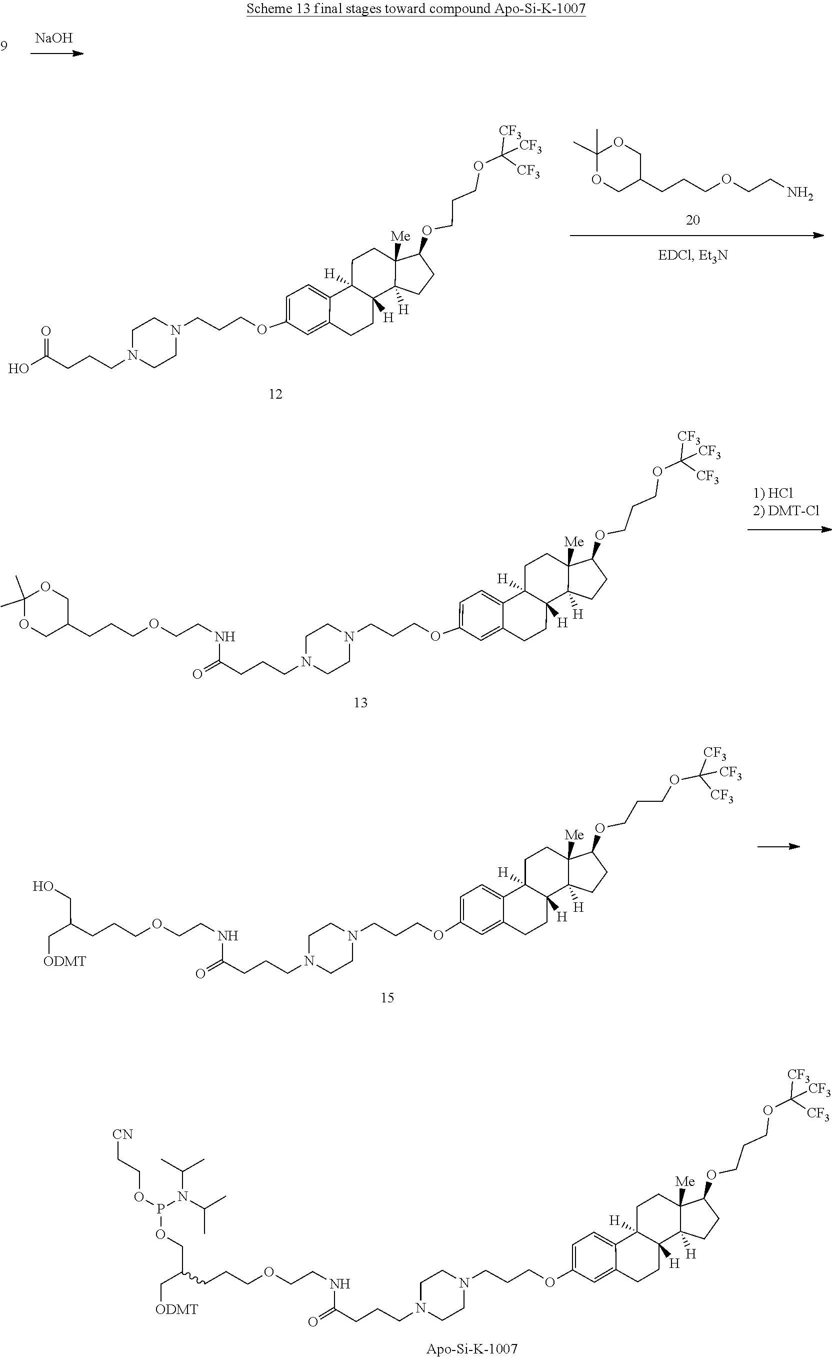

The sodium salt of compound 12 could be used for subsequent peptide couplings. We have managed to convert the material to its corresponding acyl chloride but also activation using PyBOP and EDCI were successfully performed, the latter being easier to purify. Having compound 13 in hands, removal of the acetyl-clip in an acidic medium went accordingly but provided again a very polar and difficult to handle diol. Mono-protection of the crude diol with DMT gave a more convenient to handle compound 15.

At this stage the polarity issues as well as the very basic piperazine-motive seemed responsible for all issues. The cyclic bisamine might have some sort of intramolecular interaction with the amide and somewhat Zwitterionic behavior is suspected. Moreover, for the final step we usually employ a buffer of N-methylmorpholine and TFA and suspect that the piperazine is sufficiently basic to trap the TFA indefinitely, thus making purification troublesome. The omission of a protic source however failed to activate the phosphimidate-agent which in turn allows attachment on the alcohol. To solve all these issues, the usage of a tetrazole diisopropyl salt was tested and this provided Apo-Si-K-1007 in somewhat moderate yield but good purity.

### Intermediate 20

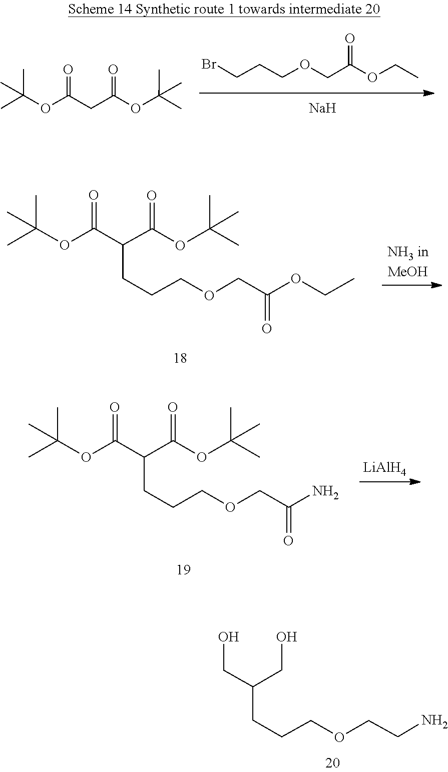

Di-tert-butyl malonate was alkylated with ethyl 2-(3-bromopropoxy)acetate and NaH in DMF at room temperature for 16 hours. Compound 18 was isolated in 59% yield and with a purity of 88.9% after purification by distillation. Subsequent amide formation was performed with 7 N NH₃ in Methanol at room temperature. After 16 hours the reaction was complete, and the mixture was concentrated. Compound 19 was isolated in almost quantitative yield and with a purity of 85.3% by GC-MS.

Reduction of compound 2 towards compound 20 proved to be challenging. First, the reduction was tested with LiAlH₄ (4.5 equiv.) in tetrahydrofuran at room temperature whereas incomplete consumption of the starting material was observed. The temperature was increased to reflux which resulted in complete consumption of the starting material. In addition, the amide was reduced to the amine while the tert-butyl esters remained unaffected. Increasing the amount of LiAlH₄ (10 equiv.) did not resolve the problems.

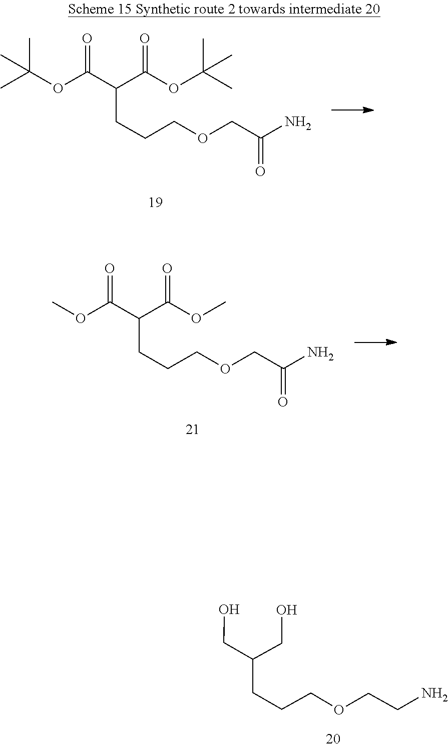 [SMILES: CC(C)(C)OC(=O)C(CCCOCC(N)=O)C(=O)OC(C)(C)C ; COC(=O)C(CCCOCC(N)=O)C(=O)OC ; NCCOCCCC(CO)CO]

Next, the transesterification was tested with 0.5 N HCl in Methanol (10 equiv.) at room temperature for 16 hours which resulted in a mixture of starting material 19 and methyl ester 21. The reaction was repeated with 2 N HCl in Methanol (10 equiv.) and after stirring for 2 days at room temperature, solely methyl ester 21 was isolated in quantitative yield. Reduction of 21 with LiAlH₄ (>7 equiv.) in tetrahydrofuran at reflux or 2-Metetrahydrofuran at room temperature did not result in de formation of 20. This was confirmed when the Boc-protection failed, and no reaction occurred. Then, the reduction was tested with borane-tetrahydrofuran (9.21 equiv.) at reflux which showed complete consumption of the starting material and no formation of product. The same applied when borane dimethyl sulfide complex (10 equiv.) was used.

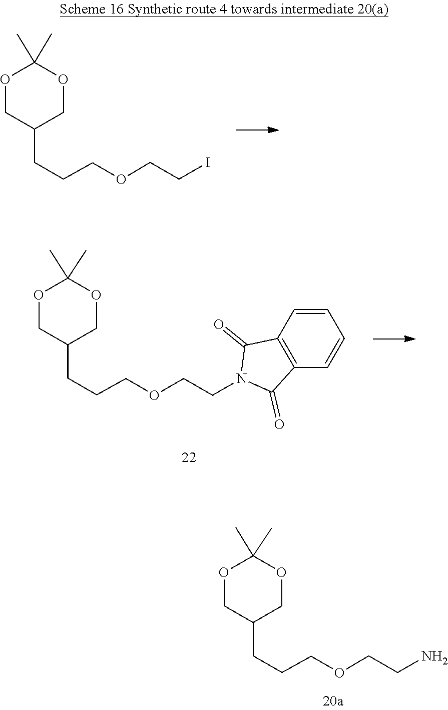 [SMILES: CC1(C)OCC(CCCOCCI)CO1 ; CC1(C)OCC(CCCOCCN2C(=O)c3ccccc3C2=O)CO1 ; CC1(C)OCC(CCCOCCN)CO1]

In the meantime, other routes were explored to furnish amine 20a. Phthalimide synthesis of the iodo starting material was successfully performed with potassium phthalimide (1.5 equiv.) in DMF at 50° C. for 8 hours and compound 22 was isolated in quantitative yield. Deprotection with hydrazine hydrate in Ethanol at reflux was complete after 16 hours. After purification by column chromatography, amine 20a was isolated in 91% yield as a mixture. It was presumed that the amine was not stable and that it needed to be protected.

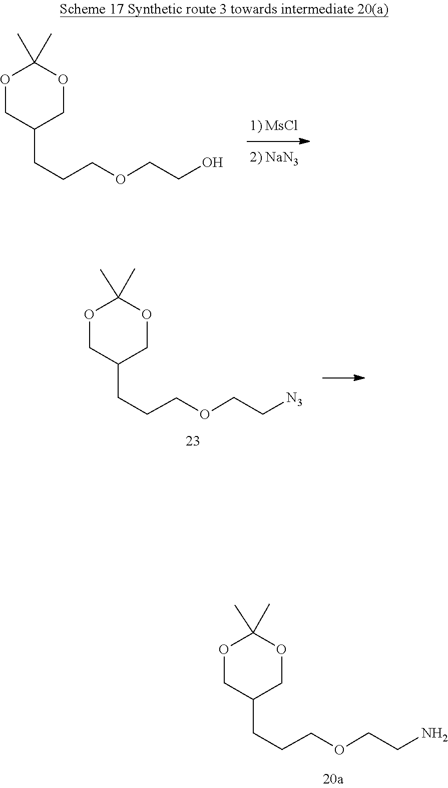 [SMILES: CC1(C)OCC(CCCOCCO)CO1 ; CC1(C)OCC(CCCOCCN=[N+]=[N-])CO1 ; CC1(C)OCC(CCCOCCN)CO1]

Having access to significant amounts of protected alcohol, an attempt to convert the material to the azide was made. The protected alcohol was reacted with Et3N (1.8 equiv.) and mesyl chloride (1.3 equiv.) in dichloromethane to its corresponding mesylate. Subsequent, reaction with sodium azide (5 equiv.) furnished azide 23 in 50% yield. The Staudinger reduction with PPh₃ in Et₂O and water at room temperature was unsuccessful and showed no conversion. While the Staudinger failed to reduce the amine, Pd/C with H₂ gave full and relative clean conversion to amine 20a.

((8R,9S,13S,14S,17S)-3-Benzyloxy-17-hydroxyestra-1,3,5(10)-triene (3)

A mixture of estradiol (2, 300 g, 1.1 mol), benzyl bromide (200 mL, 1.68 mol) and potassium carbonate (304 g, 2.2 mol) in acetone (2 L) and Methanol (0.5 L) was heated at reflux for ca 18 h. After cooling at room temperature, the reaction mixture was filtered and concentrated in vacuo. The concentrate was dissolved in hot Toluene and concentrated under reduced pressure. The crude material (508 g) was used as such in the next reaction.

(8R,9S,13S,14S,17S)-17-Allyloxy-3-benzyloxyestra-1,3,5(10)-triene (4).

Sodium hydride (110 g, 60% dispersion in mineral oil, 2.7 mol) was added portion wise to a solution of the crude alcohol 3 (508 g, ca 1.1 mol) in anhydrous tetrahydrofuran (4 L). After ca 30 min, allyl bromide (240 mL, 2.7 mol) and tetrabutylammonium iodide (40 g, 108 mmol) were added and the resulting mixture was heated at reflux for ca 18 hours. The reaction mixture was allowed to cool to room temperature and carefully quenched with water (1 L) the mixture was partially concentrated. The mixture was dissolved in Ethyl acetate (1.5 L) and washed with water (3×500 mL). The organic phase was washed with brine, dried over sodium sulfate and concentrated to afford crude compound 4 (550 g, 1.36 mol) in sufficient purity for the next step.

(8R,9S,13S,14S,17S)-3-Benzyloxy-17-(3-hydroxypropoxy)estra-1,3,5(10)-triene (5).

9-Borabicyclo[3.3.1]nonane (800 mL, 0.5 M solution in tetrahydrofuran, stabilized, 400 mmol) was added dropwise to a solution of the crude alkene 4 (101.2 g, 251 mmol) in tetrahydrofuran (1 L) at 0° C. and upon complete addition the mixture was stirred at room temperature overnight. The solution was cooled to 0° C. and slowly aqueous 30% NaOH (150 mL, 1.3 mol) and 35% aqueous (120 mL, 1.3 mol) were added dropwise simultaneously and the resulting heterogeneous mixture was vigorously stirred at room temperature for ca 1 h. The reaction mixture was then partitioned between Ethyl acetate (2 L) and brine (500 mL). The organic phase was washed with an additional 500 mL brine, dried over sodium sulfate and concentrated in vacuo. This procedure was repeated in a similar fashion and both portions were combined.

Further purification of the concentrate by flash chromatography (silica gel, gradient 25% to 35% ethyl acetate in heptanes) afforded the alcohol 5 (130 g, 310 mmol) as a white solid in 61% yield (3 steps).

(8R,9S,13S,14S,17S)-3-Benzyloxy-17-[3-(perfluoro-tert-butyloxy)propoxy]estra-1,3,5(10)-triene (6).

Diisopropyl azodicarboxylate (80 mL, 407 mmol) was added dropwise to a stirred mixture of alcohol 5 (130 g, 301 mmol), triphenylphosphine (162 g, 618 mmol), perfluoro-tert-butanol (70 mL, 497 mmol) and in dry tetrahydrofuran (2 L)under a nitrogen atmosphere. The mixture was stirred at room temperature for ca. 18 h. The reaction mixture partially concentrated and heptane (1 L) was added. After full removal of the tetrahydrofuran, precipitation started. The solids were removed using filtration and the filtrate was concentrated. Acetonitrile (1.5 L) was added, and the mixture was stirred for 30 minutes while precipitation started. The solids were collected via filtration and dried in vacuo. Compound 6 (160 g, 251 mmol) was isolated as a white solid in 81% yield.

(8R,9S,13S,14S,17S)-3-Hydroxy-17-[3-(perfluoro-tert-butyloxy)propoxy]estra-1,3,5(10)-triene (phenol 1). Notebook: MU252385-2

A Parr vessel was charged with benzyl ether 6 (160 g, 251 mmol) in Ethyl acetate (1 L) to which 10% Palladium on carbon (4 g) was added. The mixture was stirred under hydrogen pressure (5 bars) at room temperature. The reaction was monitored with ¹H NMR.

After ca 72 h, the reaction mixture was filtered through a pad of Celite (flushed with ethyl acetate) and resubmitted with fresh 10% Palladium on charcoal (4 g) to a hydrogen atmosphere (5 bars). After ca 16 h, the reaction mixture was filtered through a pad of Celite (flushed with ethyl acetate) and concentrated to provide phenol 1 (125 g, 228 mmol) as a greyish solid in 91% yield.

Ethyl 4-(4-(3-hydroxypropyl)piperazin-1-yl)butanoate (7):

Ethyl-4-bromobutyrate (11.4 g, 8.34 mL, 58.2 mmol) and 3-(1-piperazinyl)-1-propanol (8.40 g, 58.2 mmol) in acetonitrile (10 mL) was stirred 16h at room temperature. Added potassium carbonate (8.05 g, 58.2 mmol), stirred 1 h, added diethyl ether (200 mL) and filtered over a glass filter and concentrated. A clear oil isolated, used as such.

Ethyl 4-(4-(3-(((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)butanoate (9)

To a solution of (13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-ol (8.64 g, 15.8 mmol) and ethyl 4-(4-(3-hydroxypropyl)piperazin-1-yl)butanoate (5.7 g, 22 mmol) and triphenylphosphine (5.8 g, 22 mmol) in tetrahydrofuran (150 mL) was added DIAD (4.3 mL, 22 mmol). Stirred 16h at room temperature, then concentrated. The mixture was dissolved in diethyl ether (200 mL) and treated with etherous 2 M HCl (12 mL) leading to massive precipitation. The mixture was stirred for an additional 30 min, then filtered. The solids were washed with ethyl acetate. Further purification using flash (60% ethyl acetate to 60% ethyl acetate+1% Et₃N to 100% ethyl acetate+1% Et₃N in heptane) provided compound 9 (7.9 g, 10 mmol) as a clear oil in 64% yield.

4-(4-(3-(((13S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)butanoic acid (12)

To a solution of ester 9 (6.93 g, 8.79 mmol) in tetrahydrofuran (40 mL) were added sodium hydroxide (0.44 g, 11.0 mmol) and water (40 mL). Stirring continued for 18h. The seemingly oily mixture became one clear solution. The mixture was carefully concentrated (massive foaming due to its soap-like nature) in 1 L flask. Compound 12 (6.47 g, 8.5 mmol) was obtained as a solid and was used as such in the next step.

3-(3-(2,2-Dimethyl-1,3-dioxan-5-yl)propoxy)-N-(3-(4-(3-(((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propyl)propenamide.

To a solution of compound 12 (3.06 g, 3.91 mmol) and 2-(3-(2,2-dimethyl-1,3-dioxan-5-yl)propoxy)ethan-1-amine (1.19 g, 5.47 mmol) and triethylamine hydrochloride (538 mg, 3.91 mmol) in DMF (40 mL) were added DIPEA (2.1 mL, 11.7 mmol) and EDCI (900 mg, 4.69 mol). The mixture was stirred at room temperature for 72h. TLC and MS-only confirmed full attachment. To the mixture was added Ethyl acetate (50 mL) and the mixture was washed with aqueous saturated potassium carbonate, then added heptane (40 mL) and washed with water and the mixture was concentrated. Further purification using flash chromatography (2% to 10% Methanol (7 M NH₃) in dichloromethane) provided compound 13 (1.7 g 1.8 mmol) as a sticky oil.

N-(3-(4-(3-(((13S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propyl)-3-((5-hydroxy-4-(hydroxymethyl)pentyl)oxy)propanamide hydrochloride (14)

To a solution of compound 13 (1.7 g, 1 Eq, 1.8 mmol) in dichloromethane (2 ml) and methanol (75 mL) was added 37% HCl (0.5 ml). Continued stirring for 3h (massive white precipitation). The mixture was concentrated and triturated with Ethyl acetate. The white solids of HCl-salt 14 (1.7 g, 1.8 mmol) were used as such.

N-(2-((5-(bis(4-Methoxyphenyl)(phenyl)methoxy)-4-(hydroxymethyl)pentyl)oxy)ethyl)-4-(4-(3-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)butanamide (15)

Under a nitrogen atmosphere compound 14 (841 mg, 914 μmol) was dissolved in dichloromethane (100 mL). triethylamine (185 mg, 255 μL, 1.83 mmol) and 1-[chloro-(4-methoxyphenyl)-phenyl-methyl]-4-methoxy-benzene (294 mg, 868 μmol) were added and stirred at room temperature for 16h. When TLC showed almost full conversion, an additional amount of 1-[Chloro-(4-methoxyphenyl)-phenyl-methyl]-4-methoxy-benzene (31.0 mg, 91.4 μmol) was added. Stirring continued for 1 h, then the mixture was washed with aqueous saturated sodium bicarbonate, dried over sodium sulfate and concentrated. Further purification was performed using flash chromatography (gradient 5% to 7% Methanol (containing 7 M NH₃) in dichloromethane provided compound 15 (880 mg, 0.72 mmol) as a clear oil.

2-((bis(4-Methoxyphenyl)(phenyl)methoxy)methyl)-5-(2-(4-(4-(3-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)butanamido)ethoxy)pentyl (2-cyanoethyl) diisopropylphosphoramidite (Apo-Si-K-1007)

To a solution of compound 15 (880 mg, 720 μmol) in dichloromethane (50 ml) and diisopropyl ammonium tetrazolide (194 mg, 1.13 mmol) was added at 0° C. 3-((bis(diisopropylamino)phosphaneyl)oxy)propanenitrile (620 mg, 2.06 mmol). The mixture was stirred for 4 h at room temperature, then concentrated. Further purification using flash chromatography (gradient 100% ethyl acetate to 20% acetone in ethyl acetate (+1% Et₃N in all) provided Apo-Si-K-1007 (470 mg, 0.33 mmol) as a clear oil in 44% yield.

5-(3-(2-Azidoethoxy)propyl)-2,2-dimethyl-1,3-dioxane (23). To a solution of 2-(3-(2,2-dimethyl-1,3-dioxan-5-yl)propoxy)ethan-1-ol (7.0 g, 32 mmol), dichloromethane (70 mL) and triethylamine (8.0 mL, 58 mmol). The solution was cooled down to 0° C. Mesyl chloride (3.2 mL, 42 mmol) was added dropwise via a dropping funnel, while keeping the temperature below PC. The mixture was stirred at room temperature for 30 min, then quenched with aqueous saturated sodium bicarbonate, further diluted with dichloromethane and the organic layer was collected. The organic layer was dried over sodium sulfate and concentrated.

The material was dissolved in acetone (700 mL) and sodium azide (10 g, 160 mmol) and TBAI (200 mg) was added, and the material was heated to reflux. After 16 h the mixture was cooled to room temperature, filtered, and concentrated. The yellowish oil was purified using flash chromatography (30% to 60% ethyl acetate in heptane) to provide compound 23 (3.0 g 10 mmol).

5-(3-(2-Aminoethoxy)propyl)-2,2-dimethyl-1,3-dioxane name (20a): To a solution of azide 23 (4 g, 0.02 mol) in tetrahydrofuran (50 mL) and ethanol (20 mL) was added palladium on carbon (250 mg, 2.35 mmol). Vacuum was applied and the atmosphere was purged with hydrogen (3×). Stirred 16h at room temperature under a hydrogen atmosphere. Filtered over a short path of celite and concentrated. The crude material was found suitable for the next reaction.

The synthesis of key intermediate 7 is depicted in the scheme below. Having estrone protected with a propyl acetal (viz compound 3) allows for selective ring opening as means to have a C3-ether. Generally, this opening was achieved using 4 equiv AlCl₃ and 1.2 equiv LiAlH₄ and refluxing for several hours but this harsh reaction did not allow for other functional groups. Recently a milder method had been developed with TMSOTF and BH₃·DMS at −78° C. but this did not tolerate the free phenol. While typically a benzyl-protective group on the phenol suits all purposes its removal with hydrogenation is rather cumbersome. Having the desired C3-chloride (viz compound 4) in place and applying the ring-opening gave full and relative clean conversion to compound 5.

### Synthesis of Apo-Si-K-1000

The synthesis of Apo-Si-K-1000 is depicted in the schemes below:

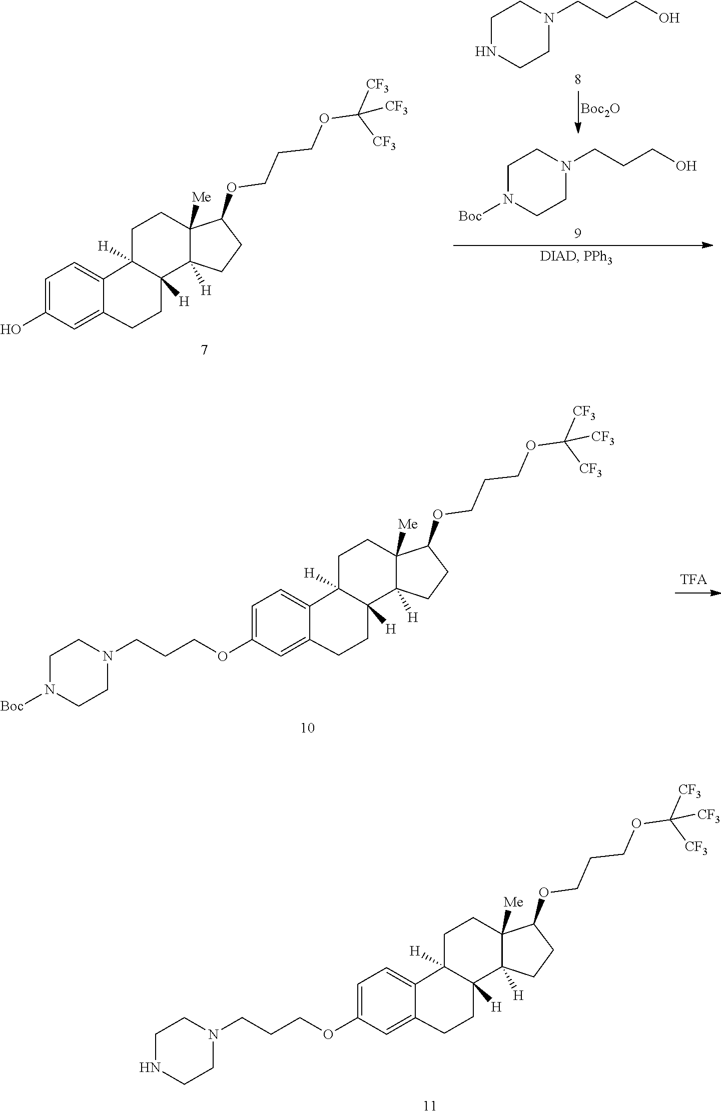 [SMILES: "Boc2O" ; OCCCN1CCNCC1 ; C[C@]12CC[C@@H]3c4ccc(O)cc4CC[C@H]3[C@@H]1CC[C@@H]2OCCCOC(C(F)(F)F)(C(F)(F)F)C(F)(F)F ; CC(C)(C)OC(=O)N1CCN(CCCO)CC1 ; CC(C)(C)OC(=O)N1CCN(CCCOc2ccc3c(c2)CC[C@@H]2[C@@H]3CC[C@]3(C)[C@@H](OCCCOC(C(F)(F)F)(C(F)(F)F)C(F)(F)F)CC[C@@H]23)CC1 ; C[C@]12CC[C@@H]3c4ccc(OCCCN5CCNCC5)cc4CC[C@H]3[C@@H]1CC[C@@H]2OCCCOC(C(F)(F)F)(C(F)(F)F)C(F)(F)F]

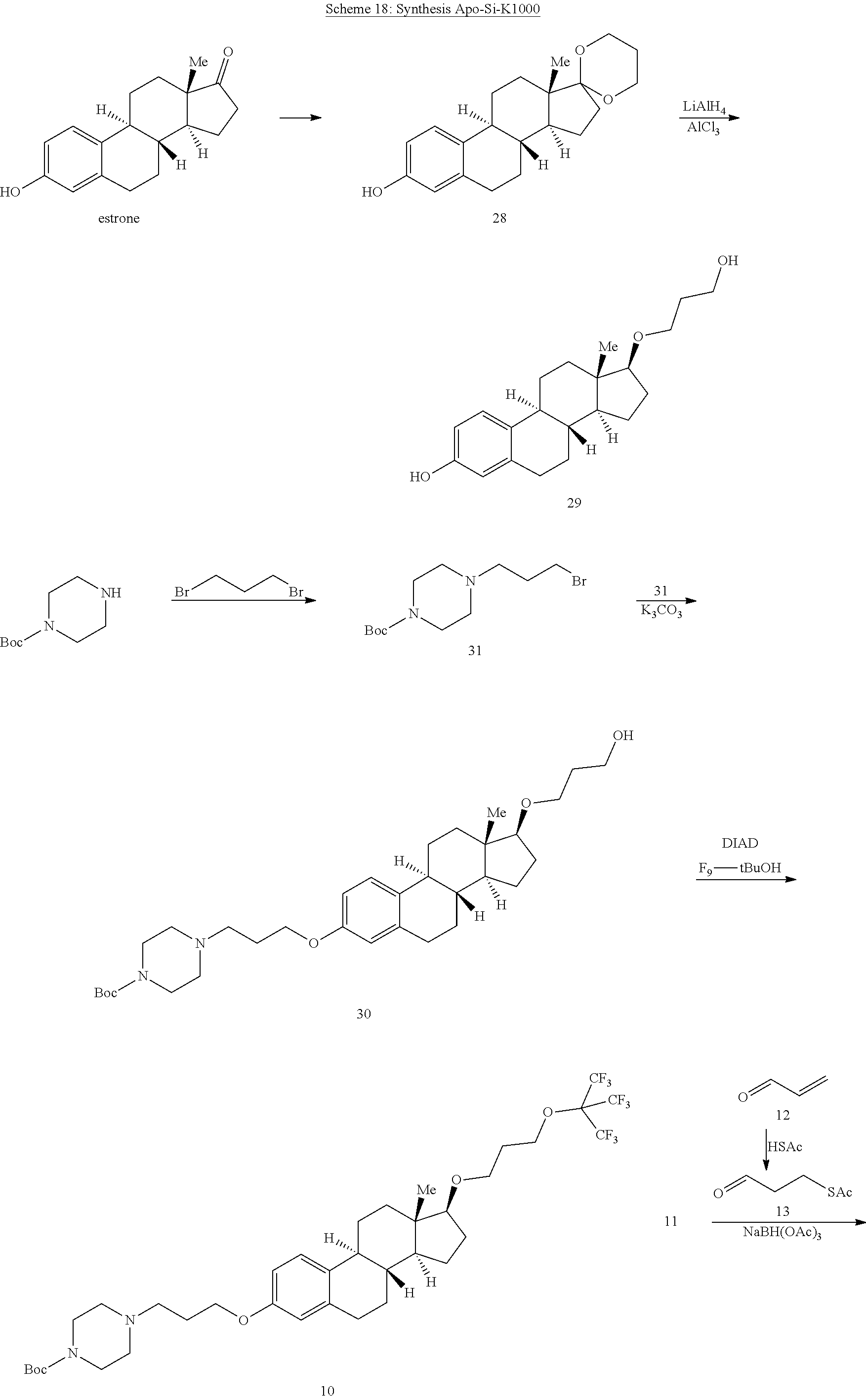

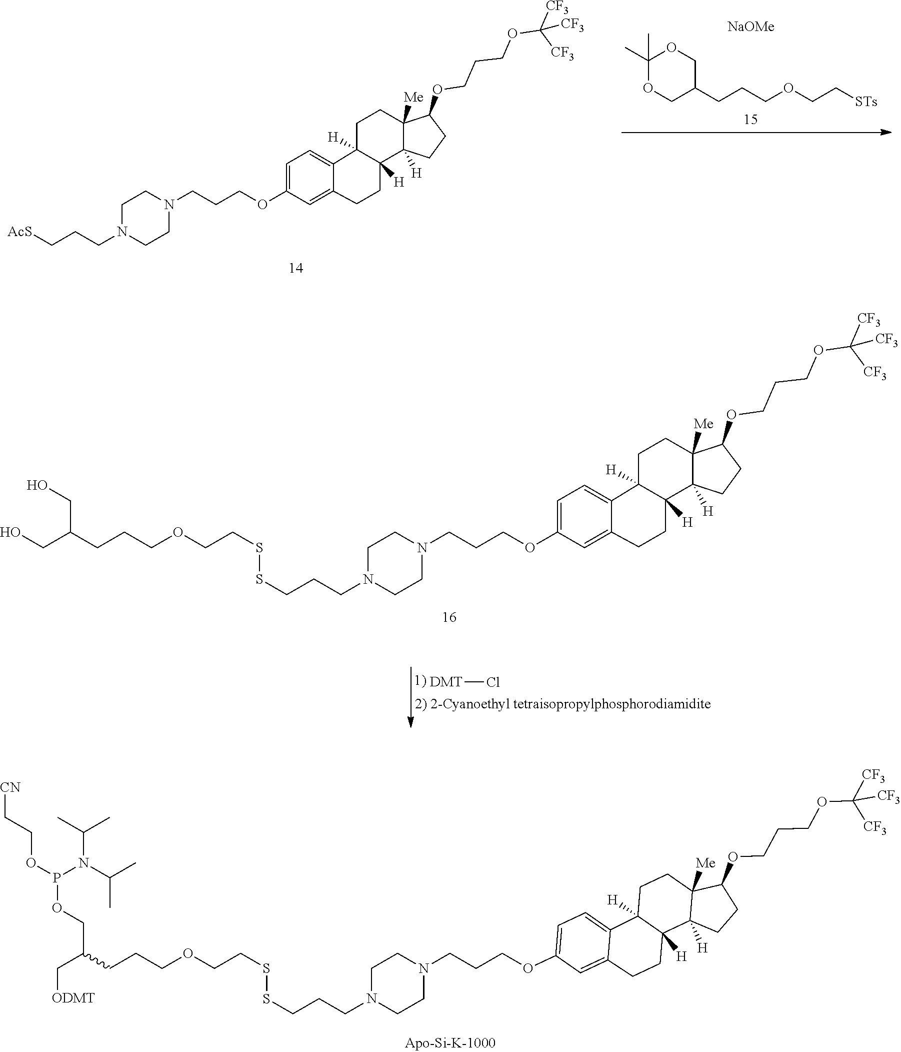

### Synthesis of Apo-Si-K-1013

The attachment of the perfluorinated motive using Mitsunobu-conditions gave rise to compound 6. For the workup dissolving all material in heptane allows the precipitation of most triphenyl phosphoxide and further purification using essentially a filtration over silica gives excellent purity since all by-products and Mitsunobu-reagents stick to silica.

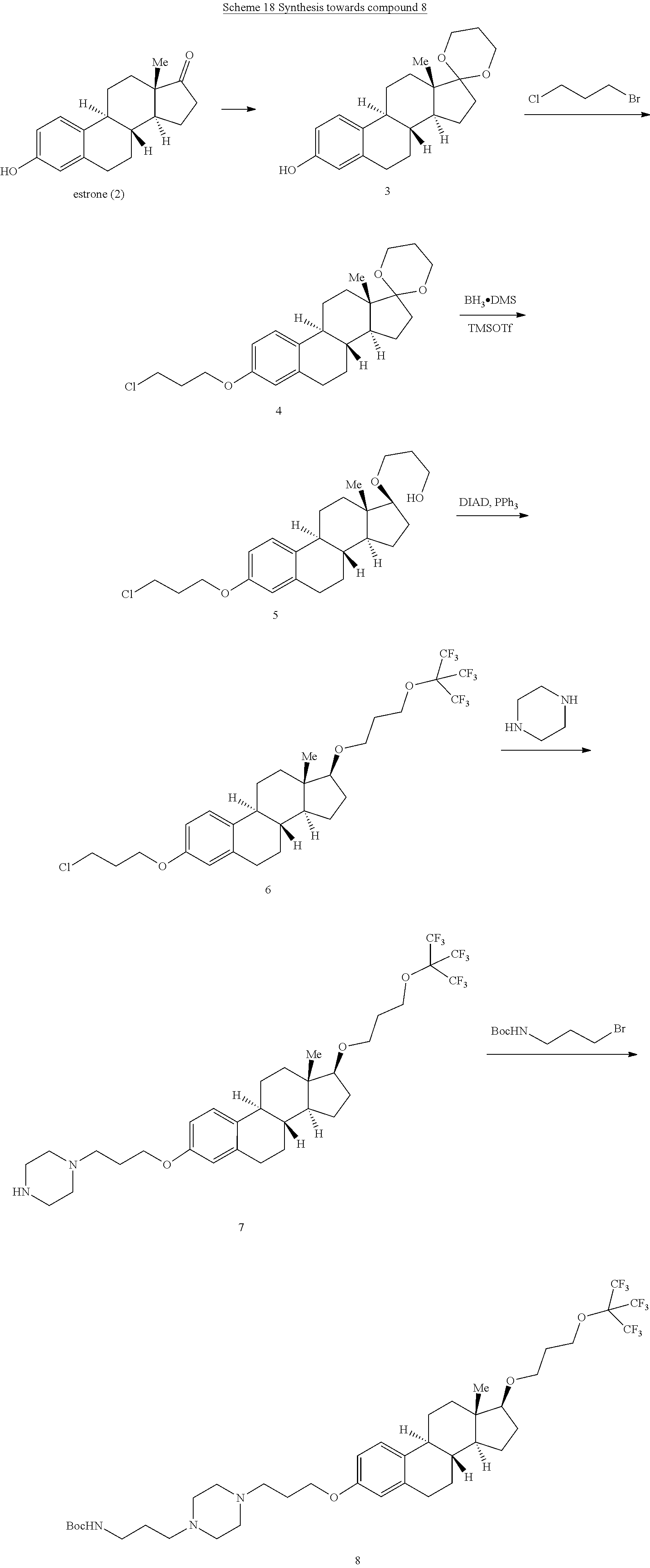

Alkylation of the piperazine on the chloride is readily performed and seems to be selective. Excess of piperazine is washed away when an aqueous workup is employed. Further functionalization to provide compound 8 can be performed by treating compound 7 with 1 equiv of the bromide.

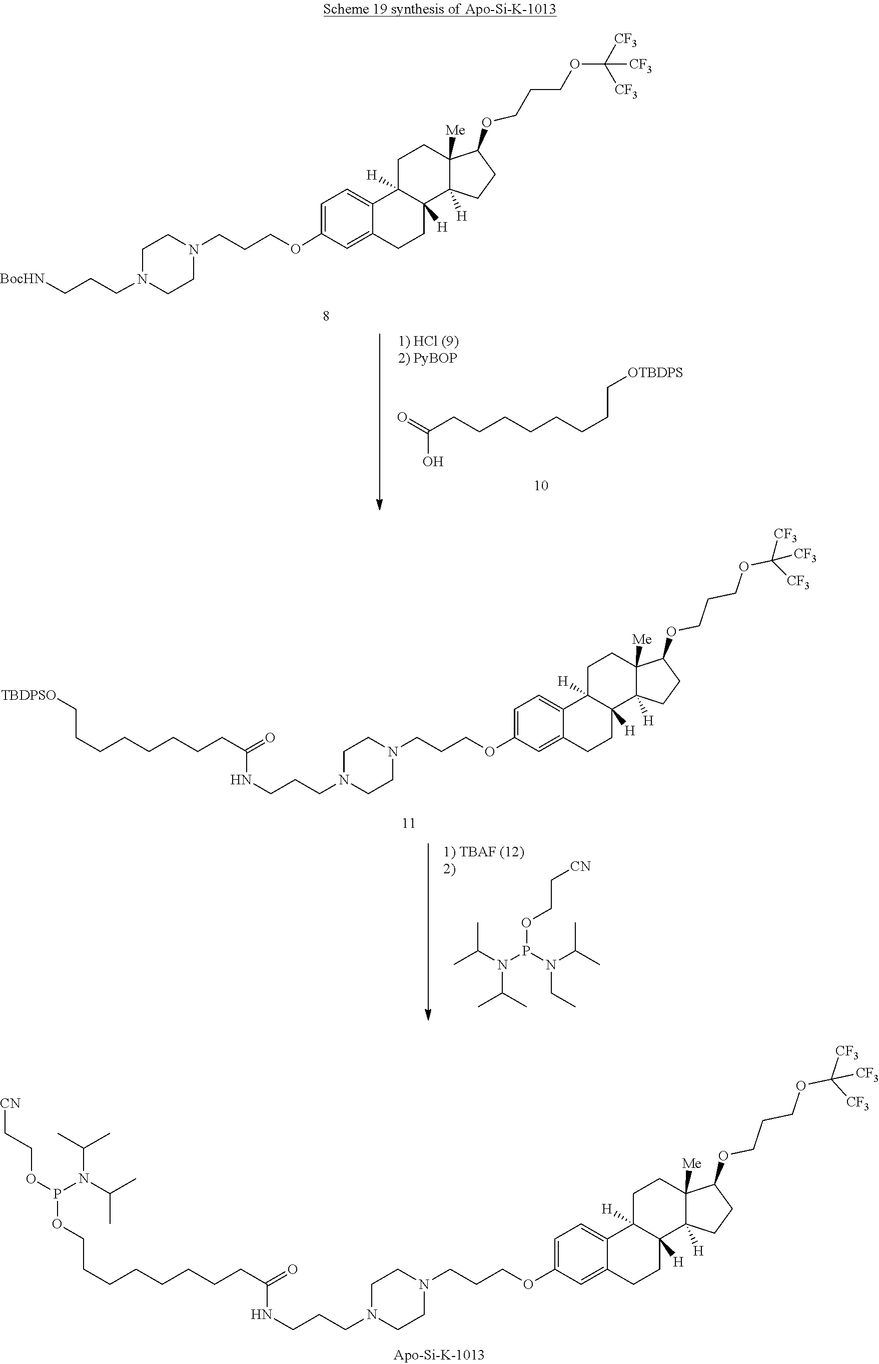

Deprotection of Boc-protected amines is always difficult in presence of bases such as the piperazine. The piperazine is first protonated causing the material to precipitate which hampers the acidic deprotection. However, using 4 M HCl in dioxane allows the material to be sufficiently soluble to allow full deprotection. Subsequent peptide coupling with either PyBOP or DCC/DMAP allows for the attachment of the acid. However, purification seems to be troublesome at this stage, presumably due to the basic motive of the piperazine and the formed amide. Treatment with TBAF and attachment of the phosphimidate were the final steps to obtain Apo-Si-K-1013.

For the final attachment we had to abandon our initial protocol (TFA, NMM) since we assume that the basic piperazine is hampering the successful removal of TFA and the final compound are difficult to obtain as salt. However, omission of TFA from the mixed failed to provide any conversion, seemingly the phosphimidate-agent requires acidic activation for successful attachment. Using diisopropylamine-tetrazole as base and Lewis acid, successful coupling was achieved whereas the Lewis acid was sufficiently weak to be removed using further purification.

(8R,9S,13S,14S)-13-Methyl-6,7,8,9,11,12,13,14,15,16-decahydrospiro[cyclopenta[a]phenanthrene-17,2′-[1,3]dioxan]-3-ol (3). Notebook: RVE21010102-01 To a suspension of (8R,9S,13S,14S)-3-hydroxy-13-methyl-6,7,8,9,11,12,13,14,15,16-decahydro-17H-cyclopenta[a]phenanthren-17-one (250 g, 1.00 Eq, 924 mmol) in Toluene (1.5 L), were added triethyl orthoformate (205 g, 231 mL, 1.5 Eq, 1.38 mol), propane-1,3-diol (105 g 100 mL, 1.5 Eq, 1.38 mol) and pTsOH (1.75 g, 0.01 Eq, 9.24 mmol). The suspension was mechanically stirred. No exotherm observed while adding the chemicals together. The mixture was warmed to 60° C. (internal) and stirred for 2 h. The mixture turned into a light-yellow solution. Reaction was stirred at 60° C. (internal) overnight. Propane-1,3-diol (35.18 g, 33.41 mL, 0.5 Eq, 462.3 mmol) and triethyl orthoformate (68.5 g, 76.9 mL, 0.5 Eq, 462 mmol) were added at 60° C. and the reaction stirred for 3 h. The reaction was cooled to 40° C., then triethylamine (4.67 g, 6.44 mL, 0.05 Eq, 46.2 mmol) and water (599 g, 599 mL, 36 Eq, 33.2 mol) were added and the mixture stirred for 10 min. Phases were separated and the organic layer was concentrated to ca 1 L and let it stand for 16h. A solid precipitated, the mixture was mechanically stirred for 4h then filtered and washed with 300 mL toluene and 200 mL heptanes, dried over suction. The product was obtained as white solid (235 g, 77.4%), grinded and the HNMR was measured.

(13S)-3-(3-Chloropropoxy)-13-methyl-6,7,8,9,11,12,13,14,15,16-decahydrospiro[cyclopenta[a]phenanthrene-17,2′-[1,3]dioxane](4).

To a solution of (13S)-13-methyl-6,7,8,9,11,12,13,14,15,16-decahydrospiro[cyclopenta[a]phenanthrene-17,2′-[1,3]dioxan]-3-ol (34.50 g, 1 Eq, 105.0 mmol) in DMF (40 mL) were added cesium carbonate (51.34 g, 1.5 Eq, 157.6 mmol) and 1-bromo-3-chloropropane (33.07 g, 20.78 mL, 2 Eq, 210.1 mmol). Continued stirring for 16 h at room temperature. The mixture was diluted in ethyl acetate (50 mL) and heptane (250 mL) and washed with water (3×50 mL) and brine (50 mL), dried over sodium sulfate and concentrated. The product was isolated as a clear oil in quantitative yield. The crude material was used as such in the next reaction.

3-(((8R,9S,13S,14S,17S)-3-(3-Chloropropoxy)-13-methyl-7,8,9,11,12,13,14,15,16,17-decahydro-6H-cyclopenta[a]phenanthren-17-yl)oxy)propan-1-ol (5).

A 2 L reactor vessel was charged with a solution of the crude material of (8R,9S,13S,14S)-3-(3-chloropropoxy)-13-methyl-6,7,8,9,11,12,13,14,15,16-decahydrospiro[cyclopenta[a]phenanthrene-17,2′-[1,3]dioxane](104 mmol) in dichloromethane (anhydrous 1 L) and the solution was cooled to −78° C. (cooled with solid dryice). Borane dimethyl sulfide complex (10.2 g, 12.8 mL, 1.3 Eq, 135 mmol) was added in one portion via a syringe (T dropped to reach −75° C. then back to −78° C.). To the mixture was added trimethylsilyl trifluoromethanesulfonate (30.0 g, 24.4 mL, 1.3 Eq, 135 mmol) in a fast stream (˜1 min) and the reaction was allowed to stir at −75° C. for 2 h. The reaction mixture as a solution was quenched with the careful addition of brine (500 mL) and stirring continued for 16 h. The layers were separated (separation clear). The organic layer was washed with water, brine and concentrated. The product was isolated as a clear oil (42.2 g, 104 mmol).

(13S,17S)-3-(4-Chlorobutoxy)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthrene (6)

3-(((13S,17S)-3-(3-chloropropoxy)-13-methyl-7,8,9,11,12,13,14,15,16,17-decahydro-6H-cyclopenta[a]phenanthren-17-yl)oxy)propan-1-ol (44 g, 1 Eq, 0.11 mol) was dissolved in THF (1 L) and cooled to 0° C. To the solution was added triphenylphosphine (40 g, 1.4 Eq, 0.15 mol), 1,1,1,3,3,3-hexafluoro-2-(trifluoromethyl)propan-2-ol (33 g, 20 mL, 1.3 Eq, 0.14 mol) and dropwise DIAD (27 g 26 mL, 97% Wt, 1.2 Eq, 0.13 mol). The reaction was stirred for 1 h at room temperature. The mixture was concentrated and treated with heptane (˜1 L) and stirred 16h, allowing a precipitation. The solids were filtered off and washed with heptane. The organics were concentrated. Further purification using flash (5% EtOAc in heptane) provided compound 6 (61.3 gram, 96 mmol) as a clear oil in 91% yield (3 steps).

1-(3-(((13S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazine (7)

A mixture of (13S,17S)-3-(3-chloropropoxy)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthrene (61.32 g, 1 Eq, 98.11 mmol), piperazinium dichloride (46.81 g, 3 Eq, 294.3 mmol) and potassium carbonate (47.46 g, 3.5 Eq, 343.4 mmol) in acetonitrile (1.5 L) was treated with 35% aqueous sodium hydroxide (34 g, 31 mL, 3 Eq, 294.3 mmol) and was refluxed for 48h. Intermediate analysis revealed partial conversion. Added piperidine (8.4 g, 98 mmol) and continued heating for an additional 16h. The mixture was cooled to room temperature, filtered and partially concentrated. Ethyl acetate (1 L) was added and the mixture was washed with water (2×150 mL) and brine, dried over sodium sulfate and concentrated. The crude material (68.0 gram) was isolated in quantitative yield and sufficiently clean for subsequent chemistry.

tert-Butyl (3-(4-(3-(((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propyl)carbamate (8).

A mixture of tert-butyl (3-bromopropyl)carbamate (1.2 g, 1.1 Eq, 5.1 mmol), 1-(3-(((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazine (3.1 g, 1 Eq, 4.6 mmol) and potassium carbonate (1.3 g, 2 Eq, 9.2 mmol) in acetonitrile (40 mL) was warmed to 60° C. for 16 h. The mixture was concentrated and further purified using flash chromatography (gradient 50% to 80% EtOAc in heptane (+1% Et₃N) Compound 8 (3.2 g, 3.8 mmol) was isolated as an oil.

3-(4-(3-(((13S,17S)-17-(3-((1,1,1,3,3,3-Hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propan-1-amine (9)

A suspension of compound 9 (3.2 g, 1 Eq, 3.8 mmol) was treated with 4 M HCl in dioxane (50 mL) and stirred for 3 h, then concentrated. The HCl-salt was obtained in quantitative yield.

9-((tert-Butyldiphenylsilyl)oxy)-N-(3-(4-(3-(((13S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propyl)nonanamide (12)

To a suspension of compound 9 (3.2 g, 1 Eq, 3.8 mmol) in dichloromethane (350 mL) were added DMAP (0.46 g, 1 Eq, 3.8 mmol) and triethylamine (1.9 g, 2.7 mL, 5 Eq, 19 mmol). After 5 min, a solution of 9-((tert-butyldiphenylsilyl)oxy)nonanoic acid (10, 1.9 g, 1.2 Eq, 4.6 mmol) in dichloromethane (1 ml) and PyBOP were added. Continued stirring for 5 h. The mixture was washed with 2 M NaOH (2×40 ml), then brine, dried over sodium sulfate and concentrated. The crude material of compound 11 was used as such.

The material was dissolved in THF (50 ml) and a 1M solution of tetrabutylammonium fluoride (5.7 mL, 1.5 Eq, 5.7 mmol) in THF was added and stirring continued for 16 h at room temperature. The mixture was concentrated and dissolved in ethyl acetate (250 mL) and washed with water (3×50 mL), then brine. The organics were dried over sodium sulfate and concentrated. Purified using flash (gradient 2% to 8% 7M NH₃ in MeOH in dichloromethane) to provide compound 12 as an oil, still containing PyBOP-residues. The material was dissolved in dichloromethane (50 mL) and added 2 M HCl in diethyl ether (4 mL) and stirred for 30 min. The white solids were filtered and washed with dichloromethane and ethyl acetate. The white solids were collected and partitioned between ethyl acetate (150 ml) and 1 M aqueous sodium hydroxide. The organic layer was dried over sodium sulfate and concentrated. Compound 12 (1.38 gram, 1.6 mmol) was isolated as a sticky oil.

2-Cyanoethyl (9-((3-(4-(3-(((8R,9S,13S,14S,17S)-17-(3-((1,1,1,3,3,3-hexafluoro-2-(trifluoromethyl)propan-2-yl)oxy)propoxy)-13-methyl-7,8,9,11,12,13,14,15,16,17-decahydro-6H-cyclopenta[a]phenanthren-3-yl)oxy)propyl)piperazin-1-yl)propyl)amino)-9-oxononyl) diisopropylphosphoramidite (1):

To a solution of compound 12 (1.38 g, 1 Eq, 1.55 mmol) and diisoropyl ammonium tetrazolide (293 mg, 1.1 Eq, 1.71 mmol) in dichloromethane (50 mL) was added at 0° C. 3-((bis(diisopropylamino)phosphaneyl)oxy)propanenitrile (937 mg, 2 Eq, 3.11 mmol). The mixture was stirred for 16 h at room temperature, then concentrated. Further purification using flash chromatography (very slow gradient 30% to 60% acetone in heptane (+1% Et₃N in all)) provided compound Apo-Si-K-1013 (830 mg, 0.76 mmol) and a lesser pure fraction (100 mg, 0.09 mmol) as a clear oil.

### Oligoribonucleotide Synthesis

Oligoribonucleotides were synthesized on solid phase according to the phosphoramidite technology employing a Mermade 12 synthesizer (LGC Bioautomation) at a synthesis scale of about 2×55 μmol per sequence (2 columns á 55 μmol). Syntheses were performed on a solid support made of controlled pore glass either loaded with N-Benzoyl deoxycytidine (CPG, 679 \acute{Å}, with a loading of 84 μmol/g) or N-Benzoyl-2′-O-Methyl-adenosine (CPG, 497 \acute{Å}, with a loading of 85 μmol/g). Regular DNA and RNA phosphoramidites, as well as ancillary reagents were purchased from SAFC Proligo (Hamburg, Germany). Specifically, the following amidites were used: (5′-O-dimethoxytrityl-N6-(benzoyl)-2′-O-t-butyldimethylsilyl-adenosine-3′-O-(2-cyanoethyl-N,N-diisopropylamino) phosphoramidite, 5′-O-dimethoxytrityl-N4-(acetyl)-2′-O-t-butyldimethylsilyl-cytidine-3′-O-(2-cyanoethyl-N,N-diisopropylamino) phosphoramidite, (5′-O-dimethoxytrityl-N2-(isobutyryl)-2′-O-t-butyldimethylsilyl-guanosine-3′-O-(2-cyanoethyl-N,N-diisopropylamino) phosphoramidite, 5′-O-dimethoxytrityl-2′-O-t-butyldimethylsilyl-uridine-3′-O-(2-cyanoethyl-N,N-diisopropylamino) phosphoramidite, (5′-O-dimethoxytrityl-N6-(benzoyl)-2′-O-methyl-adenosine-3′-O-(2-cyanoethyl-N,N-diisopropylamino) phosphoramidite, 5′-O-dimethoxytrityl-N4-(acetyl)-2′-O-methyl-cytidine-3′-O-(2-cyanoethyl-N,N-diisopropylamino) phosphoramidite, (5′-O-dimethoxytrityl-N2-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. In order to introduce 5′-monophosphates the Phosphat-On reagent available from ChemGenes (CLP-1544) was used. The Apo-Si-K170A building block was dissolved in 70% anhydrous DCM in anhydrous acetonitrile (100 mM) containing molecular sieves (3A). All other building blocks were dissolved in anhydrous acetonitrile (100 mM) containing molecular sieves (3 Å) except 2′-O-methyl-uridine phosphoramidite which was dissolved in 30% anhydrous DCM in anhydrous acetonitrile. Iodine (50 mM in pyridine: H₂O=9:1) was used as oxidizing reagent. 5-Ethyl thiotetrazole (ETT, 500 mM in acetonitrile) was used as activator solution. Coupling times were 5 minutes except when stated otherwise. Both, the Apo-Si building block, and the 5′-phosphate were incorporated into the sequence employing a double coupling step with a coupling time of 11 minutes per each coupling (total coupling time 22 min). The oxidizer contact time for these two building blocks has been extended to 2.5 min whereas the standard oxidizer contact time was set to 1.5 min.

Sequences were synthesized without removal of the final DMT group.

### Cleavage and Deprotection:

After assembly of the oligoribonucleotide sequences, the cyanoethyl protecting groups were cleaved. The CPG from both columns of each sequence were combined and treated with NH3:EtOH=3:1 (15 ml) for 18 hours at 45° C. The resin was filtered off and washed with 20% ethanol (2×5 ml).

TBDMS protecting groups were subsequently removed at elevated temperature using triethylamine hydrogen fluoride complex. The deprotection reaction was quenched by addition of H2O. The crude mixtures were then filtered off (ZapCap Nylon 0.2 μm bottle-top filters) and the filter was thoroughly washed with H₂O. The crude mixtures were adjusted to 100 mM triethylammonium acetate (TEAAc) and filled up to 300 ml (X69346K2) and 400 ml (X69347K2).

### Purification:

Crude oligomers were purified by RP HPLC using a 16×150 mm column (Dr. Maisch) packed with Source RPC resin (GE Healthcare) on an ÄKTA Pure instrument (GE Healthcare). Buffer A was 100 mM triethylammonium acetate (TEAAc, pH 7) and buffer B contained 95% acetonitrile in buffer A. A flow rate of 7.2 mL/min and a temperature of 60° C. were employed. UV traces at 260 and 280 nm were recorded. A gradient of 20% B to 100% B within 48 column volumes was employed. Appropriate fractions were pooled and precipitated in the freezer with 3M NaOAc, pH=5.2 and 85% Ethanol. Pellets were isolated by centrifugation, redissolved in water (50 ml), treated with 10×PBS buffer pH 7.4 (3 ml) and desalted by Size exclusion HPLC on an Akta Pure instrument using a 50×165 mm ECO column (YMC, Dinslaken, Germany) filled with Sephadex G25-Fine resin (GE Healthcare).

### Annealing:

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The requested aliquots were lyophilized for four days and stored at −20° C.

### Analytical Methods:

Crude single strands were analyzed by analytical LC-MS on a 2.1×50 mm XBridge column (Waters) on a Dionex Ultimate 3000 (Thermo Fisher Scientific) HPLC system combined with a LCQ Deca XP-plus Q-ESI-TOF mass spectrometer (Thermo Finnigan).

Buffer A was 16.3 mM triethylamine, 100 mM hexafluoroisopropanol (HFIP) in 1% MeOH in H2O and buffer B contained 95% MeOH in buffer A. A flow rate of 250 μl mL/min and a temperature of 60° C. were employed. UV traces at 260 and 280 nm were recorded. A gradient of 1-40% B within 0.5 min followed by 40 to 100% B within 13 min was employed.

Final single strands were analyzed by analytical LC-MS on a 2.1×50 mm XBridge column (Waters) on a Dionex Ultimate 3000 (Thermo Fisher Scientific) HPLC system combined with a Compact ESI-Qq-TOF mass spectrometer (Bruker Daltonics).

Buffer A was 16.3 mM triethylamine, 100 mM hexafluoroisopropanol (HFIP) in 1% MeOH in H2O and buffer B contained 95% MeOH in buffer A. A flow rate of 250 μl mL/min and a temperature of 60° C. were employed. UV traces at 260 and 280 nm were recorded. A gradient of 1-100% B within 31 min was employed.

Final duplexes were analyzed by analytical LC-MS on a 2.1×50 mm XBridge column (Waters) on a Dionex Ultimate 3000 (Thermo Fisher Scientific) HPLC system combined with a Compact ESI-Qq-TOF mass spectrometer (Bruker Daltonics).

Buffer A was 16.3 mM triethylamine, 100 mM hexafluoroisopropanol (HFIP) in 1% MeOH in H2O and buffer B contained 95% MeOH in buffer A. A flow rate of 250 pI mL/min and a temperature of 60° C. were employed. UV traces at 260 and 280 nm were recorded. A gradient of 1-100% B within 31 min was employed.

Prior to duplexes analysis, the disulfide bonds of the Apo-Si-K170A building block of both, the duplex, and each single strand were reduced in situ. To each of the analytical samples (50 μM in 100 mM TEAAc, 50 μl) were added 5 μl of a 100 mM solution of D,L-Dithiothreitol (DTT) in 100 mM triethylbicarbonate buffer (TEAB, pH 8.5) and allowed to stand at room temperature for at least 1 h prior to analysis.

### Example 2

### Cell-Free Silencing of EGFP Using Exemplary Conjugates of the Invention Methods

The dsiRNA Duplex: The siRNA Duplex was a Dicer substrate, designed to silence the EGFP gene.

The nucleotide sequences of the Apo-Si-K-170-A, Apo-Si-K-170-B, Apo-Si-K-170-C, Apo-Si-K-941, Apo-Si-K-1000, Apo-Si-K-1007, Apo-Si-K-1013 and Apo-Si-K-1014 Conjugates are as follows:

Sense:
(SEQ ID NO: 1)
5′-Phosphate(Apo-Si-K-170A)
ACCCTGAAGTTCATCTGCACCACCG-3′
Anti-sense:
(SEQ ID NO: 2)
5′-Phosphate(Apo-Si-K-170A)
CGGTGGTGCAGATGAACTTCAGGGTCA-3′
Sense:
(SEQ ID NO: 1)
5′-Phosphate(Apo-Si-K-170B)
ACCCTGAAGTTCATCTGCACCACCG-3′
Anti-sense:
(SEQ ID NO: 2)
5′-Phosphate(Apo-Si-K-170B)
CGGTGGTGCAGATGAACTTCAGGGTCA-3′
Sense:
(SEQ ID NO: 1)
5′-Phosphate(Apo-Si-K-170C)
ACCCTGAAGTTCATCTGCACCACCG-3′
Anti-sense:
(SEQ ID NO: 2)
5′-Phosphate(Apo-Si-K-170C)
CGGTGGTGCAGATGAACTTCAGGGTCA-3′
Sense:
(SEQ ID NO: 1)
5′-Phosphate(Apo-Si-K-941)
ACCCTGAAGTTCATCTGCACCACCG-3′
Anti-sense:
(SEQ ID NO: 2)
5′-Phosphate(Apo-Si-K-941)
CGGTGGTGCAGATGAACTTCAGGGTCA-3′
Sense:
(SEQ ID NO: 1)
5′-Phosphate(Apo-Si-K-1000)
ACCCTGAAGTTCATCTGCACCACCG-3′
Anti-sense:
(SEQ ID NO: 2)
5′-Phosphate(Apo-Si-K-1000)
CGGTGGTGCAGATGAACTTCAGGGTCA-3′
Sense:
(SEQ ID NO: 1)
5′-Phosphate(Apo-Si-K-1007)
ACCCTGAAGTTCATCTGCACCACCG-3′
Anti-sense:
(SEQ ID NO: 2)
5′-Phosphate(Apo-Si-K-1007)
CGGTGGTGCAGATGAACTTCAGGGTCA-3′
Sense:
(SEQ ID NO: 1)
5′-(Apo-Si-K-1013)
ACCCTGAAGTTCATCTGCACCACCG-3′
Anti-sense:
(SEQ ID NO: 2)
5′-(Apo-Si-K-1013)
CGGTGGTGCAGATGAACTTCAGGGTCA-3′
Sense:
(SEQ ID NO: 1)
5′-(Apo-Si-K-1014)
ACCCTGAAGTTCATCTGCACCACCG-3′
Anti-sense:
(SEQ ID NO: 2)
5′-(Apo-Si-K-1014)
CGGTGGTGCAGATGAACTTCAGGGTCA-3′

Protein-Free Fraction Upon Incubation with BSA

In order to perform systemic administration into the blood, it is valuable for a drug to have both a fraction that is bound to the serum/plasma proteins, and a free-fraction, free to migrate through the extracellular space into the cells. In order to examine this aspect for the Apo-Si-K-170-A, Apo-Si-K-170-B, and Apo-Si-K-170-C Conjugates, gel electrophoresis was employed: 20 pmole of Apo-siRNA Constructs samples were diluted in Tris buffer pH=7.4, and bovine serum albumin (BSA) was added to a final concentration of 2 mg/ml and 5 mg/ml. All samples were incubated over night at 37° C. These Samples were then loaded on 12% native polyacrylamide gel and migrated on electrical field for 1 hour in 5V/cm (bio-rad mini protean). Control samples included dsi-RNA Constructs samples diluted in water rather than in BSA. Apo-Si—Si, a structurally similar Apo-Si Conjugate that binds with high affinity to BSA was used as positive control. The chemical structure of Apo-Si—Si is presented below:

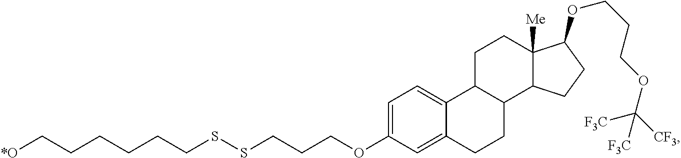

where * presents an attachment point to oligonucleotide.

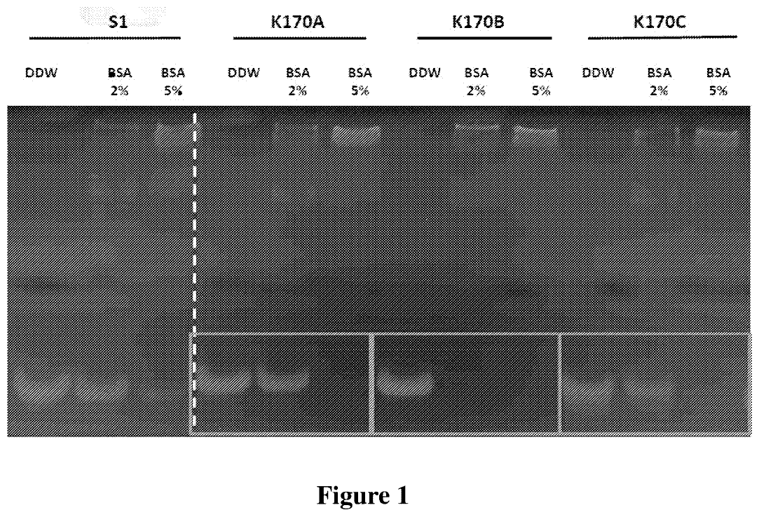

Results: FIG. 1 demonstrates protein-free fraction upon incubation with BSA. In contrast to Apo-Si—Si, that interacted almost completely with the BSA (>95% bound), substantial free fractions were observed of the tested conjugates in which Apo-Si-K-170-A shows a higher free fraction in comparison to Apo-Si-K-170-B and Apo-Si-K-170-C.

Moreover, the inventors have observed that Apo-K-160-A,

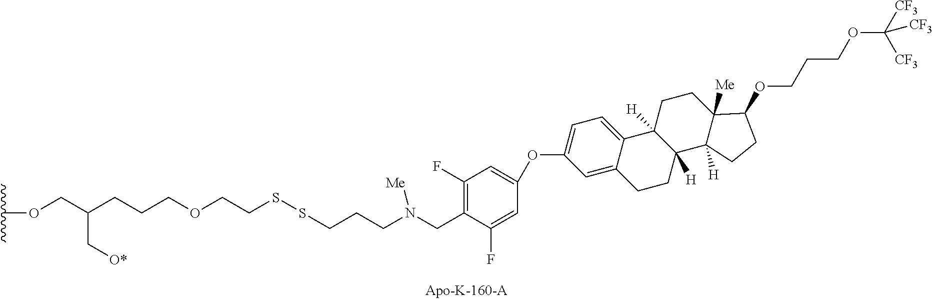

which is a close structural analog of Apo-Si-K-170-A, was inactive in the presence of serum (no GFP inhibition in HeLa-GFP cells was observed upon incubation with Apo-K-160-A even at a concentration of 600 nM in the presence of serum).

Thus, the inventors have concluded that the conjugates of the invention have significantly lower binding affinity to serum proteins (e.g. BSA) compared to structurally similar analogs not according to the invention.

Accordingly, it is presumed that that the conjugates of the invention will have greater potency in-vivo (compared to structurally similar conjugates) due to the lower unspecific binding to serum proteins, especially when administered iv.

Cleavage of the Disulfide Moiety of the Apo Conjugates Upon Incubation with—glutathione (GSH)

One of the hallmarks in the design of the Apo-Si-K-170-A, Apo-Si-K-170-B and Apo-Si-K-170-C moieties was incorporation of a disulfide bond, aimed to undergo cleavage selectively in the reductive conditions that prevail within the cytoplasm, thus releasing the cargo genetic drug to interact with the cytoplasmatic gene silencing complexes: Dicer and RISC. In order to demonstrate this feature, 20 pmole of RNA samples were diluted in 30 mM Tris buffer pH=7.4, supplemented with 5 mM of Glutathione (GSH, Sigma). All samples were incubated for 4 hours at 37° C. Control samples were diluted in water. Samples were then loaded on 12% native polyacrylamide gel and migrated on electrical field for I hour in 5V/cm (Bio-Rad mini protean).

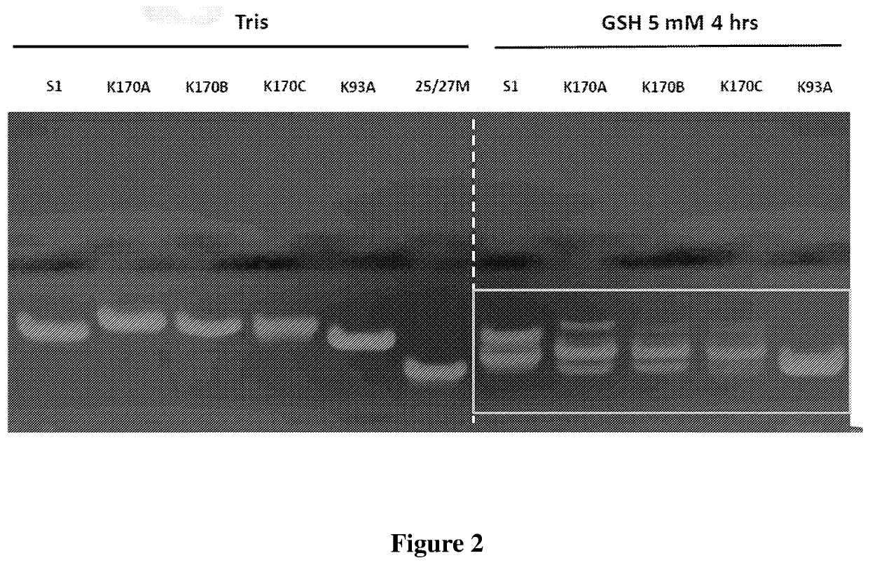

Results: FIG. 2 demonstrates that incubation of the Apo-Si-K-170-A, Apo-Si-K-170-B and Apo-Si-K-170-C conjugates with glutathione (5 mM for 4 hours in 37° C.)led to a robust cleavage of the conjugates in the following order: Apo-Si-K-170-C>Apo-Si-K-170-A>Apo-Si-K-170-B<<Apo-Si-K-93-A.

Apo-Si-K-93-A (the chemical structure is presented below) is a structurally similar conjugate having a disulfide bond. Accordingly, based on the results presented in FIG. 2, the inventors have concluded that the conjugates of the invention are characterized by a superior ability to undergo cytoplasmic disulfide cleavage, compared to structurally similar conjugates.

### Apo-Si-K-93-A:

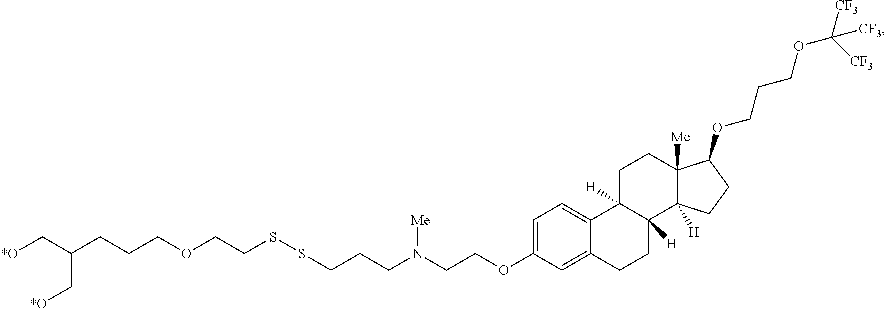

wherein * is an attachment point to the oligonucleotide, H, or phosphate residue.

### Example 3

In-Vitro Silencing of EGFP Using Exemplary Conjugates of the Invention Cell culture

Studies in vitro: Hela-GFP and 3T3-GFP cell lines were obtained from Cell Biolabs. Cells were grown in Dulbecco's modified Eagle's medium (Gibco) supplemented with 10% FBS (Gibco), 100 U/ml penicillin 100 mg/ml streptomycin (Biological Industries, Israel) and blasticidin 10 μg/ml. Cells were maintained in a 37° C. incubator, with 5% CO₂ humidified air. One day before transfection, cells were plated (40,000 cells/well) on 24-well black glass bottom plates. The following day, cells were exposed to Apo-Si-K-170-A, Apo-Si-K-170-B, and Apo-Si-K-170-C conjugated to EGFP-dsiRNA (see above) in the presence of complete medium containing 10% serum. For serum-free transfections conditions, medium was aspirated, cells were washed with Hank's Balanced Salt Solution (HBSS), and medium was then replaced with serum free Opti-MEM (Thermo Fisher Scientific) for 24 hours, followed by addition of complete medium for incubation for additional 48 hours. Down-regulation of protein expression was measured at 72 hours post transfection. For this purpose, medium was aspirated, and cells were washed with HBSS. EGFP fluorescence intensity was quantified by the Infinite M200-Pro Multimode Reader (Tecan), excitation wavelength 488 nm, emission wavelength 535 nm. Untreated cells were used as Controls. Experiments were performed in triplicates; results are presented as mean+SD.

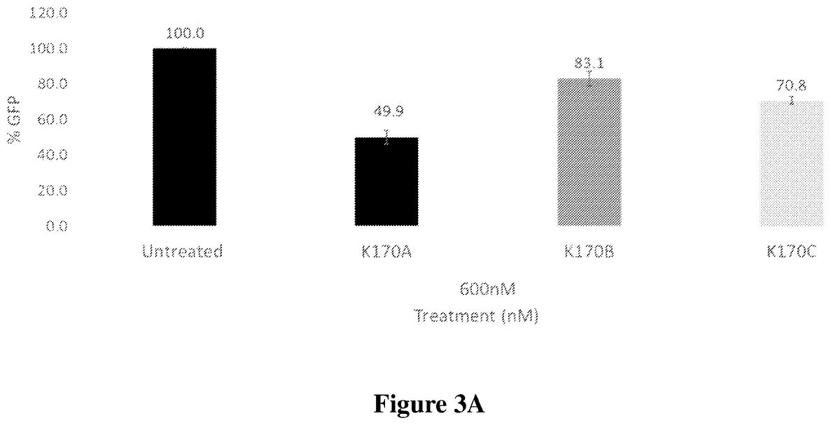

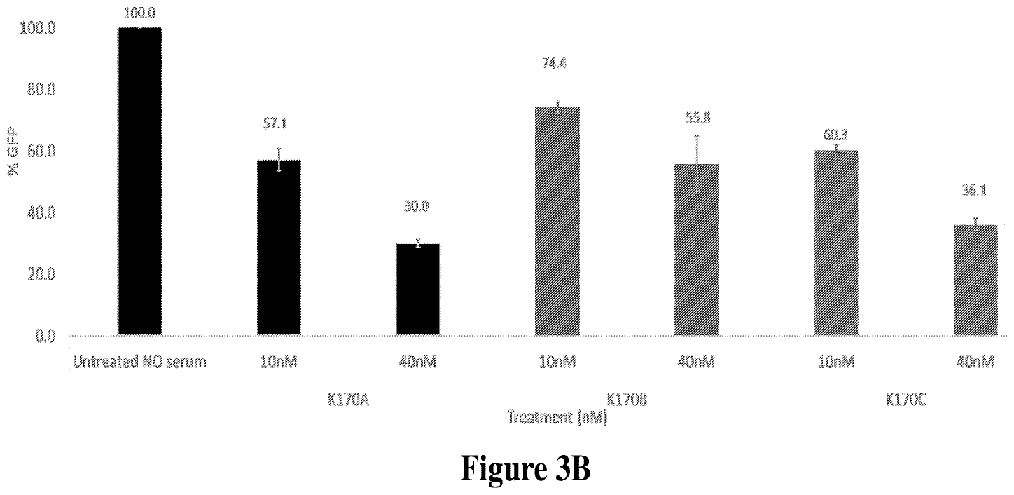

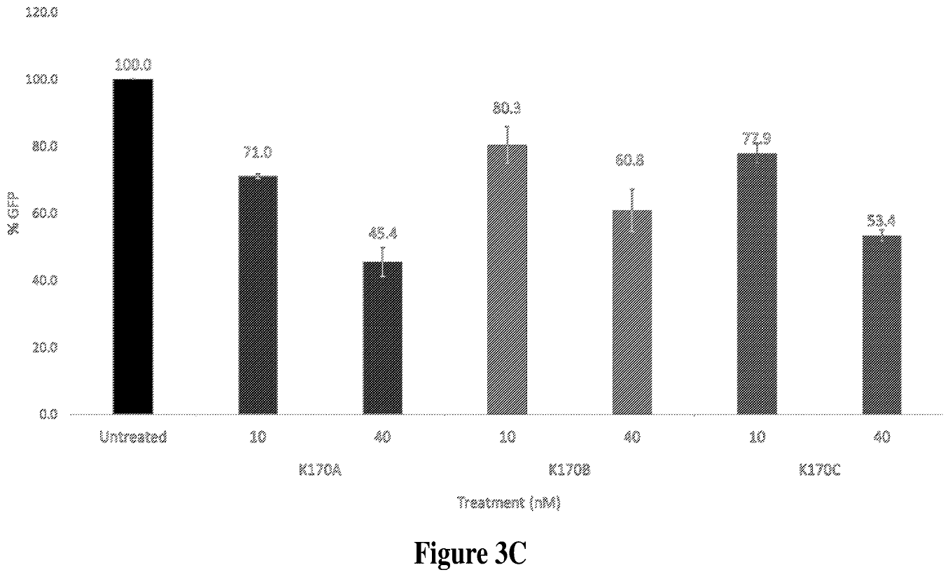

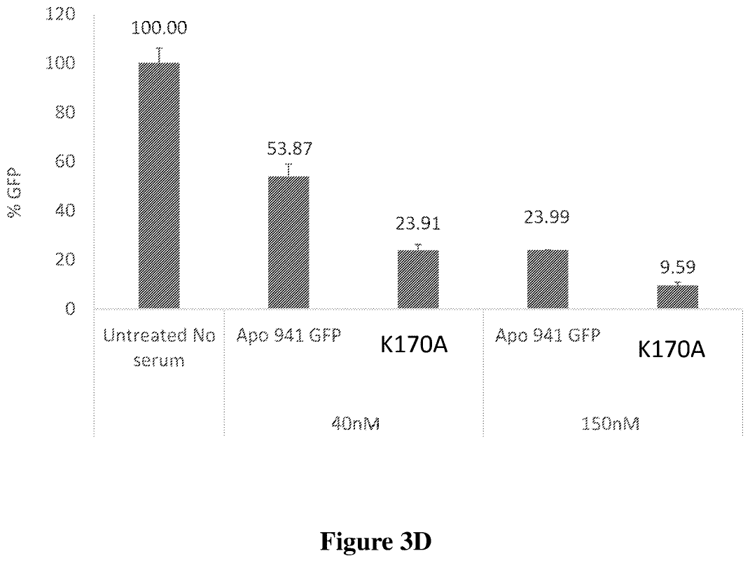

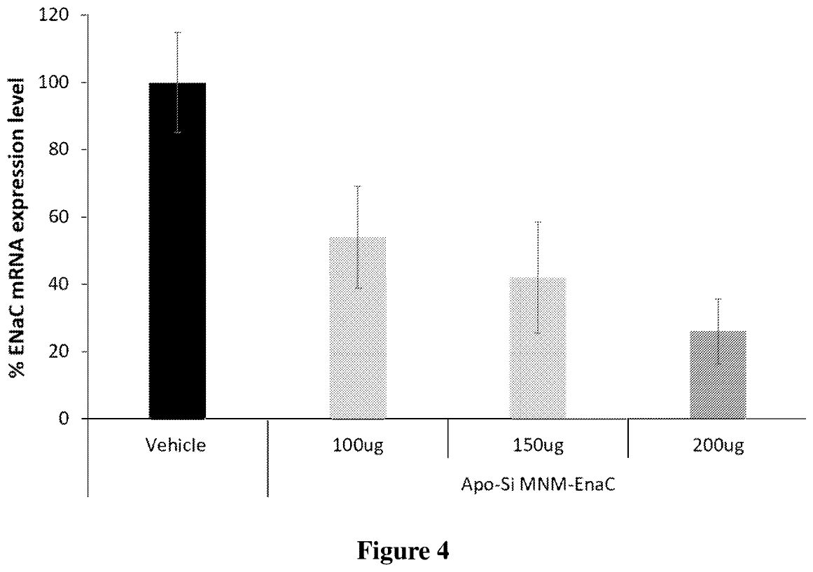

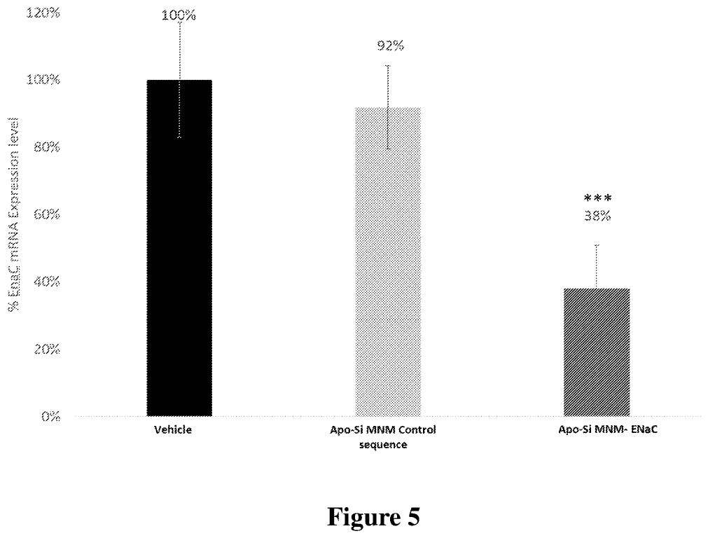

Results: In the presence of complete medium (10%)serum in Hela-GFP cell line, 600 nM of the Conjugate Apo-Si-K-170-A, Apo-Si-K-170-B and Apo-Si-K-170-C reduced EGFP expression to 49.9%, 83.1% and 70.8% of Control, respectively (see FIG. 3A).

In serum-free conditions, a robust EGFP knockdown was induced by the Apo-Si-K-170-A Conjugate, in a dose-dependent manner, with the EGFP expression being reduced to 57.1% and 30.0% of Control, when cells were treated by 10 and 40 nM of the Conjugate, respectively. Same trends can be observed for the Apo-Si-K-170-B and Apo-Si-K-170-C Conjugates (see FIG. 3B).

In a 3T3-GFP cell line under serum-free conditions, a significant EGFP knockdown was induced by the Apo-Si-K-170-A Conjugate, in a dose-dependent manner, with the EGFP expression being reduced to 71.0% and 45.5% of untreated control, when cells were treated by 10 and 40 nM of the Conjugate, respectively. Same trends can be observed for the Apo-Si-K-170-B and Apo-Si-K-170-C Conjugates (see FIG. 3C).

Conclusions: The Apo-Si-K-170-A Conjugate has improved silencing capability over the Apo-Si-K-170-B and Apo-Si-K-170-C Conjugates in the Hela-GFP cell line in the presence of serum.

Results: In 3T3-GFP cell line under serum-free conditions, a robust EGFP knockdown was induced by the Apo-Si-K-941 Conjugate, in a dose-dependent manner, with the EGFP expression being reduced to 53.87% and 23.99% of untreated control, when cells were treated by 40 and 150 nM of the Conjugate, respectively. Same trends were observed for the Apo-Si-K-170A Conjugates (see FIG. 3D).

Conclusion: The Apo-Si-K-170-A Conjugate has an increased silencing capability over the Apo-Si-K-491 Conjugates in the 3T3-GFP cell line in serum-free conditions.

Furthermore, Apo-Si-K-1013 based conjugate showed robust downregulation of EGFP in-vivo (data not shown).

### Example 4

### In-Vivo CF Efficacy

Cystic Fibrosis (CF) is a genetic disease that causes limits the ability to breathe and coughing up mucus as a result of frequent lung infections (>70,000 worldwide, 1,000 new cases each year). Mutations in the CF Transmembrane Conductance Regulator (CFTR) gene cause the CFTR protein to become dysfunctional and ions cannot flow out of the cell due to a blocked channel. Without the chloride (Cl⁻) to attract water to the cell surface, the mucus in various organs becomes thick and sticky. Epithelial sodium channel (ENaC) is a membrane-bound ion channel that is selectively permeable to Na⁺. ENaC is composed of three homologous subunits (a,b,g) encoded by four genes: SCNN1A/B/G/D. In the absence of functional CFTR, the ENaC channel is upregulated, and further decreases salt and water secretion by reabsorbing sodium ions. As such, the respiratory complications in CF are not solely caused by the lack of chloride secretion but instead by the increase in sodium and water reabsorption; aENaC subunit is required for full channel function and b as well as g subunits are regulators of ENaC activity and residual ENaC activity can be measured in their absence. Exemplary inhaled conjugate of the invention (K170A-ENaC conjugate) is designed to block the absorption of sodium that may keep the surface of the airways hydrated, which may help make mucus less thick, making it easier to keep airways clear in CF patients. Moreover, ENaC inhibition may act synergistically with CFTR modulators.

The sequences of K170A-ENaC conjugate areas follows:

Sense:
(SEQ ID NO: 3)
5′-Phosphate-(Apo-Si-K170A)TGTGCAACCAGAACAAATCAGAC
TG-3′
Antisense:
(SEQ ID NO: 4)
5′-Phosphate-(Apo-Si-K170A)CAGTCTGATTTGTTCTGGTTGCA
CAGT-3′.

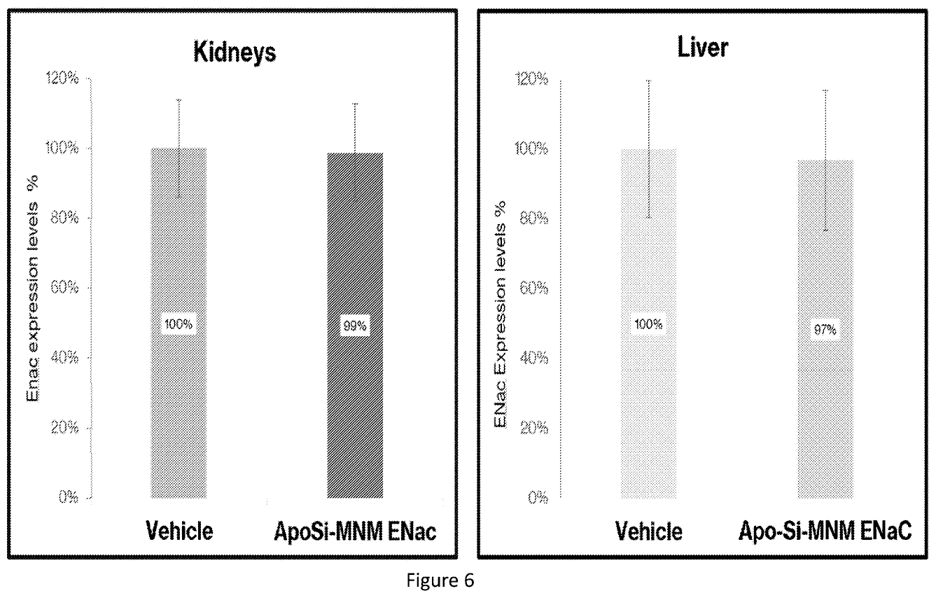

CF efficacy evaluation in mouse model: Using K170A-ENaC conjugate to dicer substrate siRNA (K170A-ENaC conjugate, 400 nM) leads to 63% knockdown (KD) of the SCN1A gene in adenocarcinomic human alveolar basal epithelial cells (A549) after dexamethasone induction as well in Hela transfected cells (75%). Next, native female ICR mice were treated IT using microspore with substance (100-200 ug) in 50 μL of 5% glucose on day 1,2,5 and terminated on day 7 Knock-down evaluation by real time PCR analysis. To note, only 50% KD of ENaC is required in CF patients. The in-vivo experiment shows high KD using K170A-ENaC conjugate in a dose-dependent manner (100 μg—46%, 150 μg—58%, 200 μg—76%), while 200 μg of siRNA alone or 200 μg of K170A-ENaC conjugate to dicer substrate siRNA-GFP as a control non-related sequence, leads to 15% KD only. These results indicate high selectivity and a high potential of K170A-ENaC conjugate as a treatment for CF. As can be seen in FIG. 6, upon local delivery there is no systemic exposure as presented in the liver and kidney of MNM-siRNA and ENaC KD is specific to the target tissue. Upon entry into the cell, the reducing environment of the cytoplasm causes detachment of the MNMs, and the siRNA is trapped in the cell. The MNMs are metabolized and excreted, while the RNA is trimmed by Dicer, single RNA strands enter the RNA-induced silencing complex (RISC) to effect cleavage of complementary mRNAs.

### Example 5

### In-Vivo HL Efficacy

Hearing loss (HL) is an underestimated condition that affects more than 1.5 billion people globally. There are different types of HL but the most prevalent (90%) is sensorineural HL (SNHL). SNHL typically occurs following damage to or dysfunction of the hair cells within the inner ear; their synapses with primary auditory neurons (synaptopathy) or the vestibulocochlear nerve; of the stria vascularis or the brain's central processing centers. The cochlear distribution of Cy3-labeled dsiRNA conjugate of the invention was assessed in comparison to naked Cy3-labeled dsiRNA after intracochlear (IC) delivery in guinea pigs (GP).

The in vivo study consisted of five groups of GP: one (1) sham group (no treatment), one (1) group treated with Cy3-Naked dsiRNA by IC administration route (one ear), two (2) groups treated with Apo-Si-K170A Cy3-dsiRNA conjugate by IC administration route and sampled at different time points):
Group 1: Sham group (n=3)—both ears were tested
Group 2: Cy3-Naked dsiRNA treated group (IC route)—T_{+30HOURS} (14.4 μg/ear) (n=6)
Group 3: Apo-Si-K170A Cy3-dsiRNA conjugate treated group (IC route)—T_{+25HOURS} (14.4 μg/ear) (n=6)
Group 4: Apo-Si-K170A Cy3-dsiRNA treated group (IC route)—T_{+30HOURS} (14.4 μg/ear) (n=6)

The sequences of the Apo-Si-K170A Cy3-dsiRNA conjugate are as follows:

Sense:
(SEQ ID NO: 5)
5′-Phosphate(Apo-si-K170A)(Cy3)TTACCCTGAAGTTCATCTG
CACCACCG-3′.
Antisense:
(SEQ ID NO: 2)
5′-Phosphate(Apo-si-K170A)CGGTGGTGCAGATGAACTTCAGGG
TCA-3′.

The dsiRNA was delivered by IC infusion for 24 hours using an Alzet osmotic pump connected to a catheter inserted at the base of the cochlea. Animals were sacrificed 1 or 6 hours, post IC infusion (T_{+25HOURS} or T_{+3HOURS}, corresponding to 1 or 6 hours after 24 hours of continuous infusion), The distribution of Cy3-labeled MNMs-dsiRNA conjugate was qualitatively assessed (present or not present) in cochlear tissue at two time points after the end of delivery (1 or 6 hours), using two different histological techniques: flat surface preparation and cochlear cross sections. For the flat surface preparation, the distribution of the Cy3-labeled MNMs-dsiRNA conjugate and Cy3-naked dsiRNA was described in hair cells, in the supporting cells and in the auditory fibers for three fragments along the cochlear partition (apex, mid and base of the cochlea). For the cochlear cross sections, the distribution of the Cy3-labeled MNMs-dsiRNA and Cy3-dsiRNA was described in hair cells, in the supporting cells, in the auditory fibers and in the spiral ganglion neurons for three sections of the cochlea (apex, mid and base of the cochlea).

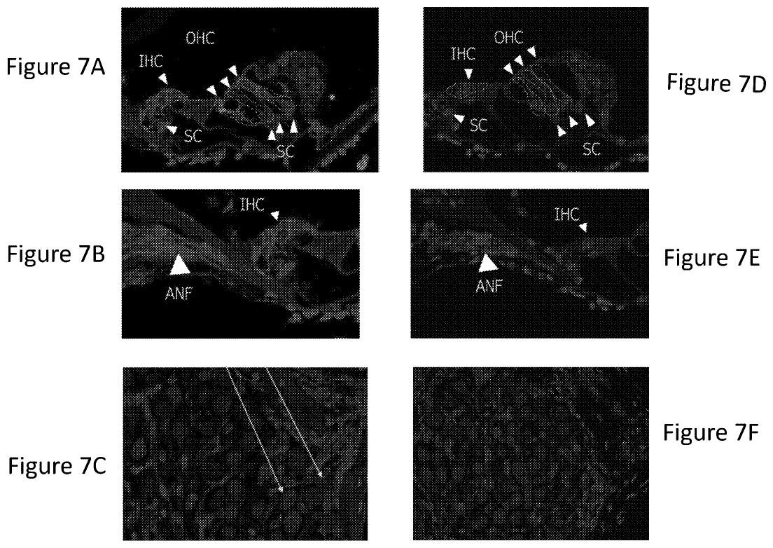

As can be seen in FIGS. 7A-C, an enhancement of the Cy3 signal was observed in the inner hair cells (IHC), in the outer hair cells (OHC), in the supporting cells (SC), in the auditory nerve fibers (ANF) and in the spiral ganglion cells (indicated by arrows) at the base, median and apex of the cochleae in the Apo-Si-K170A dsiRNA conjugate treated group (IC) T_{+30HOURS} (Group 4) compared to the Naked dsiRNA treated group (IC) T_{+30HOURS} (Group 2) as presented in FIGS. 7D-F.

### Example 6

### In-Vitro CMT1A Efficacy

### Materials and Methods

In vitro studies were performed with 3T3-NIH cells in 6-well plates (200,000 cell/well, 2 ml well). Test material (Apo-Si-K1000-PMP22 or Apo-Si-K1000-non-specific dsiRNA sequence) was diluted (10-400 nM) in OptiMEM medium from a 20 μM stock solution and incubated for 48 hr at 37° C./5% CO₂ in a humidified incubator. In serum-free conditions, prior to transfection with Apo-Si-MNM Construct, medium was removed, and the cell monolayer was washed once with a large volume of HEPES-buffer saline and replaced with OptiMEM.

The sequences of Apo-Si-K1000-PMP22 are as follows:

Sense:
(SEQ ID NO: 6)
5′-p(Apo-Si-K1000)GAAATGGTGCTATAGATTTACCATT-3.
Antisense:
(SEQ ID NO: 7)
5′-p(Apo-Si-K1000)AATGGTAAATCTATAGCACCATTTCAC-3′.

24 h post transfection, a fresh complete DMEM medium (10% FBS) with penicillin/streptomycin antibiotics was added. RNA was extracted using pure link RNA extraction mini kit, lysed with 1 ml of TRIzol reagent according to manufacturer protocol and its concentration measured by nanodrop for cDNA preparation. RT-PCR was performed by Fast SYBR green master mix protocol using specific probes against the target gene and beta-Actin as endogenous control.

A similar assay was performed in HeLa cells (40,000 cell/well, 2 ml well) with test material at a concentration range of 100-400 nM as well as in S16-Schwann cells (350,000 cell/well, 2 ml well) in test material concentration range of 50-200 nM.

Charcot Marie-Tooth type 1A (CMT1A) disease is believed to be caused by duplication of a region of chromosome 17 that encodes for overexpression of the Peripheral Myelin Protein 22 (PMP22 protein). PMP22 is an integral membrane protein, hydrophobic glycoprotein, with high expression mainly in Schwann cells. It is a major component of compact myelin in the peripheral nervous system and accounts for 2-5% of total protein content. It has been reported to play a key role in the maintenance of cholesterol homeostasis in Schwann cells. Therefore, a dsiRNA targeting PMP22 gene delivered using MNMs directly into the cytoplasm of Schwann cells, could prove a breakthrough in the efforts to develop a treatment for this disorder.

The inventors developed a PMIP22 targeting dsiRNA (MNM-PMP22) which is designed to be administered systemically or intrathecally.

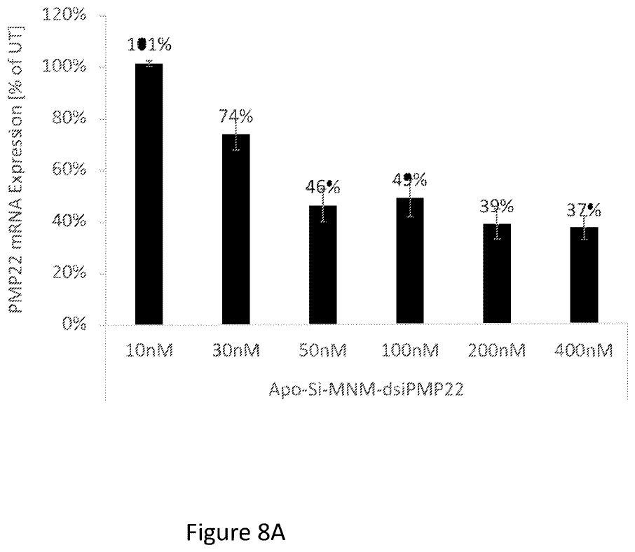

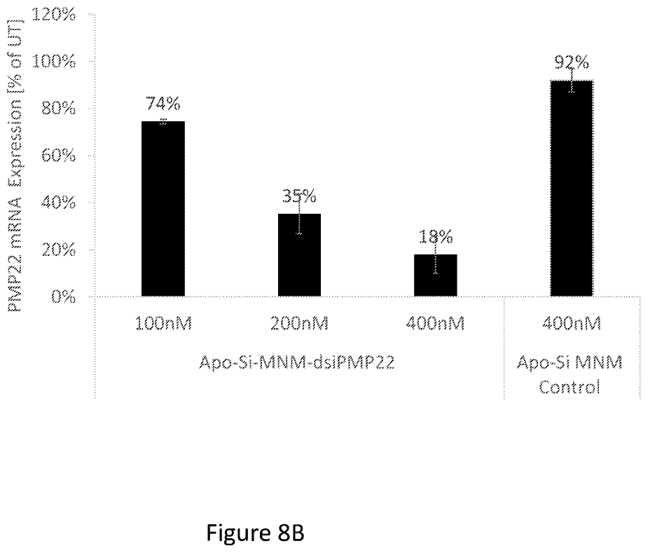

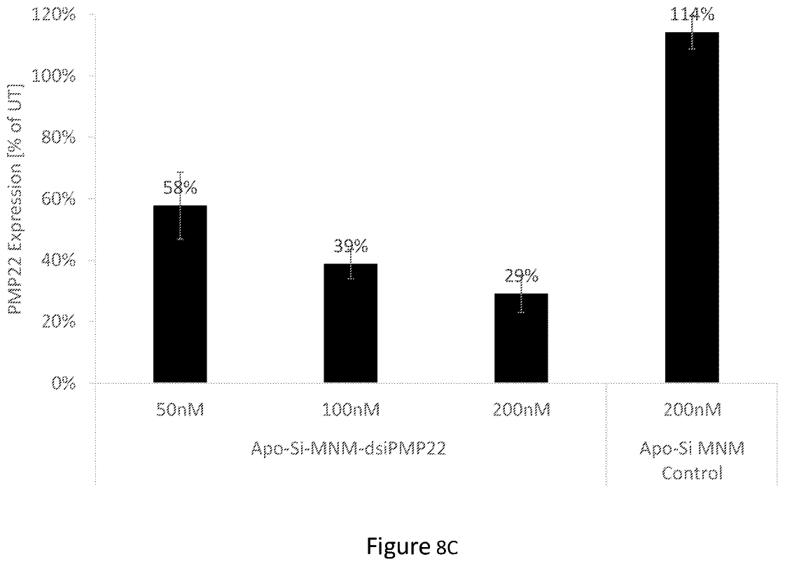

Initial in vitro studies performed by the inventors have demonstrated that Apo-Si-K1000-PMP22 is successful in silencing the expression of the PMP22 gene in a concentration-dependent manner in 3T3-NIH cells (see FIG. 8A). The IC₅₀ for this effect was ˜50 nM. Similar results were obtained in HeLa cells (see FIG. 8B) and in S16-Schwann cells (see FIG. 8C). In these studies, it was further demonstrated that the Apo-Si-K1000-PMP22 dsiRNA conjugate did not alter the expression of unrelated genes, demonstrating the specificity of this approach. Taken together, these results underscore the great potential of the Apo-Si dsiRNA delivery approach in the treatment of CMT1A.

### Example 7

### Anti-Viral Activity In Vivo

RSV: Apo-Si-K170A-V20, in the RSV mouse infection model. The female BALB/c mice infected with RSV were treated with vehicle, positive control compound (Ribavirin, 50 mpk) or test article with the predetermined regimens and dosages. Lung tissues were harvested on day 5 to determine the viral titers to evaluate the efficacy of RSV inhibition.

The chemical structure of Apo-Si-K170A-V20 is as follows:

Sense:
(SEQ ID NO: 8)
5′-p(Apo-Si-K170A) GGCTCTTAGCAAAGTCAAGTTGAAT-3′
Antisense:
(SEQ ID NO: 9)
5′-p(Apo-Si-K170A) ATTCAACTTGACTTTGCTAAGAGCCAT-3′

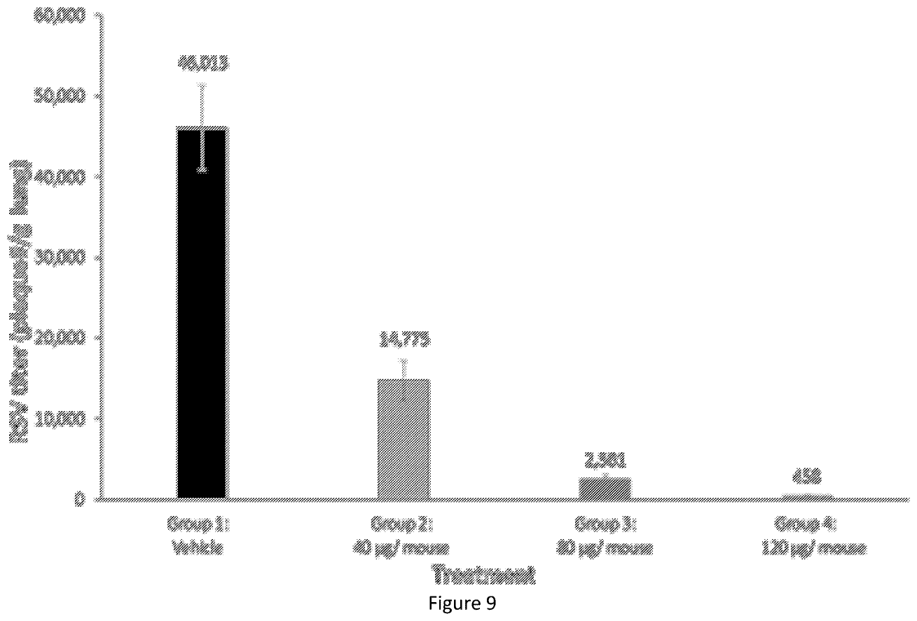

The results of the experiment are summarized in FIG. 9.

The positive control, ribavirin (50 mg/kg), significantly reduced the lung viral titer by 0.869 Log (plaques/g lung tissue, similarly hereinafter) when compared with vehicle group, which was in expectation and showed good consistency with the historical data. The test article Apo-Si-K170A-V20, reduced the lung viral titer by 0.563, 1.324, 1.966, and 1.549 Log, respectively, when dosed at 40, 80, and 120 μg/dose (as presented in FIG. 9), which suggested optimal efficacy against RSV and obvious dose-response under the set conditions. Conclusion, Apo-Si-K170A is highly effective against RSV infection in mouse model.

SARS-CoV-2: Efficacy testing of Apo-Si-MNMs-dsiRNA (also used herein as “drug”) administered by intranasal and intratracheal instillations against infection of SARS-CoV-2 virus in African Green Monkeys (AGM) were performed in two studies. In the first study, AGMs (n=10) were divided into two groups: Apo-Si-MNMs-dsiRNA (n=5) vs. vehicle-control (n=5). Animals were infected through Intranasal and Intratracheal delivery of the virus at Day 0 (total load 3×10e5) with SARS-Related CoV-2, Isolate USA-WA1/2020 (BEI Resources). Drug was administered as a prophylaxis treatment, in three daily consecutive doses at days −3, −2 and −1 pre infection. Doses of drug were 10 mg total; 2.5 mg delivered into each nostril and 5 mg delivered intratracheally. Animals were followed daily for clinical observations, body weights and body temperature.

The chemical structure of Apo-Si-MNMs-dsiRNA is as follows:

Sense:
(SEQ ID NO: 10)
5′ p-(Apo-Si-K170A) CTAAAGGACCTCACGAATTTTGCTC 3′
Antisense:
(SEQ ID NO: 11)
5′ p-(Apo-si-K170A) GAGCAAAATTCGTGAGGTCCTTTAGTA 3′

No clinical signs were observed during drug administration. No Test Article related changes in body weights and body temperatures were observed. At scheduled necropsy on Day 7, animals were grossly unremarkable in gross pathology. Terminal body weight-to-lung weight ratios was calculated and indicated no differences between groups. Viral load was evaluated by qRT-PCR.

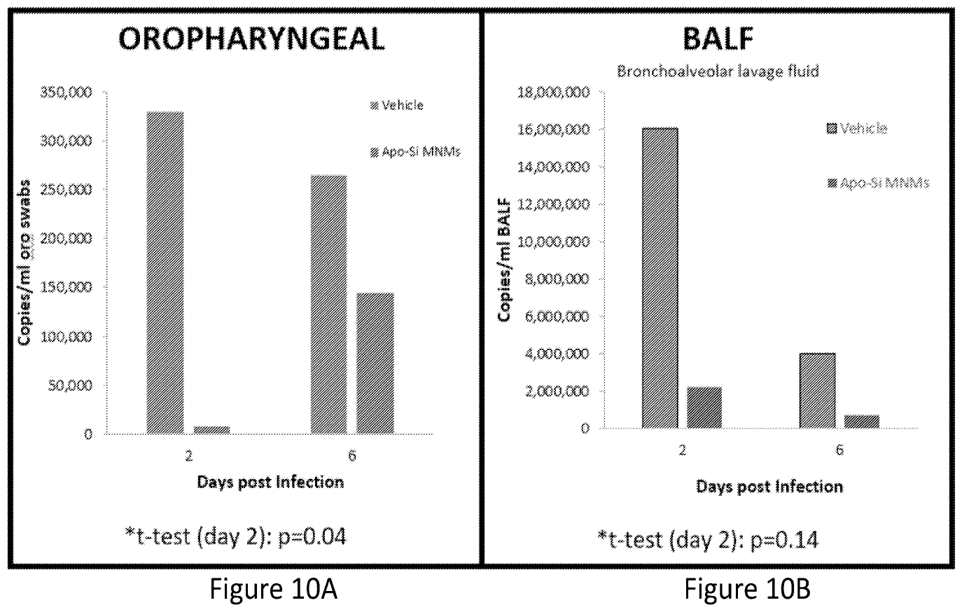

The results of the experiment are summarized in FIGS. 10A-B.

As presented in FIG. 10A, viral load, in oropharyngeal tissue at day 2 the results are statistically significant (p=0.04) in reduction of ˜2 logs in viral load, reflecting 98% gene knockdown. In bronchoalveolar lavage (BALF) reduction of ˜1 log in viral load observed at day 2, reflecting gene knockdown of 86% (FIG. 10B).

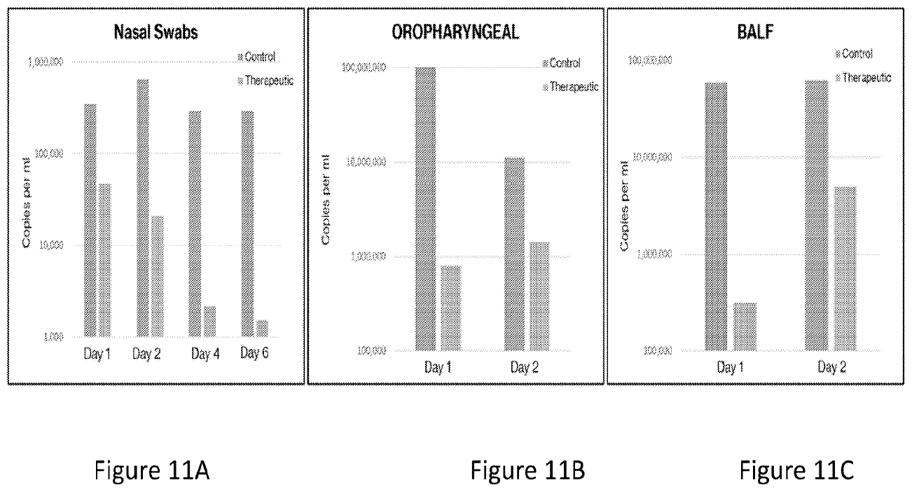

The second efficacy evaluation of SARS-CoV-2 in AGMs included therapeutic treatment using intranasal device and nebulization. Animals were infected at Day 0 with SARS-Related CoV-2, Isolate USA-WA1/2020 (BEI Resources). The therapeutic group received treatment at Day 0, 1, 2, 4. Animals were followed daily with clinical observations, body weights and body temperature. Similarly, to the first AGM efficacy study, no clinical signs were observed. As presented in FIGS. 11A-C qRT-PCR results demonstrated a reduction of ˜2 log in viral load in oropharyngeal tissue, ˜2 log reduction in the BALF and in the nostrils, there was a potent and long-lasting inhibition effect on viral load which resulted in ˜3 log reduction in viral load.

Conclusion: Apo-Si-K170A is highly effective against SARS-CoV-2 infection in AGM model.

### Example 8

### Asthma Evaluation in Mice Model

The inventors tested in vivo efficacy of an exemplary conjugate of the invention (Apo-Si-K170A-MNM) in OVA-induced asthma model in mouse. Asthma is a chronic airway inflammatory disease characterized by Airway hyperresponsiveness (AHR), airway inflammation and remodeling. OVA induced allergic asthma is a classic model and is often used to establish anti-asthma effects of test articles. The objective of the experiment disclosed hereinbelow was to evaluate the therapeutic efficacy of Apo-Si-K170A-MNM on targeting STAT6 gene in OVA-induced asthma model in mice.

Apo-Si-K170A STAT6 comprises of the following sequences:

Sense strand
(SEQ ID NO: 12)
5′-Phosphate(Apo-si-K170A) AGATGCTTTCTGTTACAACATGG
CC-3′;
and
Anti-sense strand
(SEQ ID NO: 13)
5′-Phosphate/(Apo-si-K170A) GGCCATGTTGTAACAGAAAGCT
CTGA-3′

### Ova Mice Model

Female Balb/C mice were randomized into 5 groups based on their body weight. Mice treatment groups were intraperitoneally (IP) injected with 100 μL OVA/Alum solution (0.30 mg/mL) on Day 0, 7 and 14, respectively. Sham group was injected 100 μL 1×PBS accordingly. On days 27-29, mice were challenged by 1% OVA solution (treatment groups) or PBS (sham control group) by atomization and administration for 30 min by DSI Buxco Mass.

Mice were treated with 200-250 μg of Apo-Si-K170A STAT6 Construct by intratracheal injection (IT) on day 26 and day 27 (6 hours prior to the first OVA challenge). Sham and vehicle control mice were administered with 5% glucose.

On day 29, mice were euthanized by exsanguination under deep anesthesia (a single dose of Zoletil 50 (50 mg/kg, 10 ml/kg) and Xylazine (5 mg/kg, 2 ml/kg) via IP injection).

### Cytokines Evaluation in BALF

On day 29, mice were euthanized, the trachea was surgically exposed, the right lung was lavage three times with 0.5 mL 1×PBS to gather the bronchoalveolar lavage fluid (BALF) together. The collected BALF sample was centrifuged at 1500 rpm for 10 min at 4° C. The supernatant was stored at −80° C. for subsequent cytokines test.

The levels of cytokines IL-4 and IL-13 in BALF supernatant were tested by Quantikine ELISA Kit (R&D systems M4000B, M1300CB).

### Total IgE Assay in Plasma

Blood was collected (on day 29) into EDTA-K2 tubes and immediately centrifuged at 3000 g for 10 min at 4° C. The plasma was collected and snap-frozen in liquid nitrogen. Total IgE level in plasma was quantitatively analyzed by ELISA KIT (Abcam GR3377142-1).

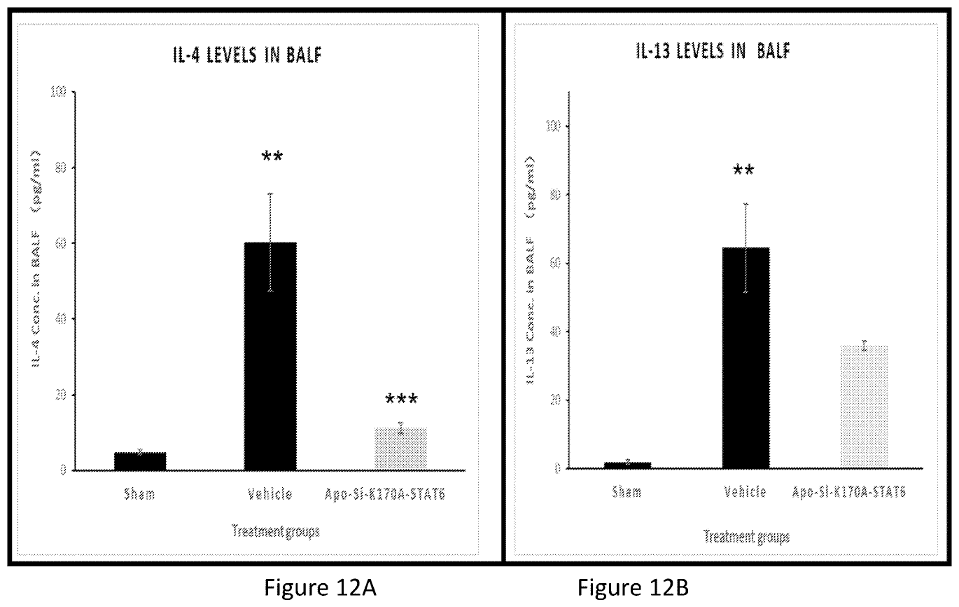

Surprisingly, the inventors observed that the exemplary conjugate of the invention induced down-regulation of th2 cytokines markers (Il-4 & Il-13) in BALF of asthma mice model. As presented in FIG. 12A, treatment with Apo-Si-K170A STAT6 significantly reduced IL-4 level, similar to sham. IL-13 levels was significantly increased (p<0.01) in vehicle group as compared to sham group, and reduced after treatment with Apo-Si-K170A STAT6 (Mean±SEM, N=1, n=6-12, **P<0.01, ***P<0.001 T-test) as presented in FIG. 12B.

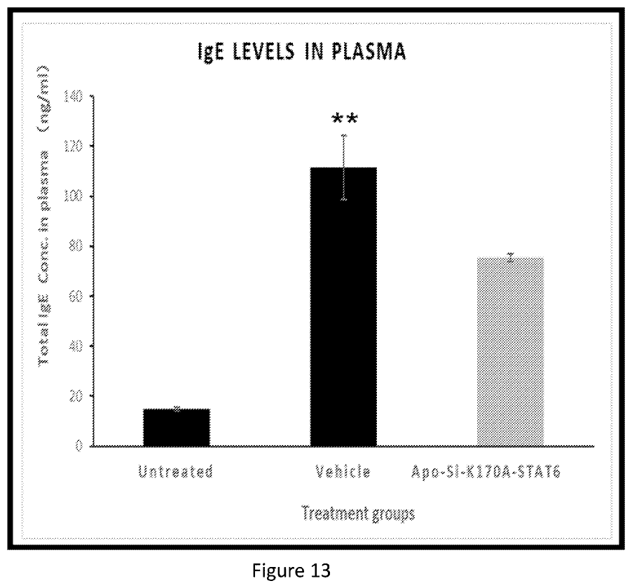

To this end, Apo-Si-K170A STAT6 construct inhibits IgE levels (see FIG. 13), and Th2 cytokines markers production in a significant difference compared with control mice.

### Example 9

### In-Vivo and In-Vitro Experiments in an Idiopathic Pulmonary Fibrosis Model

The inventors aimed to utilize an exemplary conjugate of the invention (Apo-Si-Apo-Si-K-170A-SPARC) for treatment of IPF and other obstructive pulmonary diseases (e.g. COPD). The inventors propose pulmonary administration of the conjugate for a direct delivery of the conjugate to the target site, thus preventing systemic exposure and possibly enhancing the efficacy of the drug.

Secreted Protein Acidic and Cysteine Rich (SPARC) is an important mediator of cell-matrix interaction and is indicated in playing a significant role in tissue fibrosis, by its high expression level in fibrotic diseases and stimulation of TGF-beta signaling. It is a secreted, acidic, extracellular matrix glycoprotein which plays a significant role in facilitating collagen fibril formation, which settle in the extracellular matrix and cause fibrosis. Studies performed in vitro and in vivo with siRNA-targeted knockdown of this gene show promising potential for its capacity to not only prevent collagen accumulation by lung cells, but also relieve chronic inflammation and fibrosis in bleomycin (BLM)-induced IPF mouse model.

Apo-Si-K-170A-SPARC is designed to downregulate the expression of SPARC in the lung, thus leading to a significant decrease in the generation of collagen fibrils and collagen accumulation in diseased lungs. The inventors postulate that Apo-Si-K-170A-SPARC may be effective in preventing progression of alveoli wall thickening, as well as in inducing a repair processes and clearance of fibrotic tissue.

Apo-Si-K-170A-SPARC is composed of dsiRNA conjugated on each of the strands to a selected Apo-Si-K-170A MNM moiety at the 5′-end, that following annealing form a duplex that has the following general structure:

Sense Strand
(SEQ ID NO: 14)
(5′-Phosphate/(Apo-Si-MNM-SPARC) CCACTTGAAACCTTCTA
CTAATCAA-3′
Anti-sense Strand
(SEQ ID NO: 15)
(5′-Phosphate/(Apo-Si-MNM-SPARC) TTGATTAGTAGAAGGTT
TCAAGTGGCA-3′.

### Evaluation of Various Apo-Si-K-170A Constructs in Vitro

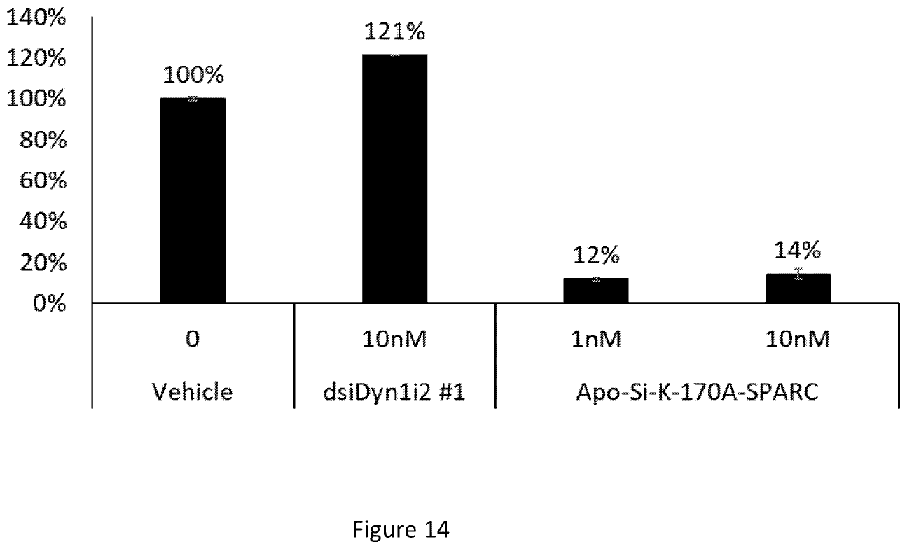

For selection of SPARC gene target sequences, several commercially available sequences were ordered (IDT trifecta products). Selected sequences were structurally modified and elongated according to M.A. Behlke's publications and IDT design manual in order to create a dicer substrate small interfering RNA (dsiRNA) 25/27 base pairs long sequence and modifications (Scott D. Rose and Mark A. Behlke. Chapter 2 Synthetic Dicer-Substrate siRNAs as Triggers of RNA Interference; 2012). Non-related negative control dsiRNA sequences were chosen from the literature or non-targeting related genes. Based on in vitro assessment of efficacy using Lipofectamine RNAiMax delivery system in several cell lines, dsiRNA sequence which showed the best performance in vitro (Apo-Si-K-170A-SPARC) was selected for further development (FIG. 14). Additional tested sequences also showed robust SPARC downregulation (not shown).

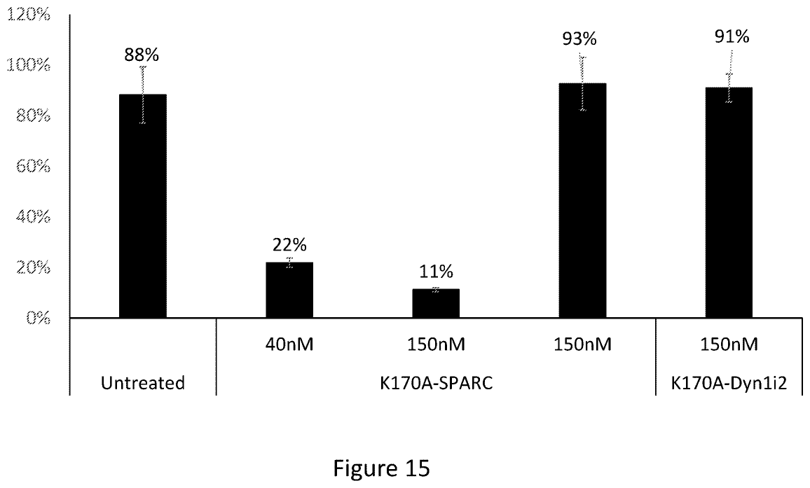

In order to evaluate the ability of the Apo-Si-MNM Constructs to downregulate the SPARC gene in cell lines, 3T3/NIH cells were transfected with the Apo-Si-MNM Constructs. Two days post transfection, the inhibitory effect of the Apo-Si-MNM was assessed by RNA extraction that was subjected to qPCR analysis. Lead candidate Constructs comprising the most potent dsiRNA sequence (Apo-Si-K-170A-SPARC) demonstrated significant and specific downregulation of the SPARC gene in a dose dependent manner (FIG. 15). By contrast, the negative control sequence (Apo-Si-K170A conjugate targeting a control gene, denoted as K170A-Dyn1i2) did not affect SPARC expression levels (FIG. 15).

### Evaluation of Apo-Si-K-170A-SPARC In Vivo

To evaluate the in vivo effect of Apo-Si-K-170A-SPARC, we established the Bleomycin-induced IPF model in C57BL mice. In this well characterized model, bleomycin (BLM) is administered by intranasal application (40 μL/mouse, 0.6 UI/Kg). BLM induces a strong inflammatory reaction in the lung that spans over a period of approximately 10 days, and is characterized by significant weight loss, elevated white blood cell count in lung exudate, and elevated levels of inflammatory cytokines, particularly related to TGF-β signaling. Towards the end of this period, the fibrotic stage of the disease occurs. Weight is often restored, but soluble collagen concentration in lung exudate increases and fibrotic tissue begins to form. Various inflammatory genes, as well as collagen-forming genes, are significantly upregulated in lung tissue mRNA.

To evaluate therapeutic effects of Apo-Si-K-170A-SPARC on the disease, we devised a therapeutic regiment initiated as of Day 4 after BLM administration. Apo-Si-K-170A-SPARC was administered by intra-tracheal (IT) injection compared to sham-treatment with vehicle, which served as a control.

In these studies, it was found that Apo-Si-K-170A-SPARC resulted in a significant knockdown effect of SPARC mRNA expression in diseased mice, which was even lower than SPARC expression in naïve mice, demonstrating the great potency of an exemplary conjugate of the invention.

### Example 10

### In-Vitro Experiments in Influenza a Virus Model

The inventors aimed to estimate the inhibitory activity of exemplary conjugates of the invention (Apo-Si-K170A-InfA constructs), targeting influenza A genes, PB1 and PB2, in vitro against influenza viruses on MDCK cell-based cytopathic effect (CPE) assays.

### Materials and Methods:

Using bioinformatical tools, we produced 30 siRNA sequences against the Influenza A PB1 and Influenza A PB2 genes (15 sequences per gene). siRNA were chosen based on the gene sequence of Influenza A/California/07/2009 (H1N1) strain and Influenza A/Perth/16/2009 (H3N2) virus strains. Each siRNA inhibitory effect was evaluated in a series of assays using 3T3/NIH transfected with a plasmid for the expression of PB1 or PB2, respectively to the evaluated siRNA and its source. Leading sequences for each gene were also cross-tested with a plasmid from the other virus strain. Finally, top sequences were modified according to Bhelke modifications and conjugated to Apo-Si-K170A molecular nanomotor, to create the final 4 Apo-Si-K170A-InfA constructs.

Apo-Si-K170A-InfA constructs comprise:
1. K170A-MF03-PB1 sense strand sequence
(SEQ ID NO: 16)
5′-phosphate(Apo-Si-K170A) GATACTGAATCTTGGACAAAAGAAA-3′
antisense strand sequence
(SEQ ID NO: 17)
5′-phosphate(Apo-Si-K170A) TTTCTTTTGTCCAAGATTCAGTATCGA-3′
2. K170A-MF13-PB1 sense strand sequence
(SEQ ID NO: 18)
5′-phosphate(Apo-Si-K170A) TGAGAAAGATGATGACTAATTCACA-3′
antisense strand sequence
(SEQ ID NO: 19)
5′-phosphate(Apo-Si-K170A) TGTGAATTAGTCATCATCTTTCTCACA-3′
3. K170A-MF43-PB2 sense strand sequence
(SEQ ID NO: 20)
5′-phosphate(Apo-Si-K170A) GCAATAGGGTTGAGGATTAGCTCAT-3′
antisense strand sequence
(SEQ ID NO: 21)
5′-phosphate(Apo-Si-K170A) ATGAGCTAATCCTCAACCCTATTGCTG-3′
4. K170A-MF45-PB2 sense strand sequence
(SEQ ID NO: 22)
5′-phosphate(Apo-Si-K170A) GGATGATGGCAATGAGATACCCAAT-3′
antisense strand sequence
(SEQ ID NO: 23)
5′-phosphate(Apo-Si-K170A) ATTGGGTATCTCATTGCCATCATCCAC-3′
Additional Apo-Si-K170A-InfA constructs with initial indications of biological
activity.
5. K170A-MF44-PB2 sense strand sequence
(SEQ ID NO: 24)
5′-phosphate(Apo-Si-K170A) GGAACAAGCCGTAGACATATGCAAG-3′
antisense strand sequence
(SEQ ID NO: 25)
5′-phosphate(Apo-Si-K170A) CTTGCATATGTCTACGGCTTGTTCCTC-3′
6. K170A-MF33-PB2 sense strand sequence
(SEQ ID NO: 26)
5′-phosphate(Apo-Si-K170A) GCAGAAGAGTAGACATAAACCCTGG-3′
antisense strand sequence
(SEQ ID NO: 27)
5′-phosphate(Apo-Si-K170A) CCAGGGTTTATGTCTACTCTTCTGCGT-3′
7. K170A-MF31-PB2 sense strand sequence
(SEQ ID NO: 28)
5′-phosphate(Apo-Si-K170A) CACAAGAAGATTGCATGATAAAAGC-3′
antisense strand sequence
(SEQ ID NO: 29)
5′-phosphate(Apo-Si-K170A) GCTTTTATCATGCAATCTTCTTGTGAA-3′
8. K170A-MF01-PB1 sense strand sequence
(SEQ ID NO: 30)
5′-phosphate(Apo-Si-K170A) GAATCAACAAGGAAGAAAATTGAGA-3′
antisense strand sequence
(SEQ ID NO: 31)
5′-phosphate(Apo-Si-K170A) TCTCAATTTTCTTCCTTGTTGATTCAT-3′
K170A-MF02-PB1 sense strand sequence
(SEQ ID NO: 32)
5′-phosphate(Apo-Si-K170A) GCATTGACCTGAAGTATTTCAATGA-3′
antisense strand sequence
(SEQ ID NO: 33)
5′-phosphate(Apo-Si-K170A) TCATTGRAAATACTTCAGGTCAATGCTT-3′
10. K170A-MF03-PB1 sense strand sequence
(SEQ ID NO: 34)
5′-phosphate(Apo-Si-K170A) GATACTGAATCTTGGACAAAAGAAA-3′
antisense strand sequence
(SEQ ID NO: 35)
5′-phosphate(Apo-Si-K170A) TTTCTTTTGTCCAAGATTCAGTATCGA-3′
Virus strain and Cell Line

In this assay, we used Influenza A/California/07/2009 (H1N1) pdm09 (VR-1894) and A/California/2/2014 (H3N2) (VR-1938) strains infecting MDCK cells (CCL-34). All virus strains and cell were obtained from ATCC. A/California/2/2014 (H3N2)(VR-1938) strain PB1 and PB2 genes were compared with those of Influenza A/Perth/16/2009 (H3N2) to ensure that there are no significant mismatches that may hinder the compatibility of the siRNA to the strain.

### Assay Solutions

OptiPRO serum-free medium, supplemented with 2 mM L-glutamine, 1% non-essential amino acids (all from Gibco), and 1% penicillin-streptomycin (HyClone) was used as assay medium.

Constructs were obtained in lyophilized dry powder and resuspended in RNase- and DNase-free Molecular Water to a stock concentration of 0.3 mM prior to study initiation. Dilutions of constructs to working solutions was done in OptiPRO assay medium (as described). Each tested Construct was assayed at 8 concentrations, 2-fold serial dilutions, starting from 30 μM, in triplicates.

### Antiviral Assay

MDCK cells were seeded in 96 well plates, in 100 μL per well of assay medium, at a density of 15,000 cells per well (96 well plate) and cultured at 37° C. and 5% CO₂ for 5 hours. Test articles, prepared according to their respective serial dilutions, were added into the cells, at a final volume of 150 μl per well. The resulting cell cultures were incubated for additional 24 hours.

After 24 h, the supernatants were removed and then cells were infected with influenza at the MOI=0.012 for H1N1 strain and MOI=0.005 for H3N2 strain. Infection was performed in assay medium containing trypsin. For the test articles test wells, assay medium was added. The final volume of the cell culture was 200 μl per well. The final concentration of trypsin was 2.5 μg/mL. The resulting cell cultures were incubated at 35° C., and 5% CO2 for additional 5 days until virus infection in the virus control (cells infected with virus, without compound treatment) displays significant CPE. Finally, at D7, the CPE was measured by CCK8 (Life-iLab) following the manufacturer's manual. The plates were read at 2 and 3 hours post the CCK8 treatment, respectively. The antiviral activity of compounds is calculated based on the protection of the virus-induced CPE at each concentration normalized by the virus control.

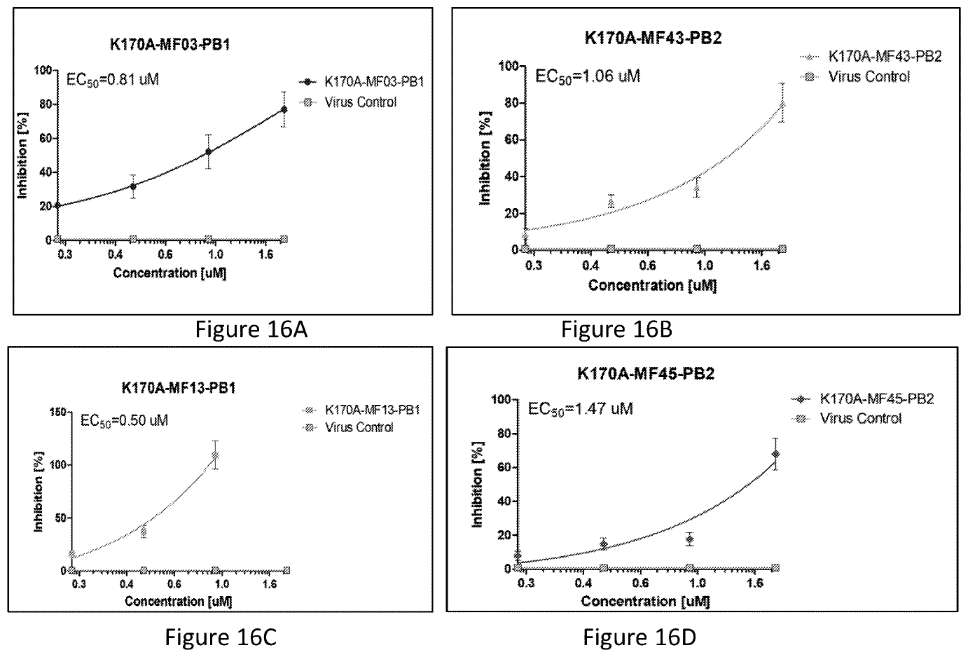

The results of this experiment (presented in FIGS. 16A-D) show that Apo-Si-MNM-dsiRNA constructs showed good dose-response between concentrations 0.3-1.6 M, with 50% effective concentration (EC₅₀ of 1.47 μM to 0.5 μM).

Based on the results of examples 10 and 7, it is presumed that the conjugates of the invention can be implemented for treatment of viral infections, such as respiratory viral infections.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A conjugate, having the structure of general Formula (I): 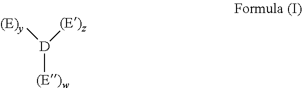
including pharmaceutically acceptable salts, hydrates, solvates and metal chelates thereof, wherein:
D is a polynucleic acid;
each of y, z and w is an integer, independently selected from 0, 1, 2, 3 or 4, wherein at least one of y, z or w is different than 0;
E, E′, or E″ can be the same or different, each having independently a structure of general Formula (II) including pharmaceutically acceptable salts, hydrates, solvates and metal chelates thereof, 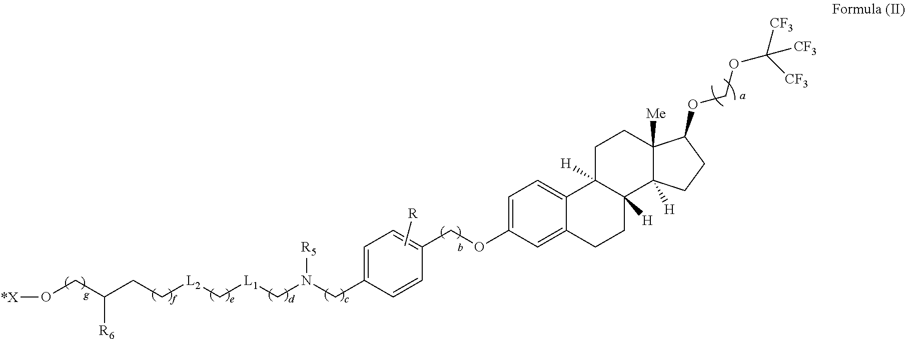
wherein:
X absent or represents 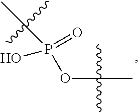 [SMILES: *OP(*)(=O)O]
a salt thereof, or both;
Each of a, b, c, d, e, f, g is an integer independently selected from 1, 2, 3, 4, 5, 6, 7, 8, 9, 10;
R represents one or more substituents, each independently is selected from H, F, Cl, Br, I provided that at least one of the one or more substituents is F;
R5 is H or a straight or branched C1-C5 alkyl;
R6 is selected from H, hydroxy alkyl, and —(CH₂)ₙR′; wherein n is an integer selected from 1, 2, 3, 4, 5, 6, 7, 8, 9, 10, and wherein R′ is selected from a bond, H, and phosphate;
L1 is a linker selected from —NH—C(═O)—, —C(═O)NH—, —C(═O)—S, —S—S— and —S—C(═O);
L2 is a linker selected from —O—, —S—, —CH₂—, or is absent;
* is the attachment point to D;
or wherein E, E′, or E″ can be the same or different, each having independently a structure of general Formula (III) including pharmaceutically acceptable salts, hydrates, solvates and metal chelates thereof, wherein:
Each of h, i, j, k is an integer independently selected from 1, 2, 3, 4, 5, 6, 7, 8, 9, 10;
R7 is selected from H, hydroxy alkyl, and —(CH₂)ₙR′; wherein n is an integer selected from 1, 2, 3, 4, 5, 6, 7, 8, 9, 10, and wherein R′ is selected from a bond, H, and phosphate;
L3 is a linker selected from —NH—C(═O)—, —C(═O)NH—, —S—S— and —NH—C(═O);
L4 is a linker selected from —O—, —S—, —CH₂— or is absent;
* is the attachment point to D;

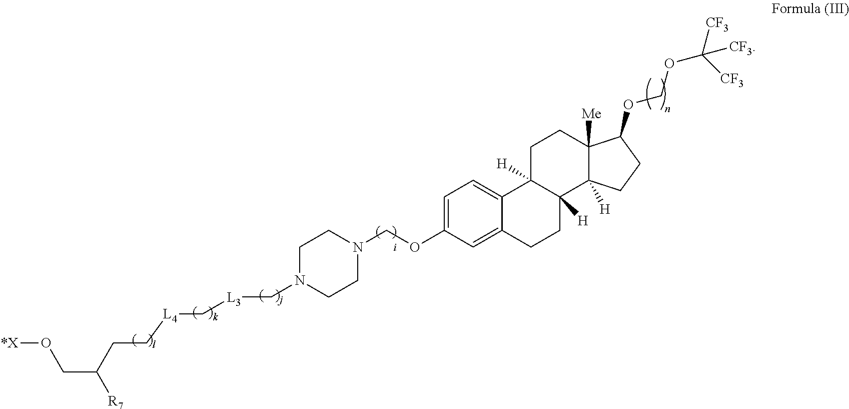

2. The conjugate of claim 1, wherein L1 is S—S and L2 is —O— or —S—.

3. The conjugate of claim 1, wherein E, E′, or E″ can be the same or different, each having independently a structure of general Formula (IIa) including pharmaceutically acceptable salts, hydrates, solvates and metal chelates thereof: 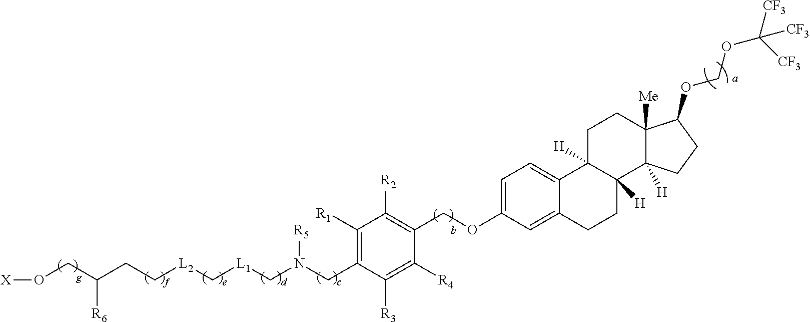
wherein each R1-R4 is independently selected from H, F, Cl, Br, I provided that at least one of R1-R4 is F.

4. The conjugate of claim 3, wherein R1, R2, R4 are H and R3 is F.

5. The conjugate of claim 3, wherein R1, R3, R4 are H and R2 is F and R1 is F.

6.-12. (canceled)

13. The conjugate of claim 1, wherein R5 is Me.

14. The conjugate of claim 1, wherein R6 is —CH₂OH, or 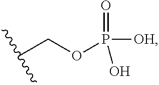 [SMILES: *COP(=O)(O)O]
including any salt thereof.

15. The conjugate of claim 1, wherein R6 is H, or —CH₂OH; and wherein L3 is —S—S— and L4 is —O—; or wherein L3 is selected from —S—C(═O), —C(═O)S—, —NH—C(═O), and —C(═O)NH— and L4 is absent.

16. (canceled)

17. The conjugate of claim 1, wherein L1 is selected from —S—C(═O), —C(═O)S—, —NH—C(═O), and —C(═O)NH—; and wherein L2 is —O— or absent.

18. The conjugate of claim 1, wherein R7 is H, —CH₂OH, or 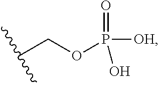 [SMILES: *COP(=O)(O)O]
including any salt thereof.

19. (canceled)

20. (canceled)

21. (canceled)

22. The conjugate of claim 1, wherein a sum of y, z and w is 2; wherein: (i) each of E, E′, or E″ is independently represented by Formula (I) or by Formula (IIa), a is 3 and b is 1; or (ii) each of E, E′, or E″ is independently represented by Formula (III) and h is 3; and wherein D is an oligonucleotide drug.

23. (canceled)

24. (canceled)

25. (canceled)

26. The conjugate of claim 1, wherein E, E′, or E″ is selected from:

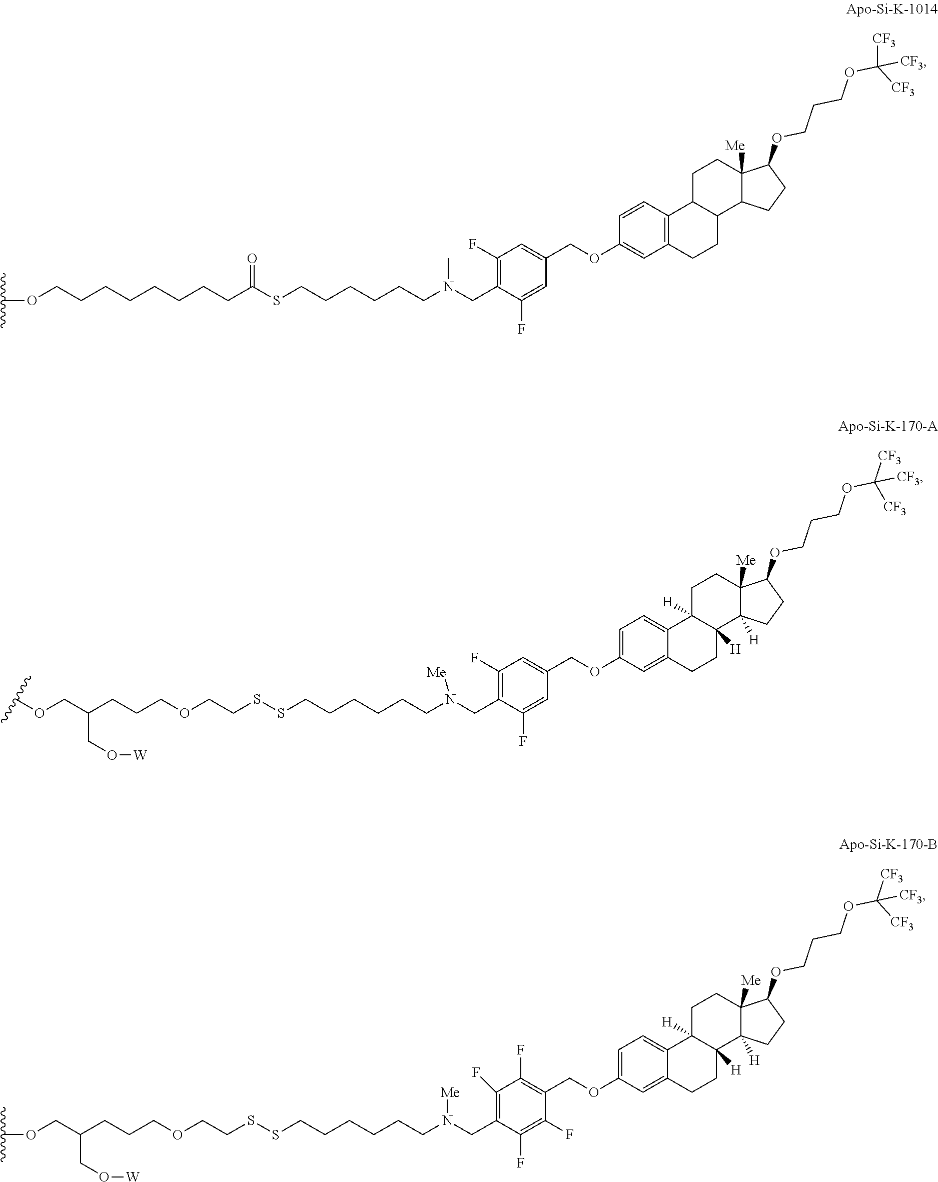

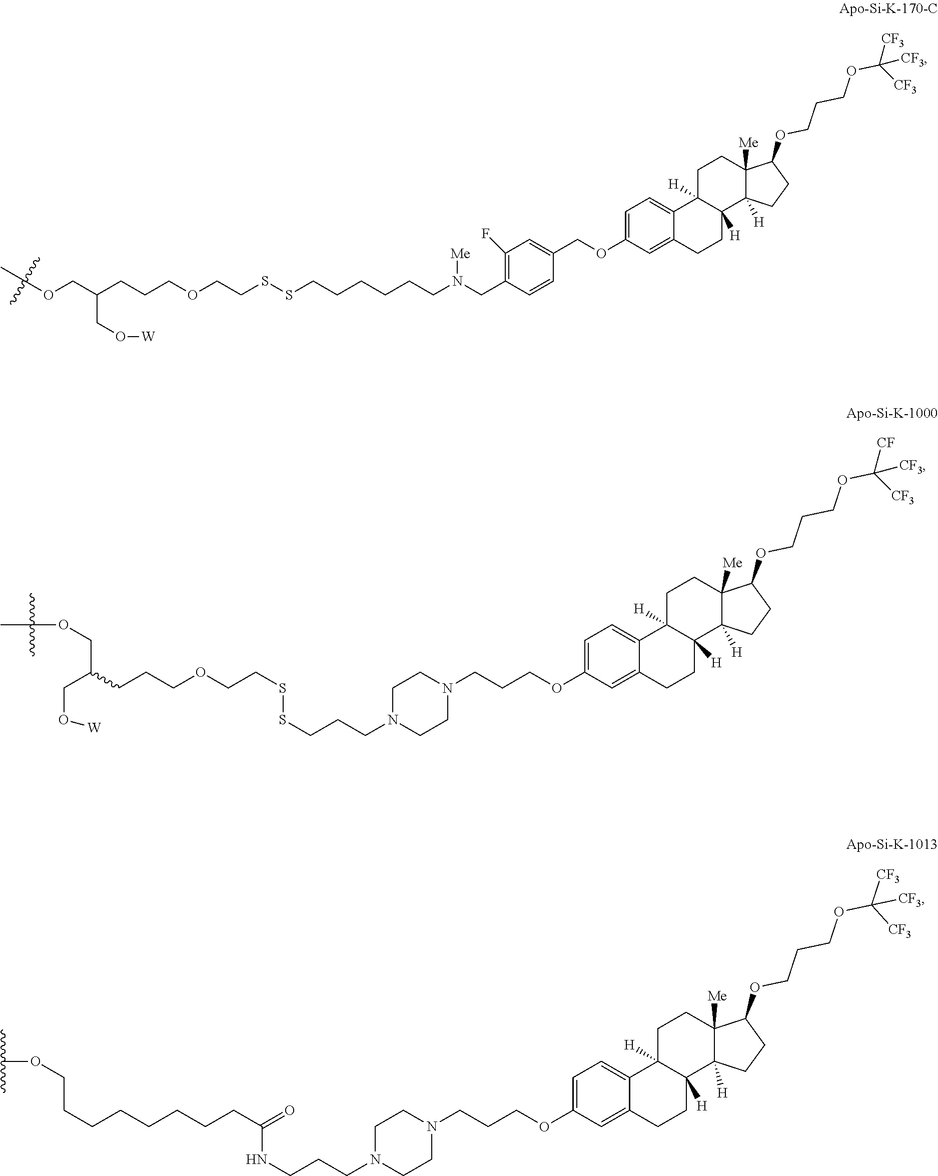

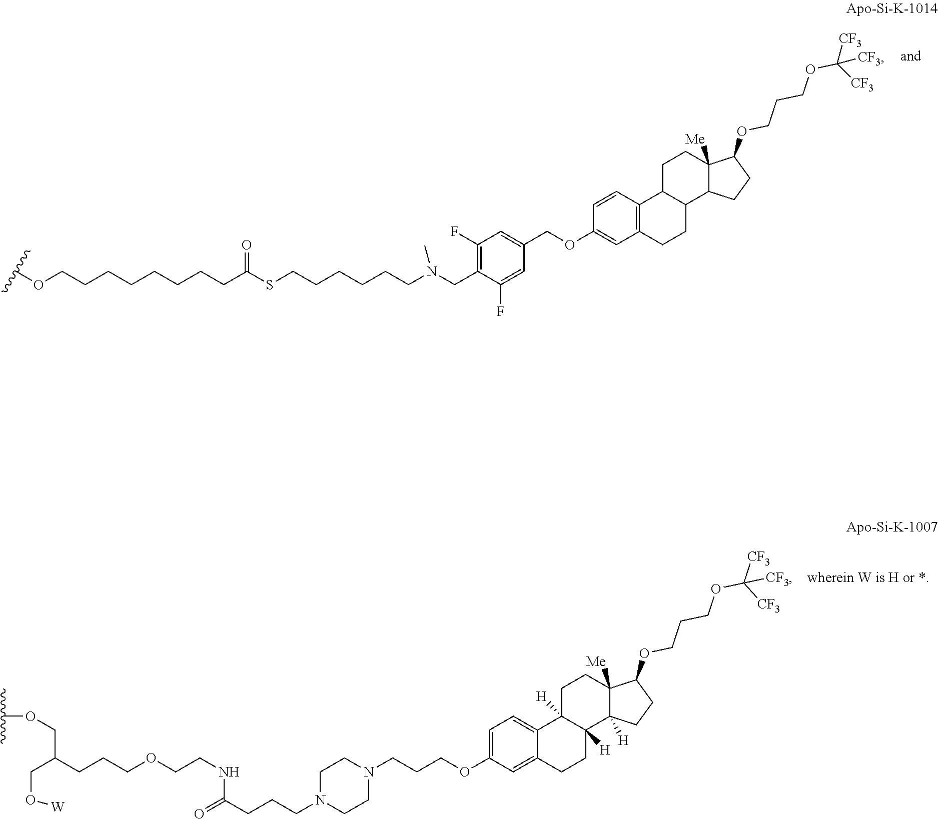

27. A precursor of the conjugate of Formula II of claim 1, wherein
said precursor is represented by Formula (IV): 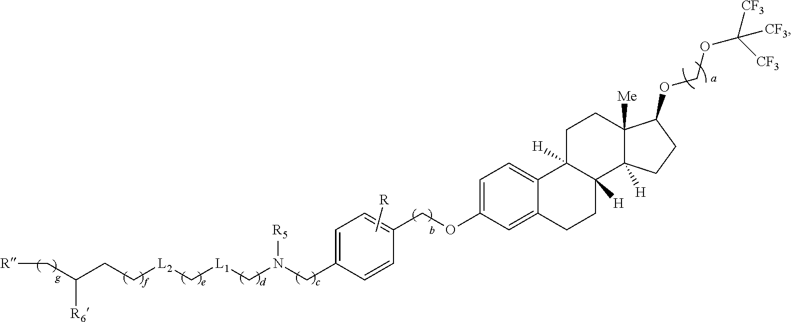
wherein R″ is a phosphoramidite, and wherein each of R6′ and R7′ is independently H, —OX′, or —(CH₂)ₙOX′, wherein n is an integer between 1 and 10, and X′ is H or a hydroxy protecting group.

28. A precursor of the conjugate of Formula III of claim 1, wherein said precursor is represented by Formula (V): 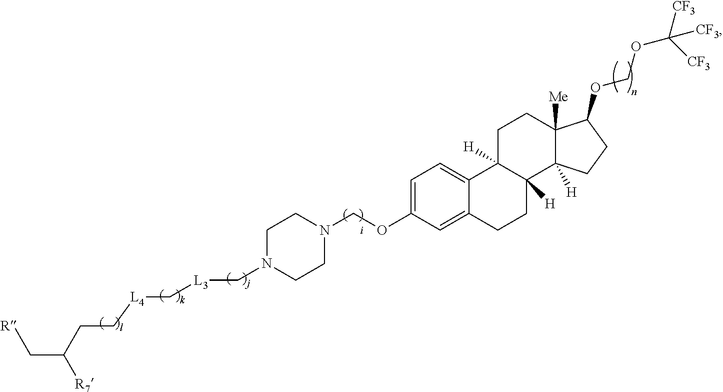
wherein R″ is a phosphoramidite, and wherein each of R6′ and R7′ is independently H, —OX′, or —(CH₂)ₙOX′, wherein n is an integer between 1 and 10, and X′ is H or a hydroxy protecting group.

29. A pharmaceutical composition, comprising one or more conjugates of claim 1 and a pharmaceutically acceptable carrier.

30. The pharmaceutical composition of claim 29, comprising a therapeutically effective amount of the one or more conjugates.

31. (canceled)

32. (canceled)

33. (canceled)

34. (canceled)

35. (canceled)

36. (canceled)

37. A method for delivering a polynucleic acid into a cell of a subject comprising administering to said subject the pharmaceutical composition of claim 29.

38. A method for treating or preventing a disease within a subject, the method comprising administering to said subject a therapeutically effective amount of the pharmaceutical composition of claim 29, thereby treating or preventing said disease.

39. (canceled)

40. The method of claim 38, wherein said disease comprises a genetic disease, a viral disease, cancer, a CNS disease, an inflammatory disease, a lung disease, or any combination thereof.


SEQUENCE LISTING (35 sequences)

SEQ ID NO: 1 - RNA, 25 nt, Synthetic construct
  source 1..25: mol_type = other RNA
  modified_base 1: mod_base = OTHER; note = 2'-O-methyladenosine
  modified_base 4: mod_base = cm
  modified_base 6: mod_base = gm
  modified_base 12: mod_base = cm
  modified_base 14: mod_base = um
  modified_base 16: mod_base = um
  modified_base 18: mod_base = cm
  modified_base 24: mod_base = cm
  modified_base 2: mod_base = cm
accctgaagttcatctgcaccaccg

SEQ ID NO: 2 - RNA, 27 nt, Synthetic construct
  source 1..27: mol_type = other RNA
  modified_base 3: mod_base = gm
  modified_base 9: mod_base = cm
  modified_base 11: mod_base = gm
  modified_base 13: mod_base = um
  modified_base 23: mod_base = gm
  modified_base 25: mod_base = um
  modified_base 26: mod_base = cm
  modified_base 27: mod_base = OTHER; note = 2'-O-methyladenosine
cggtggtgcagatgaacttcagggtca

SEQ ID NO: 3 - RNA, 25 nt, Synthetic construct
  source 1..25: mol_type = other RNA
  modified_base 1: mod_base = um
  modified_base 4: mod_base = gm
  modified_base 6: mod_base = OTHER; note = 2'-O-methyladenosine
  modified_base 12: mod_base = OTHER; note = 2'-O-methyladenosine
  modified_base 14: mod_base = cm
  modified_base 16: mod_base = OTHER; note = 2'-O-methyladenosine
  modified_base 18: mod_base = um
  modified_base 24: mod_base = um
  modified_base 2: mod_base = gm
tgtgcaaccagaacaaatcagactg

SEQ ID NO: 4 - RNA, 27 nt, Synthetic construct
  source 1..27: mol_type = other RNA
  modified_base 3: mod_base = gm
  modified_base 9: mod_base = um
  modified_base 11: mod_base = um
  modified_base 13: mod_base = um
  modified_base 23: mod_base = OTHER; note = 2'-O-methyladenosine
  modified_base 25: mod_base = OTHER; note = 2'-O-methyladenosine
  modified_base 26: mod_base = gm
  modified_base 27: mod_base = um
cagtctgatttgttctggttgcacagt

SEQ ID NO: 5 - RNA, 27 nt, Synthetic construct
  source 1..27: mol_type = other RNA
  modified_base 3: mod_base = OTHER; note = 2'-O-methyladenosine
  modified_base 6: mod_base = cm
  modified_base 8: mod_base = gm
  modified_base 14: mod_base = cm
  modified_base 16: mod_base = um
  modified_base 18: mod_base = um
  modified_base 20: mod_base = cm
  modified_base 26: mod_base = cm
  modified_base 4: mod_base = cm
ttaccctgaagttcatctgcaccaccg

SEQ ID NO: 6 - RNA, 25 nt, Synthetic construct
  source 1..25: mol_type = other RNA
  modified_base 1: mod_base = gm
  modified_base 4: mod_base = OTHER; note = 2'-O-methyladenosine
  modified_base 6: mod_base = gm
  modified_base 12: mod_base = OTHER; note = 2'-O-methyladenosine
  modified_base 14: mod_base = OTHER; note = 2'-O-methyladenosine
  modified_base 16: mod_base = OTHER; note = 2'-O-methyladenosine
  modified_base 18: mod_base = um
  modified_base 24: mod_base = um
  modified_base 2: mod_base = OTHER; note = 2'-O-methyladenosine
gaaatggtgctatagatttaccatt

SEQ ID NO: 7 - RNA, 27 nt, Synthetic construct
  source 1..27: mol_type = other RNA
  modified_base 3: mod_base = um
  modified_base 9: mod_base = OTHER; note = 2'-O-methyladenosine
  modified_base 11: mod_base = cm
  modified_base 13: mod_base = OTHER; note = 2'-O-methyladenosine
  modified_base 23: mod_base = um
  modified_base 25: mod_base = cm
  modified_base 26: mod_base = OTHER; note = 2'-O-methyladenosine
  modified_base 27: mod_base = cm
aatggtaaatctatagcaccatttcac

SEQ ID NO: 8 - RNA, 25 nt, Synthetic construct
  source 1..25: mol_type = other RNA
  modified_base 1: mod_base = gm
  modified_base 4: mod_base = um
  modified_base 6: mod_base = um
  modified_base 12: mod_base = OTHER; note = 2'-O-methyladenosine
  modified_base 14: mod_base = gm
  modified_base 16: mod_base = cm
  modified_base 18: mod_base = OTHER; note = 2'-O-methyladenosine
  modified_base 24: mod_base = OTHER; note = 2'-O-methyladenosine
  modified_base 2: mod_base = gm
ggctcttagcaaagtcaagttgaat

SEQ ID NO: 9 - RNA, 27 nt, Synthetic construct
  source 1..27: mol_type = other RNA
  modified_base 3: mod_base = um
  modified_base 9: mod_base = um
  modified_base 11: mod_base = OTHER; note = 2'-O-methyladenosine
  modified_base 13: mod_base = um
  modified_base 23: mod_base = gm
  modified_base 25: mod_base = cm
  modified_base 26: mod_base = OTHER; note = 2'-O-methyladenosine
  modified_base 27: mod_base = um
attcaacttgactttgctaagagccat

SEQ ID NO: 10 - RNA, 25 nt, Synthetic construct
  source 1..25: mol_type = other RNA
  modified_base 1: mod_base = cm
  modified_base 4: mod_base = OTHER; note = 2'-O-methyladenosine
  modified_base 6: mod_base = gm
  modified_base 12: mod_base = cm
  modified_base 14: mod_base = cm
  modified_base 16: mod_base = OTHER; note = 2'-O-methyladenosine
  modified_base 18: mod_base = um
  modified_base 24: mod_base = um
  modified_base 2: mod_base = um
ctaaaggacctcacgaattttgctc

SEQ ID NO: 11 - RNA, 27 nt, Synthetic construct
  source 1..27: mol_type = other RNA
  modified_base 3: mod_base = gm
  modified_base 9: mod_base = um
  modified_base 11: mod_base = cm
  modified_base 13: mod_base = um
  modified_base 23: mod_base = um
  modified_base 25: mod_base = gm
  modified_base 26: mod_base = um
  modified_base 27: mod_base = OTHER; note = 2'-O-methyladenosine
gagcaaaattcgtgaggtcctttagta

SEQ ID NO: 12 - RNA, 25 nt, Synthetic construct
  source 1..25: mol_type = other RNA
  modified_base 1: mod_base = OTHER; note = 2'-O-methyladenosine
  modified_base 4: mod_base = um
  modified_base 6: mod_base = cm
  modified_base 12: mod_base = gm
  modified_base 14: mod_base = um
  modified_base 16: mod_base = cm
  modified_base 18: mod_base = OTHER; note = 2'-O-methyladenosine
  modified_base 24: mod_base = cm
  modified_base 2: mod_base = gm
agatgctttctgttacaacatggcc

SEQ ID NO: 13 - RNA, 26 nt, Synthetic construct
  source 1..26: mol_type = other RNA
  modified_base 3: mod_base = cm
  modified_base 9: mod_base = OTHER; note = 2'-O-methyladenosine
  modified_base 11: mod_base = um
  modified_base 13: mod_base = OTHER; note = 2'-O-methyladenosine
  modified_base 22: mod_base = um
  modified_base 24: mod_base = um
  modified_base 25: mod_base = gm
  modified_base 26: mod_base = OTHER; note = 2'-O-methyladenosine
ggccatgttgtaacagaaagctctga

SEQ ID NO: 14 - RNA, 25 nt, Synthetic construct
  source 1..25: mol_type = other RNA
  modified_base 1: mod_base = cm
  modified_base 4: mod_base = cm
  modified_base 6: mod_base = um
  modified_base 12: mod_base = cm
  modified_base 14: mod_base = um
  modified_base 16: mod_base = um
  modified_base 18: mod_base = cm
  modified_base 24: mod_base = OTHER; note = 2'-O-methyladenosine
  modified_base 2: mod_base = cm
ccacttgaaaccttctactaatcaa

SEQ ID NO: 15 - RNA, 27 nt, Synthetic construct
  source 1..27: mol_type = other RNA
  modified_base 3: mod_base = gm
  modified_base 9: mod_base = um
  modified_base 11: mod_base = OTHER; note = 2'-O-methyladenosine
  modified_base 13: mod_base = OTHER; note = 2'-O-methyladenosine
  modified_base 23: mod_base = um
  modified_base 25: mod_base = gm
  modified_base 26: mod_base = cm
  modified_base 27: mod_base = OTHER; note = 2'-O-methyladenosine
ttgattagtagaaggtttcaagtggca

SEQ ID NO: 16 - RNA, 25 nt, Synthetic construct
  source 1..25: mol_type = other RNA
  modified_base 1: mod_base = gm
  modified_base 4: mod_base = OTHER; note = 2'-O-methyladenosine
  modified_base 6: mod_base = um
  modified_base 12: mod_base = um
  modified_base 14: mod_base = gm
  modified_base 16: mod_base = OTHER; note = 2'-O-methyladenosine
  modified_base 18: mod_base = OTHER; note = 2'-O-methyladenosine
  modified_base 24: mod_base = OTHER; note = 2'-O-methyladenosine
  modified_base 2: mod_base = OTHER; note = 2'-O-methyladenosine
gatactgaatcttggacaaaagaaa

SEQ ID NO: 17 - RNA, 27 nt, Synthetic construct
  source 1..27: mol_type = other RNA
  modified_base 3: mod_base = um
  modified_base 9: mod_base = gm
  modified_base 11: mod_base = cm
  modified_base 13: mod_base = OTHER; note = 2'-O-methyladenosine
  modified_base 23: mod_base = OTHER; note = 2'-O-methyladenosine
  modified_base 25: mod_base = cm
  modified_base 26: mod_base = gm
  modified_base 27: mod_base = OTHER; note = 2'-O-methyladenosine
tttcttttgtccaagattcagtatcga

SEQ ID NO: 18 - RNA, 25 nt, Synthetic construct
  source 1..25: mol_type = other RNA
  modified_base 2: mod_base = gm
  modified_base 4: mod_base = gm
  modified_base 6: mod_base = OTHER; note = 2'-O-methyladenosine
  modified_base 12: mod_base = OTHER; note = 2'-O-methyladenosine
  modified_base 14: mod_base = gm
  modified_base 16: mod_base = cm
  modified_base 18: mod_base = OTHER; note = 2'-O-methyladenosine
  modified_base 24: mod_base = cm
tgagaaagatgatgactaattcaca

SEQ ID NO: 19 - RNA, 27 nt, Synthetic construct
  source 1..27: mol_type = other RNA
  modified_base 3: mod_base = um
  modified_base 9: mod_base = OTHER; note = 2'-O-methyladenosine
  modified_base 11: mod_base = um
  modified_base 13: mod_base = OTHER; note = 2'-O-methyladenosine
  modified_base 23: mod_base = um
  modified_base 25: mod_base = OTHER; note = 2'-O-methyladenosine
  modified_base 26: mod_base = cm
  modified_base 27: mod_base = OTHER; note = 2'-O-methyladenosine
tgtgaattagtcatcatctttctcaca

SEQ ID NO: 20 - RNA, 25 nt, Synthetic construct
  source 1..25: mol_type = other RNA
  modified_base 1: mod_base = gm
  modified_base 4: mod_base = OTHER; note = 2'-O-methyladenosine
  modified_base 6: mod_base = OTHER; note = 2'-O-methyladenosine
  modified_base 12: mod_base = gm
  modified_base 14: mod_base = gm
  modified_base 16: mod_base = OTHER; note = 2'-O-methyladenosine
  modified_base 18: mod_base = um
  modified_base 24: mod_base = OTHER; note = 2'-O-methyladenosine
  modified_base 2: mod_base = cm
gcaatagggttgaggattagctcat

SEQ ID NO: 21 - RNA, 27 nt, Synthetic construct
  source 1..27: mol_type = other RNA
  modified_base 3: mod_base = gm
  modified_base 9: mod_base = OTHER; note = 2'-O-methyladenosine
  modified_base 11: mod_base = cm
  modified_base 13: mod_base = um
  modified_base 23: mod_base = um
  modified_base 25: mod_base = cm
  modified_base 26: mod_base = um
  modified_base 27: mod_base = gm
atgagctaatcctcaaccctattgctg

SEQ ID NO: 22 - RNA, 25 nt, Synthetic construct
  source 1..25: mol_type = other RNA
  modified_base 1: mod_base = gm
  modified_base 4: mod_base = um
  modified_base 6: mod_base = OTHER; note = 2'-O-methyladenosine
  modified_base 12: mod_base = OTHER; note = 2'-O-methyladenosine
  modified_base 14: mod_base = gm
  modified_base 16: mod_base = gm
  modified_base 18: mod_base = um
  modified_base 24: mod_base = OTHER; note = 2'-O-methyladenosine
  modified_base 2: mod_base = gm
ggatgatggcaatgagatacccaat

SEQ ID NO: 23 - RNA, 27 nt, Synthetic construct
  source 1..27: mol_type = other RNA
  modified_base 3: mod_base = um
  modified_base 9: mod_base = um
  modified_base 11: mod_base = um
  modified_base 13: mod_base = OTHER; note = 2'-O-methyladenosine
  modified_base 23: mod_base = um
  modified_base 25: mod_base = cm
  modified_base 26: mod_base = OTHER; note = 2'-O-methyladenosine
  modified_base 27: mod_base = cm
attgggtatctcattgccatcatccac

SEQ ID NO: 24 - RNA, 25 nt, Synthetic construct
  source 1..25: mol_type = other RNA
  modified_base 1: mod_base = gm
  modified_base 4: mod_base = OTHER; note = 2'-O-methyladenosine
  modified_base 6: mod_base = OTHER; note = 2'-O-methyladenosine
  modified_base 12: mod_base = um
  modified_base 14: mod_base = gm
  modified_base 16: mod_base = cm
  modified_base 18: mod_base = um
  modified_base 24: mod_base = OTHER; note = 2'-O-methyladenosine
  modified_base 2: mod_base = gm
ggaacaagccgtagacatatgcaag

SEQ ID NO: 25 - RNA, 27 nt, Synthetic construct
  source 1..27: mol_type = other RNA
  modified_base 3: mod_base = um
  modified_base 9: mod_base = um
  modified_base 11: mod_base = um
  modified_base 13: mod_base = um
  modified_base 23: mod_base = um
  modified_base 25: mod_base = cm
  modified_base 26: mod_base = um
  modified_base 27: mod_base = cm
cttgcatatgtctacggcttgttcctc

SEQ ID NO: 26 - RNA, 25 nt, Synthetic construct
  source 1..25: mol_type = other RNA
  modified_base 1: mod_base = gm
  modified_base 4: mod_base = gm
  modified_base 6: mod_base = OTHER; note = 2'-O-methyladenosine
  modified_base 12: mod_base = gm
  modified_base 14: mod_base = cm
  modified_base 16: mod_base = um
  modified_base 18: mod_base = OTHER; note = 2'-O-methyladenosine
  modified_base 24: mod_base = gm
  modified_base 2: mod_base = cm
gcagaagagtagacataaaccctgg

SEQ ID NO: 27 - RNA, 27 nt, Synthetic construct
  source 1..27: mol_type = other RNA
  modified_base 3: mod_base = OTHER; note = 2'-O-methyladenosine
  modified_base 9: mod_base = um
  modified_base 11: mod_base = um
  modified_base 13: mod_base = um
  modified_base 23: mod_base = um
  modified_base 25: mod_base = cm
  modified_base 26: mod_base = gm
  modified_base 27: mod_base = um
ccagggtttatgtctactcttctgcgt

SEQ ID NO: 28 - RNA, 25 nt, Synthetic construct
  source 1..25: mol_type = other RNA
  modified_base 1: mod_base = cm
  modified_base 4: mod_base = OTHER; note = 2'-O-methyladenosine
  modified_base 6: mod_base = gm
  modified_base 12: mod_base = um
  modified_base 14: mod_base = cm
  modified_base 16: mod_base = um
  modified_base 18: mod_base = OTHER; note = 2'-O-methyladenosine
  modified_base 24: mod_base = gm
  modified_base 2: mod_base = OTHER; note = 2'-O-methyladenosine
cacaagaagattgcatgataaaagc

SEQ ID NO: 29 - RNA, 27 nt, Synthetic construct
  source 1..27: mol_type = other RNA
  modified_base 3: mod_base = um
  modified_base 9: mod_base = cm
  modified_base 11: mod_base = um
  modified_base 13: mod_base = cm
  modified_base 23: mod_base = gm
  modified_base 25: mod_base = gm
  modified_base 26: mod_base = OTHER; note = 2'-O-methyladenosine
  modified_base 27: mod_base = OTHER; note = 2'-O-methyladenosine
gcttttatcatgcaatcttcttgtgaa

SEQ ID NO: 30 - RNA, 25 nt, Synthetic construct
  source 1..25: mol_type = other RNA
  modified_base 1: mod_base = gm
  modified_base 4: mod_base = um
  modified_base 6: mod_base = OTHER; note = 2'-O-methyladenosine
  modified_base 12: mod_base = gm
  modified_base 14: mod_base = OTHER; note = 2'-O-methyladenosine
  modified_base 16: mod_base = OTHER; note = 2'-O-methyladenosine
  modified_base 18: mod_base = OTHER; note = 2'-O-methyladenosine
  modified_base 24: mod_base = gm
  modified_base 2: mod_base = OTHER; note = 2'-O-methyladenosine
gaatcaacaaggaagaaaattgaga

SEQ ID NO: 31 - RNA, 27 nt, Synthetic construct
  source 1..27: mol_type = other RNA
  modified_base 3: mod_base = um
  modified_base 9: mod_base = um
  modified_base 11: mod_base = cm
  modified_base 13: mod_base = um
  modified_base 23: mod_base = um
  modified_base 25: mod_base = cm
  modified_base 26: mod_base = OTHER; note = 2'-O-methyladenosine
  modified_base 27: mod_base = um
tctcaattttcttccttgttgattcat

SEQ ID NO: 32 - RNA, 25 nt, Synthetic construct
  source 1..25: mol_type = other RNA
  modified_base 1: mod_base = gm
  modified_base 4: mod_base = um
  modified_base 6: mod_base = gm
  modified_base 12: mod_base = OTHER; note = 2'-O-methyladenosine
  modified_base 14: mod_base = gm
  modified_base 16: mod_base = OTHER; note = 2'-O-methyladenosine
  modified_base 18: mod_base = um
  modified_base 24: mod_base = gm
  modified_base 2: mod_base = cm
gcattgacctgaagtatttcaatga

SEQ ID NO: 33 - RNA, 28 nt, Synthetic construct
  source 1..28: mol_type = other RNA
  modified_base 3: mod_base = OTHER; note = 2'-O-methyladenosine
  modified_base 9: mod_base = OTHER; note = 2'-O-methyladenosine
  modified_base 11: mod_base = um
  modified_base 13: mod_base = cm
  modified_base 23: mod_base = OTHER; note = 2'-O-methyladenosine
  modified_base 25: mod_base = gm
  modified_base 26: mod_base = cm
  modified_base 27: mod_base = um
tcattgraaatacttcaggtcaatgctt

SEQ ID NO: 34 - RNA, 25 nt, Synthetic construct
  source 1..25: mol_type = other RNA
  modified_base 1: mod_base = gm
  modified_base 4: mod_base = OTHER; note = 2'-O-methyladenosine
  modified_base 6: mod_base = um
  modified_base 12: mod_base = um
  modified_base 14: mod_base = gm
  modified_base 16: mod_base = OTHER; note = 2'-O-methyladenosine
  modified_base 18: mod_base = OTHER; note = 2'-O-methyladenosine
  modified_base 24: mod_base = OTHER; note = 2'-O-methyladenosine
  modified_base 2: mod_base = OTHER; note = 2'-O-methyladenosine
gatactgaatcttggacaaaagaaa

SEQ ID NO: 35 - RNA, 27 nt, Synthetic construct
  source 1..27: mol_type = other RNA
  modified_base 3: mod_base = um
  modified_base 9: mod_base = gm
  modified_base 11: mod_base = cm
  modified_base 13: mod_base = OTHER; note = 2'-O-methyladenosine
  modified_base 23: mod_base = OTHER; note = 2'-O-methyladenosine
  modified_base 25: mod_base = cm
  modified_base 26: mod_base = gm
  modified_base 27: mod_base = OTHER; note = 2'-O-methyladenosine
tttcttttgtccaagattcagtatcga